(19) United States
(12) Patent Application Publication
(10) Pub. No.: US 2011/0110958 A1
(43) Pub. Date: May 12, 2011

(54) COMPOSITIONS AND METHODS FOR THE TREATMENT OF NEOPLASIA

(75) Inventors: Jingxin Qiu, Buffalo, NY (US); Mingli Yang, Gainesville, FL (US); David A. Ostrov, Gainesville, FL (US); W. Stratford May, Gainesville, FL (US)

(73) Assignee: UNIVERSITY OF FLORIDA RESEARCH FOUNDATION, Gainesville, FL (US)

(21) Appl. No.: 12/677,705

(22) PCT Filed: Sep. 11, 2008
(86) PCT No.: PCT/US08/10729

§ 371 Date: Jan. 27, 2011

Related U.S. Application Data
(60) Provisional application No. 60/993,243, filed on Sep. 11, 2007.
(60) Provisional application No. 61/003,390, filed on Nov. 16, 2007.

(51) Int. Cl. A61K 39/395; A61K 31/17; A61K 31/4188; A61K 31/713; A61K 31/7088; C12N 5/09; C12Q 1/02; C12Q 1/18; G01N 33/68; A61K 48/00; A61P 35/00; A61P 35/02; G06G 7/58

(52) U.S. Cl. 424/174.1; 514/596; 514/598; 514/393; 514/44 A; 435/375; 435/29; 435/32; 435/7.1; 514/44 R; 703/11

(57) ABSTRACT

The invention provides compositions and methods for the treatment of neoplasias that are cytotoxic to neoplastic cells or that modulate JAZ expression, subcellular localization, or biological activity.

### CROSS-REFERENCE TO RELATED APPLICATION

This application claims the benefit of the following U.S. Provisional Application Nos. 60/993,243, filed Sep. 11, 2007, and 61/003,390, filed Nov. 16, 2007, the entire contents of each of which are incorporated herein by reference.

### BACKGROUND OF THE INVENTION

Gliomas are the most common primary central nervous system (CNS) tumors in adults, representing 63% of all primary CNS tumors. These genetically and clinically heterogeneous tumors are difficult to treat because of their infiltrative growth and resistance to currently available therapies. In fact, only twenty-six percent of patients diagnosed as having a glioblastoma multiforme that are treated with standard therapies, which include radiotherapy and temozolomide, remain alive two years following their diagnosis. For decades, little improvement in methods of treatment for glioma has been made. This is likely due to a limited understanding of the biology underlying the disease. Novel therapeutic strategies are urgently required for glioma treatment.

### SUMMARY OF THE INVENTION

As described below, the present invention features compositions and methods for the treatment of neoplasias (e.g., glioma, glioblastoma, hepatocellular carcinoma, lung cancer, leukemia, colon cancer, and prostate carcinoma).

In one aspect, the invention features a pharmaceutical composition for the treatment of a neoplasia containing an effective amount of a substituted diphenyl urea compound (e.g., J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl) urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea) and a pharmaceutically acceptable excipient. In one embodiment, the diphenyl urea compound is represented by the formula:

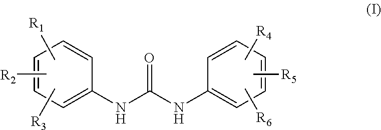

in which

R₁, R₂, R₃, R₄, R₅, and R₆ are independently H, Cl, or OH;

or a pharmaceutically acceptable salt or solvate thereof. In one embodiment, at least one of R₁, R₂, or R₃ is Cl or OH. In another embodiment, at least one of R₄, R₅, or R₆ is Cl or OH. In yet another embodiment, the diphenyl urea compound is represented by the formula:

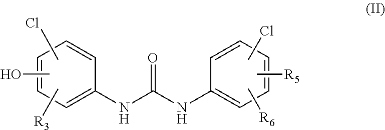

in which R₃, R₄, R₅, and R₆ are independently one or more of H, Cl, and OH. In yet another embodiment, the diphenyl urea compound is represented by the formula:

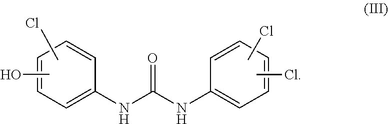

In still another embodiment, the diphenyl urea compound is represented by the formula:

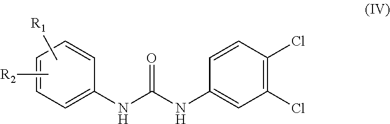

in which

R₁ and R₂ are independently one or more of H, Cl, and OH;

or a pharmaceutically acceptable salt or solvate thereof. In yet another embodiment, R₁ is H or Cl and R₂ is OH. In yet another embodiment, the diphenyl urea compound is represented by the formula:

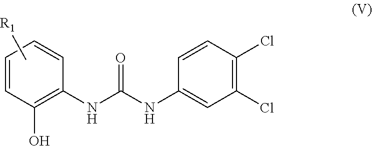

in which R₁ is H or Cl.

In various embodiments of the previous aspects, the compound is J1, J1-b, J1-h, a compound of Table 3, or any other compound delineated herein.

In another aspect, the invention features a method of ameliorating a neoplasia (e.g., glioma, glioblastoma, hepatocellular carcinoma, lung cancer, leukemia, and prostate carcinoma) in a subject, the method involving administering to the subject an effective amount of a compound of any of the formulae herein.

In yet another aspect, the invention features a method of ameliorating a neoplasia in a subject, the method involving administering to the subject an effective amount of an agent that reduces the expression or biological activity of a JAZ polypeptide. In one embodiment, the agent specifically binds a JAZ polypeptide. In another embodiment, the method further involves the step of administering temozolomide to the subject. In still another embodiment, the agent is a JAZ inhibitory nucleic acid molecule that is an antisense, siRNA or shRNA molecule. In another embodiment, the agent is an antibody.

In yet another aspect, the invention features a method of ameliorating a neoplasia in a subject, the method involving administering to the subject an effective amount of an agent that binds to a structural binding pocket sequence of a JAZ polypeptide. In one embodiment, the agent is a compound delineated herein (e.g., J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea). In another embodiment, the agent is an antibody.

In yet another aspect, the invention features a method of reducing the survival of a neoplastic cell, the method involving contacting the neoplastic cell with a compound delineated herein.

In embodiments of any of the previous aspects, the method further involves administering to the subject an effective amount of temozolomide. In one embodiment, the compound reduces neoplastic cell proliferation or invasiveness. In another embodiment, the neoplasia is a glioma, glioblastoma multiforme, lung cancer or colon cancer.

In yet another aspect, the invention provides a method for identifying an agent that binds to a JAZ polypeptide or fragment thereof, the method involving performing a computational fitting operation between the agent and a three-dimensional representation of a binding site of the JAZ polypeptide; and quantifying an association between the chemical entity and the three-dimensional representation of the structural binding pocket, thereby identifying an agent that reduces the biological activity of the JAZ polypeptide. In one embodiment, the JAZ polypeptide comprises a JAZ structural binding pocket. In another embodiment, the agent reduces JAZ biological activity, or disrupts subcellular localization.

In yet another aspect, the invention features a method for identifying an agent that reduces JAZ biological activity, the method involving generating a three-dimensional representation of a structural binding pocket sequence using the atomic coordinates of JAZ at least a portion of the JAZ polypeptide; and employing the three-dimensional structure to design or select a JAZ antagonist, thereby identifying an agent that reduces JAZ biological activity. In one embodiment, the agent binds a JAZ structural binding pocket sequence. In another embodiment, the agent is a substituted diphenyl urea compound, such as a J1, J1-b, J1-h, a compound of Table 3, or any other substituted diphenyl urea compound. In yet another embodiment, the method further involves contacting the agent with a JAZ expressing cell and detecting a reduction in JAZ expression or biological activity. In yet another embodiment, the cell is a cell in vitro or in vivo.

In yet another aspect, the invention features a method for identifying an agent for the treatment of a neoplasia, the method involving contacting a JAZ expressing cell with a candidate agent; and detecting a reduction in the level of biological activity of a JAZ polypeptide in the contacted cell relative to a control cell, thereby identifying the agent as treating a neoplasia. In one embodiment, the JAZ biological activity is detected by assaying proliferation, survival or invasiveness of a neoplastic cell. In another embodiment, the agent specifically binds the JAZ polypeptide. In yet another embodiment, the agent specifically binds the JAZ structural binding pocket sequence. In yet another embodiment, the agent is an antibody or a substituted diphenyl urea compound.

In yet another aspect, the invention features a method for identifying an agent for the treatment of a neoplasia, the method involving contacting a JAZ expressing cell with the agent; and detecting a reduction in JAZ expression in the cell relative to an untreated control cell, thereby identifying the agent as treating a neoplasia. In one embodiment, the reduction is in JAZ transcription or translation. In another embodiment, the agent is a JAZ inhibitory nucleic acid molecule (e.g., an antisense, siRNA, or shRNA molecule).

In yet another aspect, the invention features a kit for the treatment of a neoplasia, the kit containing an effective amount of a compound delineated herein or shown in Table 3 and directions for using the compound for the treatment of a neoplasia. In yet another embodiment, the kit further includes temozolomide.

In another aspect, the invention features a pharmaceutical composition containing the pharmaceutical composition of a previous aspect, and an effective amount of an inhibitory nucleic acid molecule that is any one or more of an antisense, siRNA or shRNA molecule, wherein said inhibitory nucleic acid molecule is sufficiently complementary to the transcript of a gene selected from the group consisting of PBEF1, HSPA5, ANXA1, TPI1, ANXA5, HSPA9, TGM2, NPM1, RPLP2, HSPA1B, VIM, LMNA and HIST2H3PS2 to reduce the expression of said gene in a mammalian cell, and a pharmaceutically-acceptable carrier.

In yet another aspect, the invention provides a computer for producing a three-dimensional representation of

a) a molecule or molecular complex, wherein said molecule or molecular complex comprises a binding site defined by structure coordinates of amino acid residues of the JAZ protein; or

b) a three-dimensional representation of a homologue of said molecule or molecular complex, wherein said homologue comprises a binding pocket that has a root mean square deviation from the backbone atoms of said amino acids of not more than about 2.0 angstroms, wherein said computer comprises:

(i) a machine-readable data storage medium comprising a data storage material encoded with machine-readable data, wherein said data comprises the structure coordinates of structure coordinates of amino acid residues of the JAZ protein;

(ii) a working memory for storing instructions for processing said machine-readable data;

(iii) a central-processing unit coupled to said working memory and to said machine-readable data storage medium for processing said machine readable data into said three-dimensional representation; and

(iv) a display coupled to said central-processing unit for displaying said three-dimensional representation. In one embodiment, the structure coordinates comprise those shown in FIG. 26.

In yet another aspect, the invention features a method for evaluating the potential of a chemical entity to associate with a) a molecule or molecular complex comprising a binding pocket defined by structure coordinates of amino acid residues of the JAZ protein, or b) a homologue of said molecule or molecular complex, wherein said homologue comprises a binding pocket that has a root mean square deviation from the backbone atoms of said amino acids of not more than 2.0 angstroms,

the method comprising the steps of:

i) employing computational means to perform a fitting operation between the chemical entity and a binding pocket of the molecule or molecular complex; and

ii) analyzing the results of the fitting operation to quantify the association between the chemical entity and the binding pocket.

In yet another aspect, the invention provides a method for evaluating the potential of a chemical entity to bind with a) a molecule or molecular complex comprising a binding pocket defined by structure coordinates of one or more of amino acid residues of the JAZ protein, or b) a homologue of said molecule or molecular complex, wherein said homologue comprises a binding pocket that has a root mean square deviation from the backbone atoms of said amino acids of not more than 2.0 angstroms,

the method comprising the steps of:

i) employing computational means to perform a fitting operation between the chemical entity and a binding pocket of the molecule or molecular complex; and

ii) analyzing the results of the fitting operation to quantify the association between the chemical entity and the binding pocket.

In various embodiments of the above methods, the JAZ structural binding pocket contains at least a fragment of the following amino acid sequence:

vsg aqpvgreeve hmiqknqclf tntqckvcca llisesqk. In other embodiments, a JAZ structural binding pocket comprises one or more of GLN 17, HIS14, GLU10, GLU13, Arg9, GLU38, LYS41, ARG25, and ILe161.

The invention provides compositions and methods for the treatment of neoplasias, such as glioma, glioblastoma, hepatocellular carcinoma, lung cancer, leukemia, colon cancer, and prostate carcinoma, that are cytotoxic to neoplastic cells or that modulate JAZ expression, subcellular localization, or biological activity.

In yet another aspect, the invention provides a method of ameliorating a neoplasia in a subject, the method involving administering to the subject an effective amount of the pharmaceutical composition of a previous aspect and an effective amount of an agent that reduces the expression or biological activity of a polypeptide that is any one or more of PBEF1, HSPA5, ANXA1, TPI1, ANXA5, HSPA9, TGM2, NPM1, RPLP2, HSPA1B, VIM, LMNA and HIST2H3PS2.

In yet another aspect, the invention provides a method of ameliorating a neoplasia in a subject (e.g., a human patient), the method comprising administering to the subject an effective amount of an agent that increases the expression or biological activity of an ATF-4 or CHOP polypeptide. In one embodiment, the agent is a mammalian expression vector encoding human ATF-4 or CHOP.

In yet another aspect, the invention provides a method for identifying a compound for the treatment of a neoplasia, the method involving contacting a JAZ expressing cell with a candidate agent; detecting a reduction in JAZ expression in the cell relative to an untreated control cell, and detecting a decrease in the expression PLOD2 or any other marker delineated herein, thereby identifying the agent as treating a neoplasia.

In yet another aspect, the invention provides a pharmaceutical composition comprising the pharmaceutical composition of any previous aspect, and an effective amount of an inhibitory nucleic acid molecule that is any one or more of an antisense, siRNA or shRNA molecule, where the inhibitory nucleic acid molecule is sufficiently complementary to the transcript of a gene selected from the group consisting of PBEF1, HSPA5, ANXA1, TPI1, ANXA5, HSPA9, TGM2, NPM1, RPLP2, HSPA1B, VIM, LMNA and HIST2H3PS2 to reduce the expression of said gene in a mammalian cell, and a pharmaceutically-acceptable carrier.

In various embodiments of any of the above aspects, the agent specifically binds the JAZ polypeptide (e.g., a JAZ structural binding pocket). In still other embodiments, the method further involves administering temozolomide to the subject. In still other embodiments, the agent is an inhibitory nucleic acid molecule selected from the group consisting of an antisense, siRNA or shRNA molecule.

In particular embodiments of any of the above aspects, compounds of the invention are substituted diphenyl urea compounds, such as J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea, or metabolites or analogs of such compounds.

In various embodiments of any of the above aspects, the invention provides methods for ameliorating a neoplasia in a subject, the method comprising administering to the subject an effective amount of a compound capable of disrupting binding of a JAZ protein with a nucleic acid molecule, such that a neoplasia (e.g., glioma, glioblastoma, hepatocellular carcinoma, lung cancer, leukemia, melanoma, colon cancer, and prostate carcinoma) is ameliorated in the subject. In still other embodiments, Other features and advantages of the invention will be apparent from the detailed description, and from the claims.

### DEFINITIONS

By “JAZ polypeptide” is meant a zinc finger protein or a functional fragment thereof having at least about 85% identity to NCBI Accession No. AAD52018 and having JAZ biological activity. JAZ is described, for example, in Yang et al., J Biol Chem 1999:274: 27399-27406, which is incorporated herein in its entirety. In other embodiments, a JAZ has substantial identity to NCBI Accession No. AAD52018. In other embodiments, a fragment of JAZ comprises a JAZ structural binding pocket sequence.

By “JAZ structural binding pocket” is meant a fragment of the JAZ polypeptide that mediates agent binding. The JAZ structural binding pocket described herein was based on an atomic homology model used for selection of the compounds. As described herein, selection of the compounds was defined by a structural pocket based on the following sequence of a human JAZ structural binding pocket: vsg aqpvgreeve hmiqknqclf tntqckvcca llisesqk. This peptide comprises the following residues, which may mediate compound binding: GLN 17, HIS14, GLU10, GLU13, Arg9, GLU38, LYS41, ARG25, and ILe16. These residues are numbered relative to the aforementioned peptide sequence.

In other embodiments, the JAZ structural binding pocket sequence includes at least about 5, 10, 15, 20, 25, 30, 40, or 50 amino acids flanking the aforementioned sequence at the amino and carboxy termini of the JAZ structural binding pocket sequence sequence.

An exemplary human JAZ polypeptide amino acid sequence follows:

| | |
|---|---|
| 1 | meypapatvq aadggaagpy sssellegqe pdgvrfdrer |
| | arrlweavsg aqpvgreeve |
| 61 | hmiqknqclf tntqckvcca llisesqkla hyqskkhank |
| | vkrylaihgm etlkgetkkl |
| 121 | dsdqkssrsk dknqccpicn mtfsspvvaq shylgkthak |
| | nlklkqqstk vealhqnrem |
| 181 | idpdkfcslc hatfndpvma qqhyvgkkhr kqetklklma |
| | rygrladpav tdfpagkgyp |
| 241 | cktckivlns ieqvqahvsg fkhknqspkt vasslgqipm |
| | qrqpiqkdst tled |

By “ATF-4 polypeptide” is meant a polypeptide having at least about 85% amino acid identity to NCBI Accession No. NP_{—}001666 and having transcriptional regulatory activity. An exemplary sequence of a human ATF-4 polypeptide follows:

| | |
|---|---|
| 1 | mtemsflsse vlvgdlmspf dqsglgaees lgllddylev |
| | akhfkphgfs sdkakagsse |
| 61 | wlavdglvsp snnskedafs gtdwmlekmd lkefdldall |
| | giddletmpd dllttlddtc |
| 121 | dlfaplvqet nkqppqtvnp ighlpesltk pdqvapftfl |
| | qplplspgvl sstpdhsfsl |
| 181 | elgsevdite gdrkpdytay vamipqcike edtpsdndsg |
| | icmspesylg spqhspstrg |
| 241 | spnrslpspg vlcgsarpkp ydppgekmva akvkgekldk |
| | klkkmeqnkt aatryrqkkr |
| 301 | aeqealtgec kelekkneal keradslake iqylkdliee |
| | vrkargkkrv p |

By “ATF-4 nucleic acid molecule” is meant a polynucleotide encoding an ATF-4 polypeptide.

By “CHOP polypeptide” is meant a polypeptide having at least about 85% amino acid identity to NCBI Accession No. NP_{—}004074 or a fragment thereof having transcriptional regulatory activity. An exemplary sequence of a human CHOP polypeptide follows:

| | |
|---|---|
| 1 | maaeslpfsf gtlsswelea wyedlqevls sdenggtyvs |
| | ppgneeeesk ifttldpasl |
| 61 | awlteeepep aevtstsqsp hspdssqssl aqeeeeedqg |
| | rtrkrkqsgh sparagkqrm |
| 121 | kekeqenerk vaqlaeener lkqeierltr eveatrrali |
| | drmvnlhqa |

By “CHOP nucleic acid molecule” is meant a polynucleotide encoding an CHOP polypeptide.

By “substantially identical” is meant a polypeptide or nucleic acid molecule exhibiting at least 85% identity to a reference amino acid sequence (for example, any one of the amino acid sequences described herein) or nucleic acid sequence (for example, any one of the nucleic acid sequences described herein). Preferably, such a sequence is at least 85%, 90%, 95%, 99% or even 100% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e.sup.-3 and e.sup.-100 indicating a closely related sequence.

By “JAZ biological activity” is meant preferentially binding to a nucleic acid molecule, dsRNA or RNA/DNA hybrids or any other activity that involves the growth, proliferation, invasiveness, or aggressiveness of a neoplasia.

By “JAZ nucleic acid molecule” is meant any nucleic acid molecule that encodes a JAZ polypeptide. The sequence of an exemplary JAZ nucleic acid molecule is described at NCBI Accession No. NP_{—}036411.

“Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilo n, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kDa) and one “heavy” chain (up to about 70 kDa). Antibodies exist, for example, as intact immunoglobulins or as a number of well-characterized fragments produced by digestion with various peptidases. While various antibody fragments are defined in terms of the digestion of an intact antibody, one of skill in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al., J. Mol. Biol., 296:57-86, 2000; McCafferty et al., Nature, 348:2-4, 1990), for example. For preparation of antibodies—recombinant, monoclonal, or polyclonal antibodies—any technique known in the art can be used with this invention (see, for example, Kohler & Milstein, Nature, 256(5517):495-497, 1975; Kozbor et al., Immunology Today, 4:72, 1983; Cole et al., pp. 77-96 in Monoclonal Antibodies and Cancer Therapy, Alan R. Liss, Inc., 1998).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology also can be used to identify antibodies and heteromeric Fab fragments that specifically bind to selected antigens (see, for example, McCafferty et al., Nature, 348:2-4, 1990; Marks et al., Biotechnology, 10(7):779-783, 1992). The term antibody is used in the broadest sense including agonist, antagonist, and blocking or neutralizing antibodies.

By “binding to” a molecule is meant having a physicochemical affinity for that molecule. Binding may be measured by any of the methods of the invention, e.g., using an in vitro translation binding assay.

By “compound” is meant any small molecule chemical compound, antibody, nucleic acid molecule, or polypeptide, or fragments thereof.

Illustrative neoplasms for which the invention can be used to detect new therapeutic compounds (or confirm activity of existing compounds) include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In one embodiment, screening methods of the invention identify compositions that are useful for treating central nervous system, glial, colon, breast or lung cancer.

By “computer modeling” is meant the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model.

By a “computer system” is meant the hardware means, software means and data storage means used to analyse atomic coordinate data. The minimum hardware means of the computer-based systems of the present invention comprises a central processing unit (CPU), input means, output means and data storage means. Desirably a monitor is provided to visualise structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media” is meant any media which can be read and accessed directly by a computer e.g. so that the media is suitable for use in the above-mentioned computer system. The media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

By “fitting”, is meant determining by automatic, or semi-automatic means, interactions between one or more atoms of an agent molecule and one or more atoms or binding sites of JAZ, and determining the extent to which such interactions are stable. Various computer-based methods for fitting are described further herein.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “marker” is meant any protein or polynucleotide having an alteration in expression level or activity that is associated with a disease or disorder. In particular embodiments, markers of the invention include those showing an alteration detected with a microarray, including but not limited to ATF-4, CHOP, PBEF1, HSPA5, ANXA1, TPI1, ANXA5, HSPA9, TGM2, NPM1, RPLP2, HSPA1B, VIM, LMNA and HIST2H3PS2.

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides. A microarray may include, for example, nucleic acid molecules representing all, or a subset, of the open reading frames of an organism, or of the polypeptides that those open reading frames encode. In one embodiment, the nucleic acid molecules of the array are defined as having a common region of the genome having limited homology to other regions of an organism's genome.

By “reduces” or “increases” is meant a negative or positive alteration, respectively, of at least 10%, 25%, 50%, 75%, or 100% relative to a reference.

By “root mean square deviation” is meant the square root of the arithmetic mean of the squares of the deviations from the mean.

By “reducing cell survival” is meant to inhibit the viability of a cell or to induce cell death relative to a reference cell.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “subject” is meant a mammal, including, but not limited to, a human or non-human mammal, such as a bovine, equine, canine, ovine, or feline.

By “specifically binds” is meant a compound or antibody that recognizes and binds a polypeptide of the invention, but which does not substantially recognize and bind other molecules in a sample, for example, a biological sample, which naturally includes a polypeptide of the invention.

As used herein, the terms “treat,” treating,” “treatment,” and the like refer to reducing or ameliorating a disorder and/or symptoms associated therewith. By “ameliorate” is meant decrease, suppress, attenuate, diminish, arrest, or stabilize the development or progression of a disease. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of a compound described herein may range from about 1 μg/Kg to about 5000 mg/Kg body weight. Effective doses will also vary depending on route of administration, as well as the possibility of co-usage with other agents.

### BRIEF DESCRIPTION OF THE DRAWINGS

FIGS. 1A and 1B show JAZ expression. FIG. 1A is a Northern blot showing that JAZ is ubiquitously expressed in a variety of tissues, including brain. FIG. 1B includes two panels of micrographs of NIH-3T3 cells and MCF-1 cells. The panel on the lefts shows immunofluorescence with JAZ antibody. The panel on the right shows DAPI fluorescence in nuclei and nucleoli. The colocalization of the JAZ staining with DAPI fluorescence indicates that JAZ is a nuclear/nucleolar protein in vitro.

FIGS. 2A-2C show immunofluorescence and immunohistochemistry studies of JAZ subcellular localization in human central nervous system (CNS) tissues. FIG. 2A shows that JAZ is predominantly localized to the cytoplasm in human CNS neurons (arrowhead) and in glioma cells. Normal astrocytes and oligodendrocytes (arrows) do not express JAZ. FIG. 2B shows representative images of JAZ immunostaining on the tissue microarray (TMA). FIG. 2C is a graph that quantitates image analysis results of JAZ immunostaining on the TMA. JAZ has a tumor grade-dependent expression pattern, with glioblastoma (GBM, WHO grade IV) having the highest expression. :p<0.05 compared with non-neoplastic control cortex (Cortex), non-neoplastic control white matter (White Matter), and anaplastic astrocytoma (AA, WHO Grade III) groups. +:p<0.05 compared with White Matter group.

FIGS. 3A and 3B are illustrations of proposed mechanisms associated with cell death and glioma development. FIG. 3A is a schematic diagram that proposes a model in which JAZ contributes to cell cycle arrest and/or cell death through a p53-dependent mechanism. FIG. 3B is a model depicting one proposed mechanism by which hypoxia/ischemia may lead to the formation of pseudopalisading necrosis and contribute to glioma invasiveness/aggressiveness.

FIGS. 4A-4D are fourteen micrographs showing JAZ subcellular localization in human CNS tissues using immunofluorescence. FIGS. 4A and 4B show JAZ immunofluorescence, DAPI fluorescence, and merged images. This analysis shows that JAZ localized to the cytoplasm expression in human CNS neurons (FIG. 4A) and glioma (FIG. 4B). FIG. 4C shows JAZ and Bcl-xL immunofluorescence, DAPI fluorescence, and merged images showing that JAZ localized to the mitochondria in CNS neurons (FIG. 4C). FIG. 4D shows p53 and JAZ immunofluorescence, DAPI fluorescence, and merged images. JAZ and p53 were co-expressed within the glioma cells (FIG. 4D) but were present in different sub-cellular compartments.

FIGS. 5A-5D are micrographs showing JAZ Immunohistochemistry. FIG. 5A shows that JAZ nuclear staining (arrows) is increased in pseudopalisading tumor cells. FIG. 5B shows double immunofluorescence of JAZ and p53, which was cytoplasmic or nuclear (white arrow), respectively. JAZ staining did not co-localize with nuclear p53 expression in glioma tumor cells. FIG. 5C shows an immunofluorescence analysis of JAZ in U87MG and T98G glioma cells. Interestingly, these cells showed strong nuclear JAZ expression in addition to cytoplasmic JAZ signals. Immunofluorescence analysis of p53 showed no p53 expression in U87MG cells, but strong nuclear p53 expression in T98G cells. FIG. 5D shows an immunofluorescence analysis of JAZ in U118MG and GL261 glioma cells. These cells showed nuclear JAZ expression in addition to cytoplasmic JAZ.

FIGS. 6A and 6B show the DNA methylation status of the JAZ promoter in control brain tissue (gray matter and white matter), normal human astrocytes (Lonza Bioscience), and human glioma cell lines by bisulfite genomic sequencing. The JAZ CpG island is largely unmethylated in all the specimens examined. The meaning of each symbol in this figure is denoted in the following legend: ◯: unmethylated CpG. :methylated CpG. Numbering is relative to the transcription start site as defined using the NCBI Map View. FIG. 6B shows the results of methylation specific PCR. This analysis includes the methylation status of the JAZ promoter in normal gray and white matter, normal human astrocytes (NHA), T98G glioma cell line, and glioblastoma GBM samples. The JAZ promoter is unmethylated in all specimens examined. M: methylated. U: unmethylated. 7: methylated control; 8: un-methylated control.

FIGS. 7A-7F are micrographs showing hematoxylin and eosin (H&E) (FIGS. 7A and 7B) and immunohistochemistry analysis of JAZ (FIGS. 7D and 7E) in normal cerebral cortex (FIGS. 7A and 7D) and hypoxia/ischemia damaged cerebral cortex (FIGS. 7B and 7E). Increased nuclear JAZ expression was observed in hypoxic neurons (arrow) compared with predominantly cytoplasmic JAZ expression in normal cortical neurons (arrow head). FIGS. 7C and 7C shows H&E and immunohistochemistry analysis of JAZ. FIG. 7F shows staining in a cultured brain slice, where increased nuclear JAZ expression was observed in hypoxia/ischemia damaged neurons (arrow) compared with predominantly cytoplasmic JAZ in adjacent normal cortical neurons (insert, arrow head).

FIGS. 8A-8D show the effects of inhibition of JAZ by JAZ siRNA in glioma cells. FIG. 8A includes sixteen micrographs showing results of double immunofluorescence staining in WT4 glioma cells, which had both cytoplasmic and nuclear expression. WT4 glioma cells expressed wild type p53 without the presence of doxycycline. Adding doxycycline (2 μg/ml) into the culture medium completely shut down the p53 expression. JAZ siRNA transfection led to decreased JAZ expression in WT4 tumor cells at 48 hours after transfection. FIG. 8B is a graph showing the results of a cell viability assays. Non-p53-expressing WT4 cells exhibited more rapid growth when compared with p53-expressing WT4 cells. FIG. 8C is a graph showing that there was a reduction in the number of viable tumor cells that are p53-expressing and p53-non-expressing WT4 cells after transfection with JAZ siRNA (40 pmoles), although the decreases were not statistically significant. FIG. 8D is a graph showing the results of a cell viability assay. A significant decrease in cell growth was observed for both U87MG and T98G glioma cell lines after JAZ siRNA (40 pmoles) transfection. Forty-eight hours after the transfection, there was a 46.4% decrease in viability of U87MG glioma cells and a 39.5% decrease in viability of T98G glioma cells when compared with control siRNA transfected groups (p<0.05).

FIG. 9 illustrates a strategy for a molecular docking and drug design model.

FIGS. 10A-10C provide molecular models showing the structure of JAZ's first two zing finger domains (FIGS. 10A and 10B) and the way J1 docks to the JAZ polypeptide (FIG. 10C).

FIGS. 11A-11G show the effects of inhibition of JAZ by a structure-based JAZ inhibitor “J1” on glioma cells. FIG. 11A shows the 2D chemical structure and chemical name of J1, N′-(2-hydroxy-phenyl)-N-(3,4-dichlorophenyl)Urea. FIG. 11B is a graph showing the results of cell viability assays, which showed that there were time-dependent significant reduction of viable tumor cells in both p53-expressing and p53-non-expressing WT4 cells after JAZ inhibitor-J1 treatment (25 μM). FIG. 11C is a graph showing that there was a reduction of viable U87 cells at 48 hours after J1 treatment (10 μM). Twenty-four hours after JAZ siRNA transfection, U87 cells were treated with J1 at 10 mM for additional 24 hours. This J1 treatment did not lead to further reduction of viable U87 cells suggesting that J1's action was dependent on JAZ expression. FIG. 11D is a graph showing the results of cell viability assays, which showed that there was a time- and dose-dependent significant reduction of viable tumor cells in U87MG cells after J1 treatment. FIG. 11E is a graph showing the results of cell viability assays, which showed that there was time- and dose-dependent significant reduction of viable tumor cells in mutant-type p53-expressing T98G cells after J1 treatment. FIG. 11F shows that there was increased DNA laddering formation in U87MG glioma cells following J1 treatment (25 μM). FIG. 11G shows Western blot results. The Western blot showed that J1 treatment (25 μM) did not alter JAZ, p53, and Bax protein levels.

FIG. 12 provides a comparison of GI₅₀ (i.e., the concentration of drug that causes 50% growth inhibition) of J1 with the GI₅₀ of some current FDA approved chemotherapy drugs. J1's efficacy (GI₅₀ at 10 uM) is within the range of many currently FDA-approved chemotherapy drugs. Temozolomide (TMZ), for example, has a GI₅₀ of 100 uM. Thus, J1 is much more potent than TMZ.

FIG. 13 is a table showing that J1 is cytotoxic to lung cancer, colon cancer, and leukemia cells.

FIGS. 14A and 14B are tables showing quantitative proteomic analysis of proteome changes 48 hours after J1 (65 μM, NCI) treatment on U87 (FIG. 14A) and U118 (FIG. 14B) glioma cells. To further explore the molecular mechanism of J1's cytotoxic effects on glioma cells, a quantitative proteomics analysis (iTRAQ 4plex) was conducted after J1 treatment. There were four groups: untreated U87 and U118 glioma cells and J1 treated U87 and U118 glioma cells at 48 hours. Each group was in triplicate. Proteomic results showed that HSPA5 (also named as GRP78, Bip) protein levels were consistently and significantly increased in both U87 and U118 glioma cell lines after J1 treatment. Other protein changes are provided in the table. HSPA5 activation is a marker for Unfolded Protein Response (UPR), a pathway that can contribute to cell death or cell survival.

FIG. 15 shows Western blot results of ATF-4 protein levels in U87 glioma cells after J1 treatment. A time- and dose dependent increase in ATF-4 protein levels was observed at 24, 48, and 72 hours after J1 (10 and 25 μM) treatment. ATF-4 is one of the key transcription factors involved in the unfolded protein response pathway, and it is also regarded as a marker for UPR pathway.

FIG. 16 is a schematic diagram showing a model for the potential role of JAZ in the unfolded protein response in tumor cell death.

FIGS. 17A and 17B show immunofluorescence results of ATF-4 and CHOP changes in U87 cells after J1 treatment. FIG. 17A shows that nuclear ATF-4 staining was significantly increased in U87 cells at 12 hours after J1 (25 μM) treatment. FIG. 17B shows that CHOP staining was significantly increased in U87 cells at 24 hours after J1 (25 μM) treatment.

FIG. 18 shows the chemical formulas of J1-b, 1-(4-chloro-2-hydroxy-phenyl)-N-(3,4-dichlorophenyl) Urea, and J1-h, 3-(4-chloro-phenyl)-1-(3,4-dichlorophenyl) Urea, two analogues of J1. The graphs below each compound show the in vitro glioma cytotoxic effects of these analogues. J1-b is one of the major metabolic products of J1-h in human body.

FIG. 19 shows the presence of J1 metabolites in human, monkey, and rat urine, plasma, and bile.

FIG. 20 shows the results of an NCI study of cell line screening.

FIG. 21 show the cytotoxic effects of various J1 analogues on glioma cell lines (U87, U118, and A172).

FIGS. 22A and 22B are graphs showing J1 and J1-h's effects on hepatocytes and hepatocellular carcinoma cells (HepG2). J1 and J1-h exhibit minimal cytotoxic effects on primary cultured hepatocytes at 24 hours after drug treatment (FIG. 22A), while both drugs have significant cytotoxic effects on HepG2 cells (FIGS. 22A and 22B). The cytotoxic effects on HepG2 cells appears to be dose and time-dependent.

FIG. 23 shows J1 and J1-h's effects on DU145 and LNCAP prostate carcinoma cells. J1 and J1-h exhibit strong dose and time-dependent cytotoxic effects on both DU145 and LNCAP cells.

FIG. 24 presents a flow chart showing the role of PBEF in controlling NAD⁺ levels. PBEF was identified to be down-regulated in glioma cells treated with J1.

FIG. 25 presents a 3D histogram that shows the dose- and time-dependent cytotoxic effects of J1, J1-b and TCC administration on GL261 glioma cells.

FIG. 26 provides the atomic coordinates of the atomic homology model of the JAZ structural binding pocket.

### DETAILED DESCRIPTION OF THE INVENTION

The invention features compositions and methods that are useful for the treatment of neoplasias (e.g., glioma, glioblastoma, hepatocellular carcinoma, lung cancer, leukemia, colon cancer, and prostate carcinoma). The invention is based, at least in part, on the discovery that JAZ polypeptide expression is increased and JAZ subcellular localization is altered in glioma cells, and that contacting such cells with a substituted diphenyl urea compound is useful for the treatment of neoplasia. JAZ is a novel nuclear/nucleolar zinc finger protein that can respond to serum deprivation and promote cell death through a p53-dependent mechanism.

To explore JAZ expression and function in the human brain, immunohistochemistry and immunofluorescence analyses were performed using a tissue microarray. Although JAZ was not present, or was present at virtually undetectable levels in normal glia, JAZ was abundantly expressed in high grade gliomas with an unusual, predominantly cytoplasmic distribution. Interestingly, in vivo and in vitro studies showed that neurons undergoing hypoxic damage have strong nuclear labeling for JAZ. Nuclear JAZ expression was observed in pseudopalisading tumor cells of glioblastoma adjacent to hypoxic areas of necrosis, but did not co-localize with increased nuclear p53. Non-hypoxic neurons display only cytoplasmic reactivity. Both cytoplasmic and nuclear JAZ expression was observed in U87MG, T98G, and Tet-off controlled p53 expression WT4 glioma cell lines regardless of p53 status. DNA sequence analysis of JAZ in glioma cells did not reveal any mutation in the JAZ gene. DNA methylation studies did not reveal aberrant DNA methylation in glioma cell lines or human glioma tissues. Unexpectedly, inhibition of JAZ gene expression by JAZ siRNA led to a reduction in glioma tumor cells. The results reported herein indicate that JAZ expression in high grade gliomas under hypoxic conditions likely contributes to glioma cell survival through a p53-independent pathway. In addition, as reported in more detail below, an increase in JAZ expression was observed in tumors of increasing stage, and compounds identified in silico as binding to a JAZ structural binding pocket were toxic to glioma cells in culture. Accordingly, the invention provides compositions and methods for the treatment of subjects identified as having a neoplasia associated with increased JAZ expression or altered JAZ subcellular localization, or subjects identified as having a glioblastoma or other CNS tumor. Compounds that inhibit JAZ expression or biological activity may be used alone for the treatment of neoplasia (e.g., glioma, glioblastoma), or optionally in combination with other conventional treatments for glioblastoma. In one approach, a cytotoxic compound of the invention, such as J1, is administered in combination with TMZ.

### JAZ

JAZ (Just Another Zinc Finger Protein) is a nuclear/nucleolar zinc finger protein. It regulates tumor suppressor gene p53's activity and mediates cell growth arrest and/or apoptosis. Previous studies showed that JAZ is abundantly expressed in the mouse brain (Yang et al., Blood 108:4136-4145, 2006). As reported herein, JAZ was not expressed in normal glial cells, but was abundantly expressed in malignant gliomas with an unusual, predominantly cytoplasmic distribution pattern. JAZ expression levels in gliomas are tumor grade-dependent, with the highest expression in glioblastoma multiforme (WHO grade IV). Interestingly, JAZ subcellular localization was redistributed from the cytoplasm into the nucleus around areas of pseudopalisading necrosis, in which tumor cells are believed to be hypoxic.

Such cytoplasmic to nuclear redistribution was also observed in human CNS neurons under conditions of hypoxia-ischemia. JAZ signals were almost exclusively present in the cell nucleus in the glioma cell lines (U87 and A172). These findings suggest a potential association among stress (hypoxia/ischemia), increased nuclear JAZ signals, and tumor aggressiveness. Recent studies have suggested that hypoxia/ischemia plays an important role in glioma tumorigenesis, including promoting angiogenesis and tumor invasion in glioblastoma multiforme (GBM, WHO grade IV). The hypoxic cellular response is regulated by cell signaling pathways including p53, EGFR, PDGF, and PTEN among others. P53 is a tumor suppressor gene that is frequently mutated in malignant gliomas and alterations in its functions in maintaining genomic stability through DNA repair and mediating cell cycle arrest are believed to be involved in glioma tumorigenesis. The JAZ gene encodes an evolutionarily conserved nuclear/nucleolar zinc finger protein (ZNF346)(Yang et al., J Biol Chem 1999; 274:27399-406), which can preferentially bind to dsRNA (Yang et al., J Biol Chem 1999; 274:27399-406) and may act as a cargo protein in the exportin-5 nuclear transport system (Chen et al., Mol Cell Biol 2004; 24:6608-19). Stress signals like serum starvation induce JAZ expression (Yang et al., Blood 108: 4136-4145, 2006). Recently, JAZ was shown to stimulate p53 transcriptional activity and regulate G1 cell-cycle arrest followed by apoptosis in a p53-dependent mechanism in NFS/N1.H7 murine myeloid cells, M1 murine myeloid leukemic cells, MEF (Mouse Embryonic Fibroblast), and NIH3T3 (Mouse embryonic fibroblast) cells (Yang et al., Blood 108: 4136-4145, 2006). Bax, one of the pro-apoptotic members of the bcl-2 family proteins, was the downstream effector that is responsible for the JAZ-p53 induced cell death (Yang et al., Blood 108: 4136-4145, 2006).

Since JAZ can function through a p53 signaling pathway in response to serum deprivation and is abundantly expressed in rodent brain tissue (Yang et al., J Biol Chem 1999; 274:27399-406)), JAZ expression in glial neoplasms and in the human brain was investigated. As reported herein, JAZ expression is significantly increased in high grade gliomas with a novel, predominantly cytoplasmic (rather than nuclear) distribution. Nuclear JAZ expression was observed in response to hypoxia in both tumor cells and CNS neurons. Neither increased cytoplasmic nor nuclear JAZ expression correlated with increased p53 expression in high grade gliomas. Inhibition of JAZ expression significantly reduced glioma cell growth regardless of the p53 expression status. The results described below indicate that increased JAZ expression in response to hypoxic/ischemic stress in high grade glioma likely contributes to glioma cell growth/survival through p53-independent functions.

JAZ has unique longer linker sequences between its zinc finger domains (Yang et al., Blood 108:4136-4145, 2006; Chen et al., Mol Cell Biol 2004:24: 6608-6619), which are likely to be as important as its zinc finger domains to JAZ's nuclear localization (Yang et al., J Biol Chem 1999:274: 27399-27406; Yang et al., Blood 108:4136-4145, 2006; Chen et al., Mol Cell Biol 2004:24: 6608-6619; Mendez-Vidal et al., Nucleic Acids Res 2002:30: 1991-1996; Moller et al., J Mol Biol 2005:351: 718-730; Yoon et al., Mol Cancer Ther 2003:2: 1171-1181). Molecular docking is a structure-based drug design approach that is useful for lead drug discovery. Targeting a JAZ structural binding pocket or unique linker sequences using a molecular docking approach will likely block JAZ's biological function and reduce neoplastic cell growth or survival. Using a molecular docking approach, a class of substituted diphenyl urea compounds was discovered that exhibits a strong neoplasia cytotoxic effect. Among the cytotoxic compounds described herein, J1 likely belongs to a microtubule-interacting class of compounds. J1 shares nearly identical NCU Yeast anti-cancer drug screen stage 1 results with the G2 checkpoint abrogator “UCN-01”. Therefore J1 likely interferes with cell cycle checkpoints and may have synergistic cytotoxic effects on glioma with temozolomide.

### Compounds of the Invention

Substituted diphenyl urea compounds were found to have a cytotoxic effect on neoplasias, such as gliomas, lung cancer cells, and colon cancer cells. Without wishing to be bound by theory, these compounds may be particularly effective against proliferating neoplastic cells that express increased levels of JAZ or that exhibit an altered JAZ subcellular localization. In one approach, cytotoxic compounds useful for the treatment of neoplasia are selected using a molecular docking program to identify compounds that are expected to bind to a JAZ structural binding pocket. Such compounds are sometimes referred to herein as “JAZ structural binding pocket” compounds. In certain embodiments, a compound of the invention can bind to JAZ and reduce JAZ biological activity and/or disrupt JAZ subcellular localization.

In certain embodiments, a compound of the invention can prevent, inhibit, or disrupt, or reduce by at least 10%, 25%, 50%, 75%, or 100% JAZ biological activity, JAZ expression, and/or disrupt JAZ subcellular localization proteins, e.g., by binding to a binding site in a JAZ structural binding pocket.

In certain embodiments, a compound of the invention is a small molecule having a molecular weight less than about 1000 daltons, less than 800, less than 600, less than 500, less than 400, or less than about 300 daltons. Examples of compounds of the invention include J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea), and pharmaceutically acceptable salts thereof.

The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like. The term “pharmaceutically acceptable salt” also refers to a salt prepared from a compound disclosed herein, e.g., a compound of J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea), or any other compound delineated herein, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

### In Silico Screening Methods and Systems

In another aspect, the invention provides a machine readable storage medium which comprises the structural coordinates of a JAZ polypeptide (e.g., JAZ structural binding pocket) binding site identified herein. This is the proposed binding site of J1. A storage medium encoded with these data is capable of displaying a three-dimensional graphical representation of a molecule or molecular complex which comprises such binding sites on a computer screen or similar viewing device.

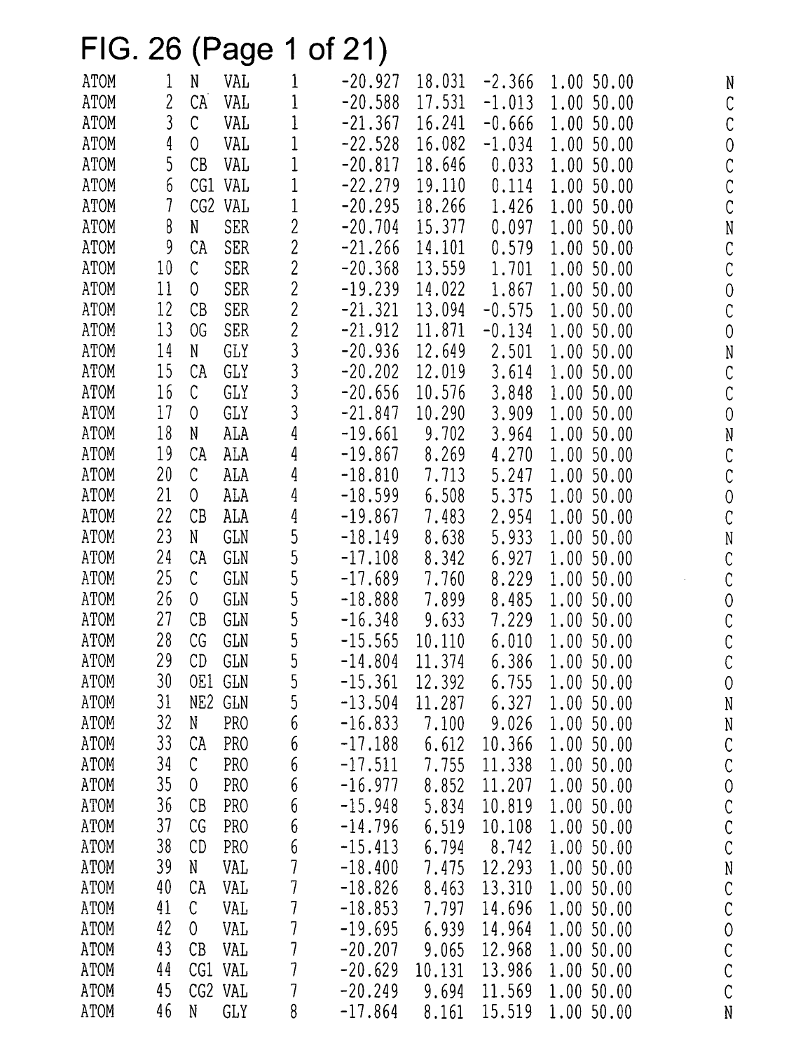

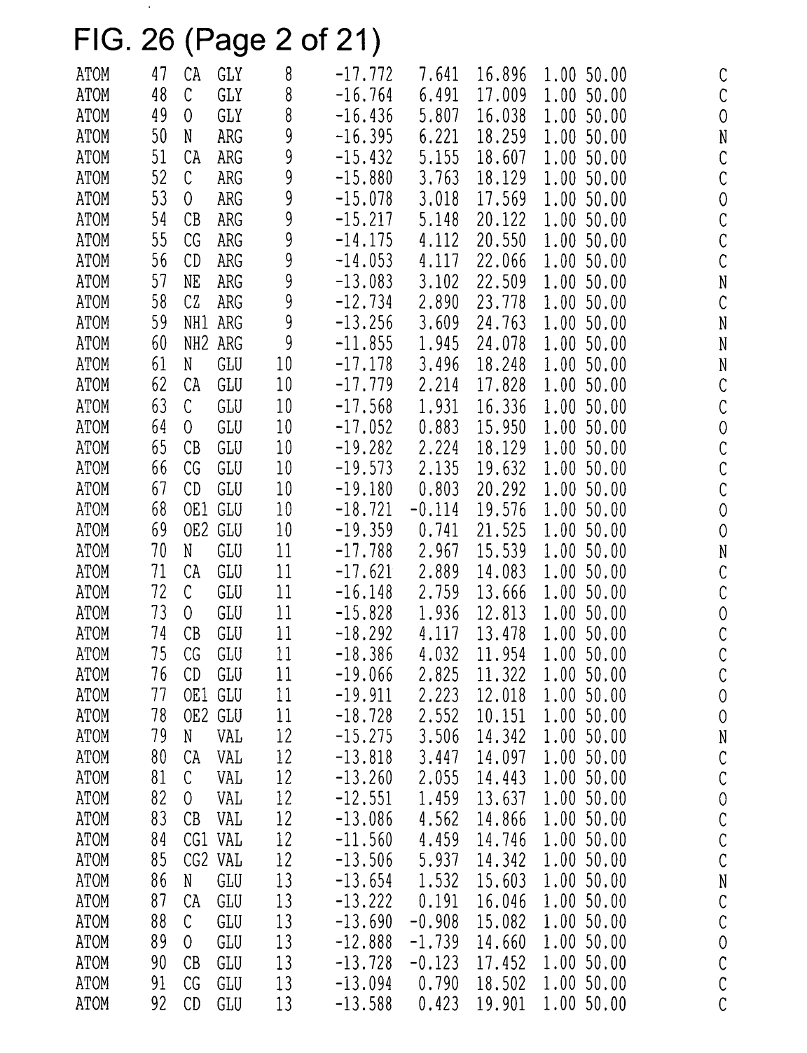

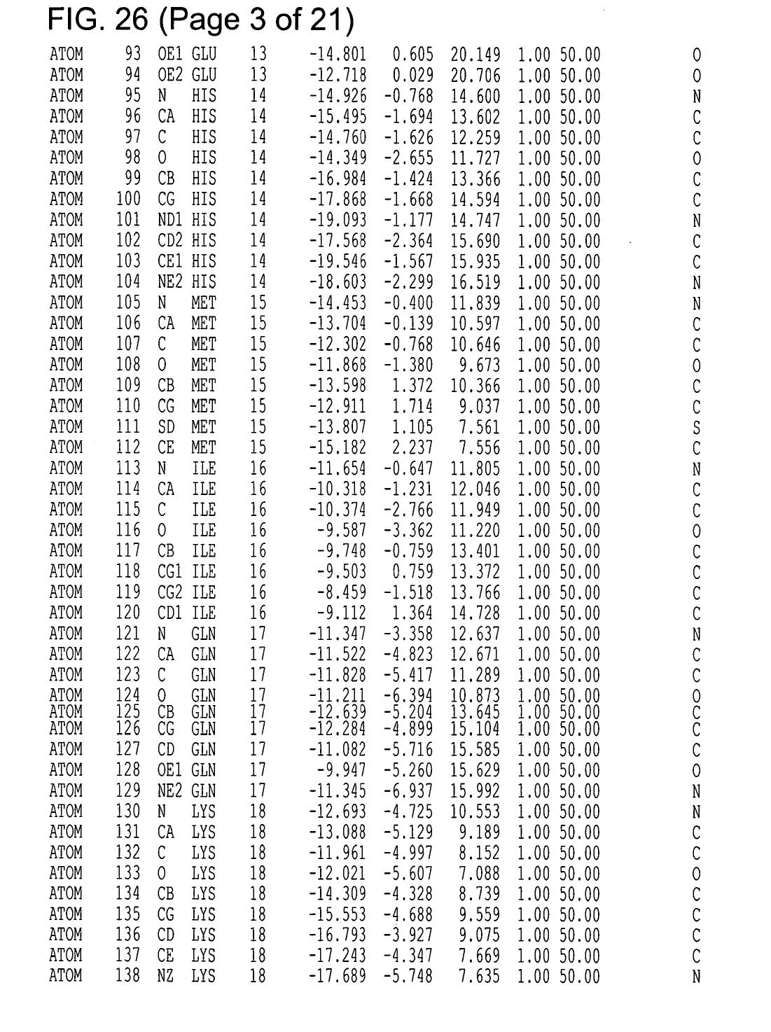

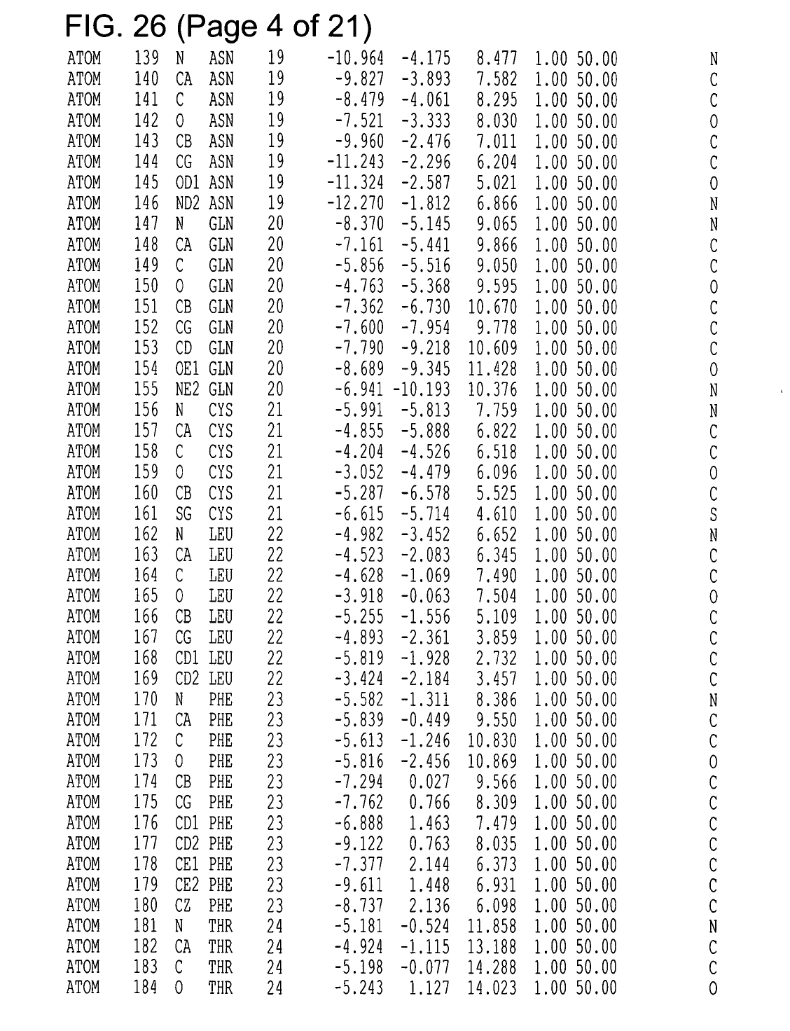

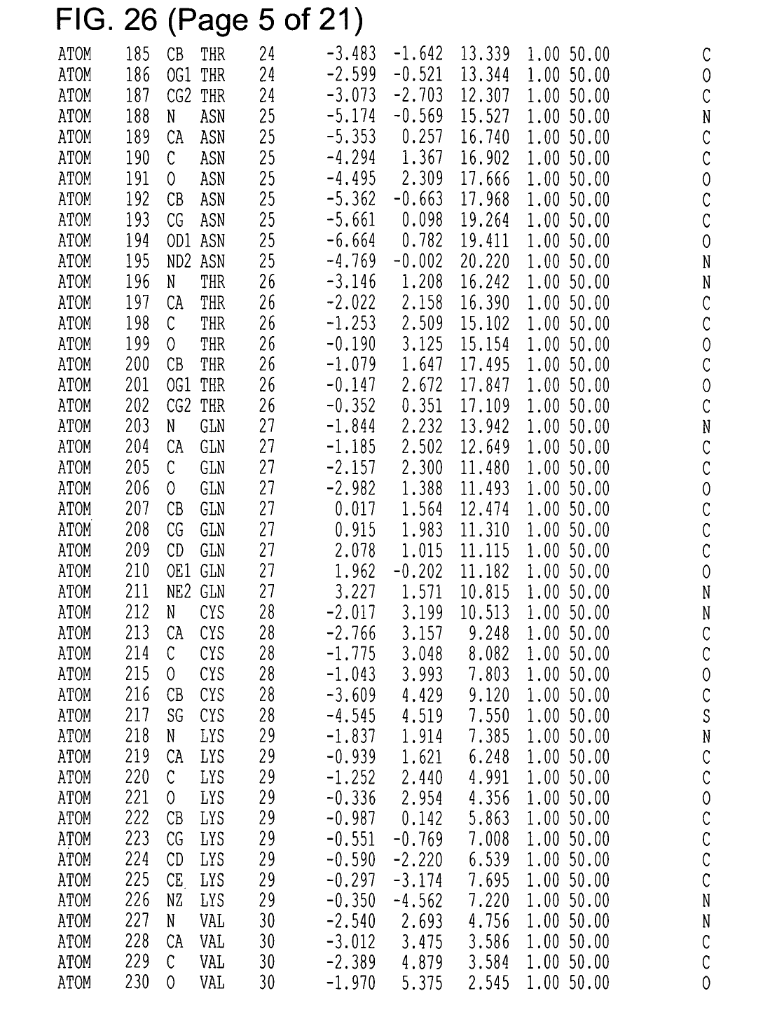

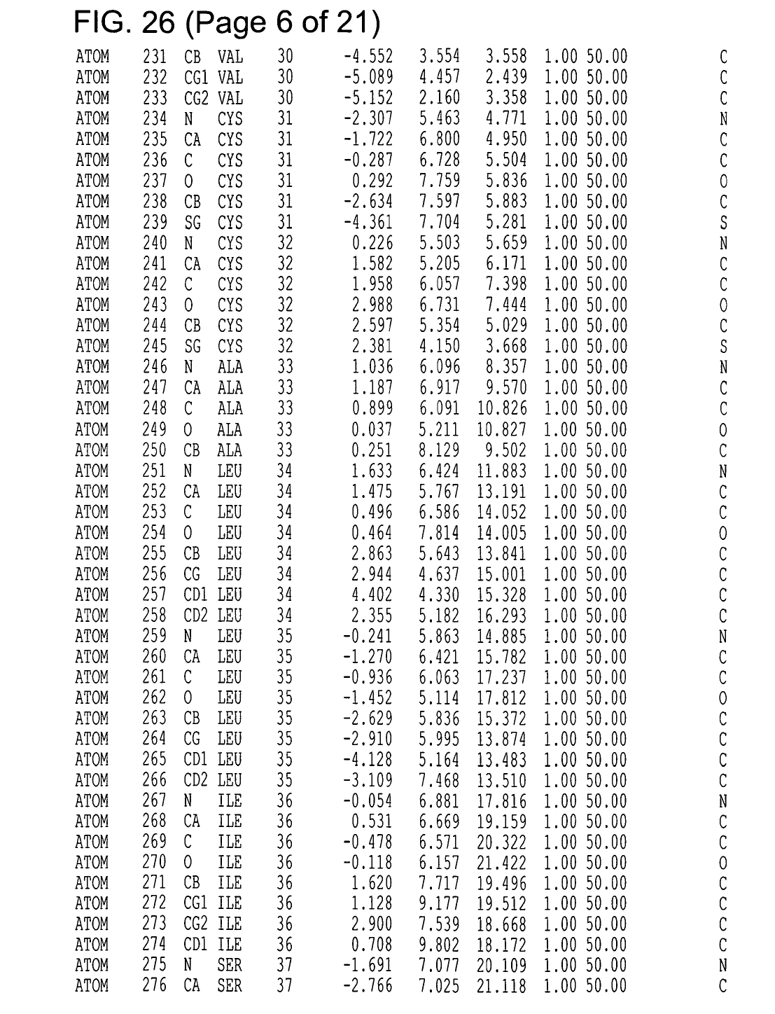

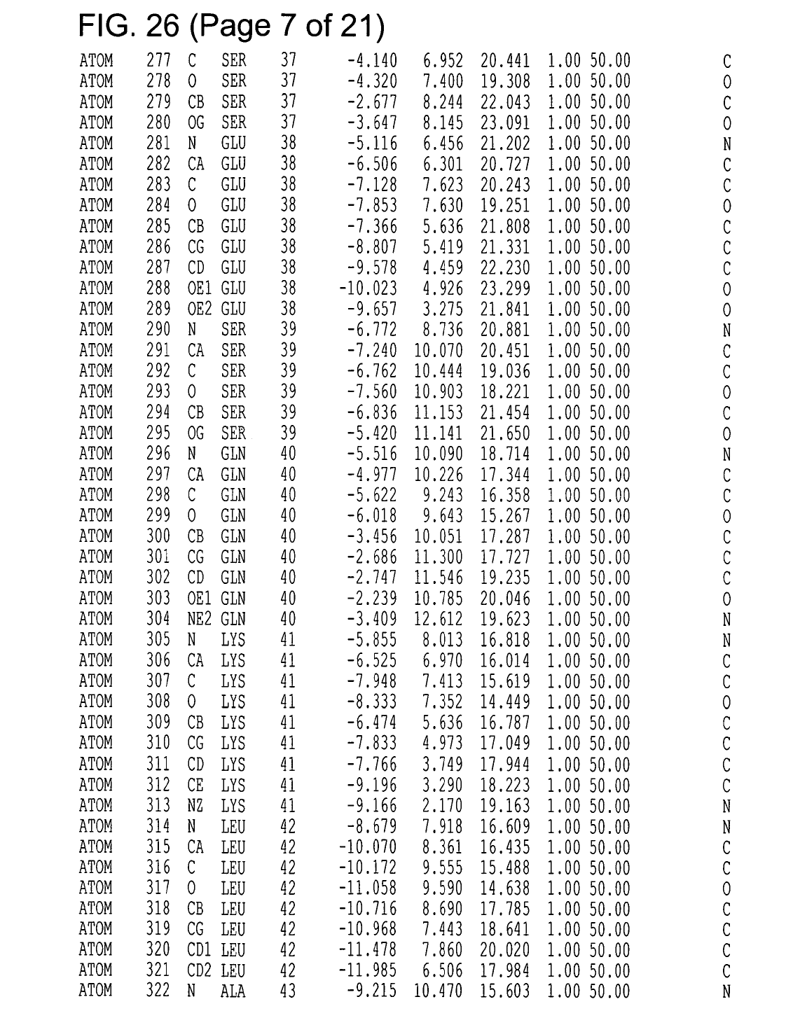

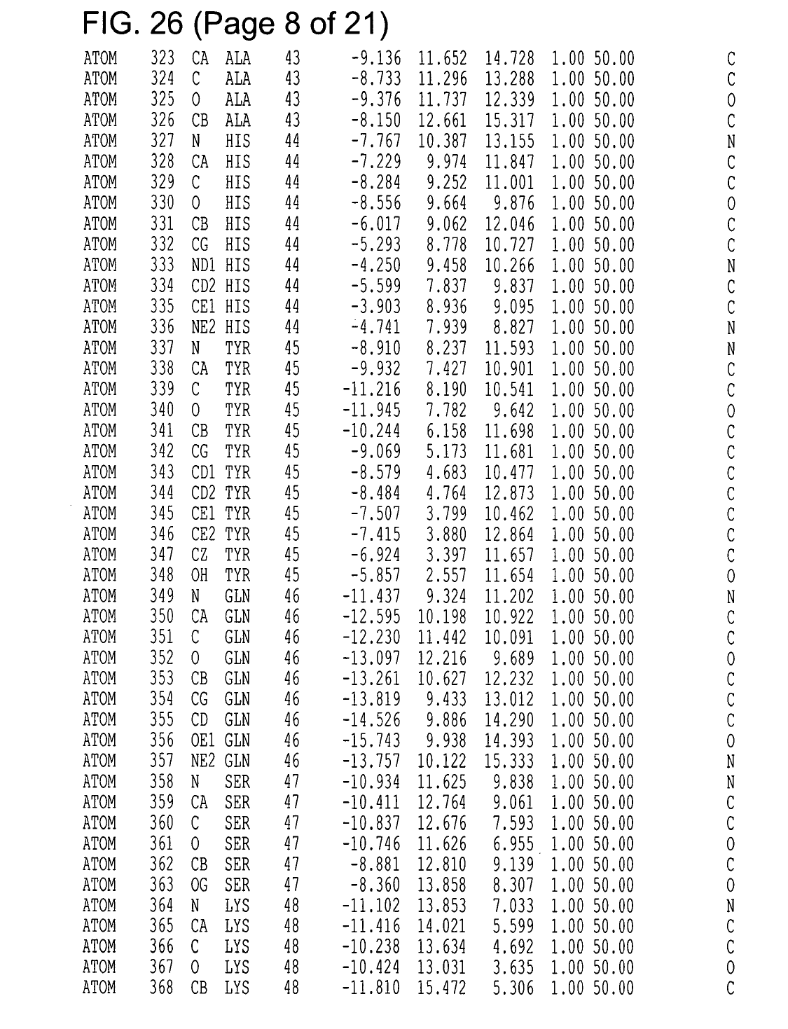

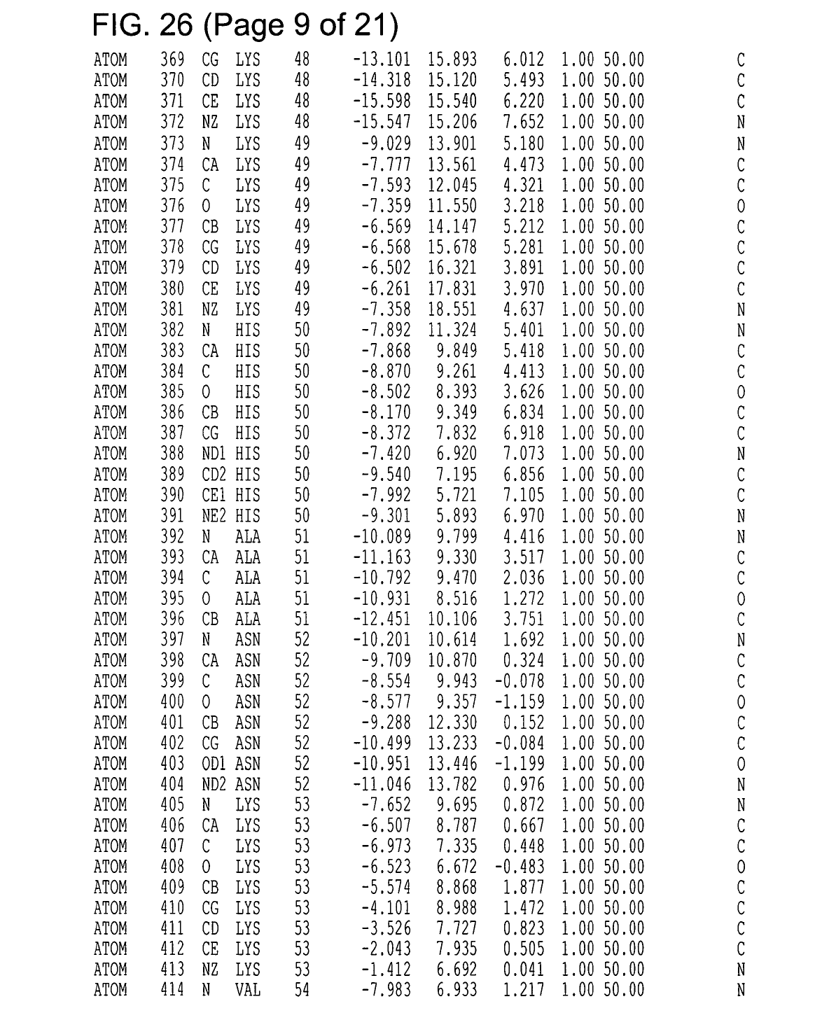

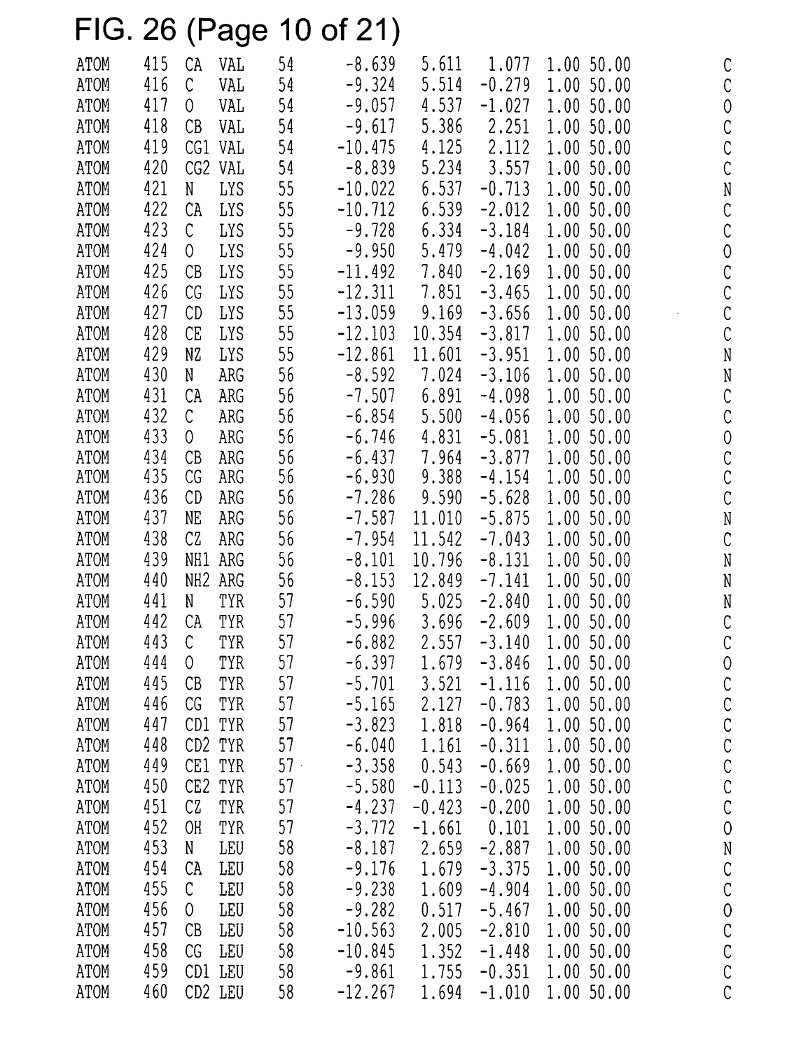

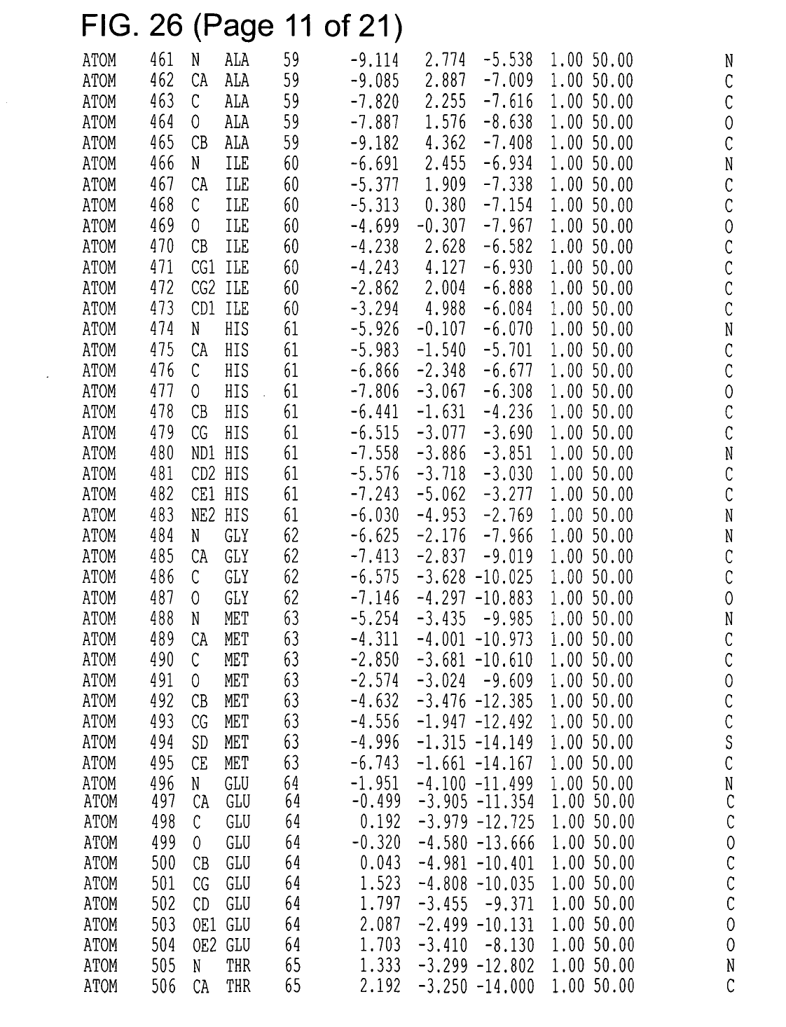

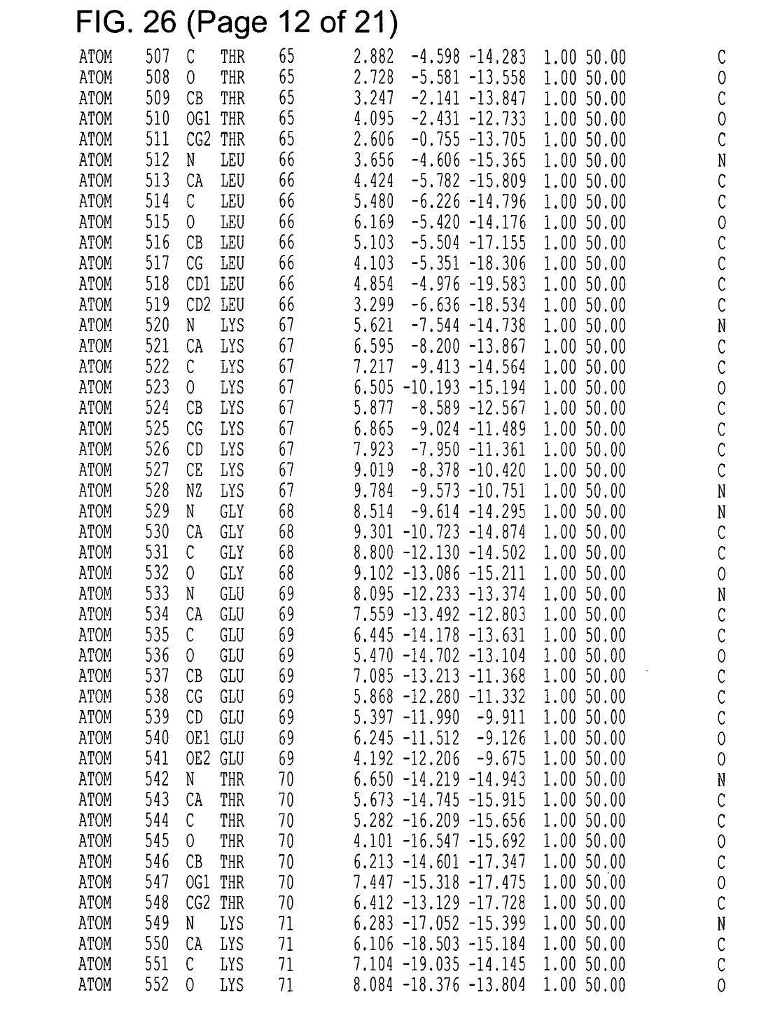

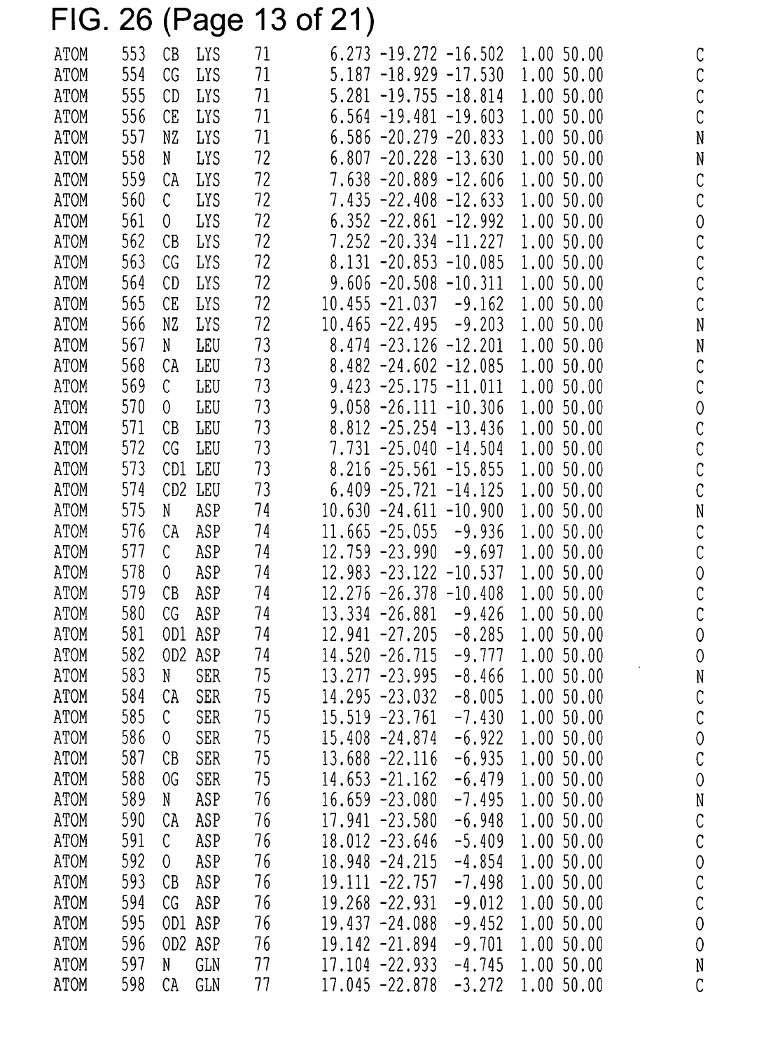

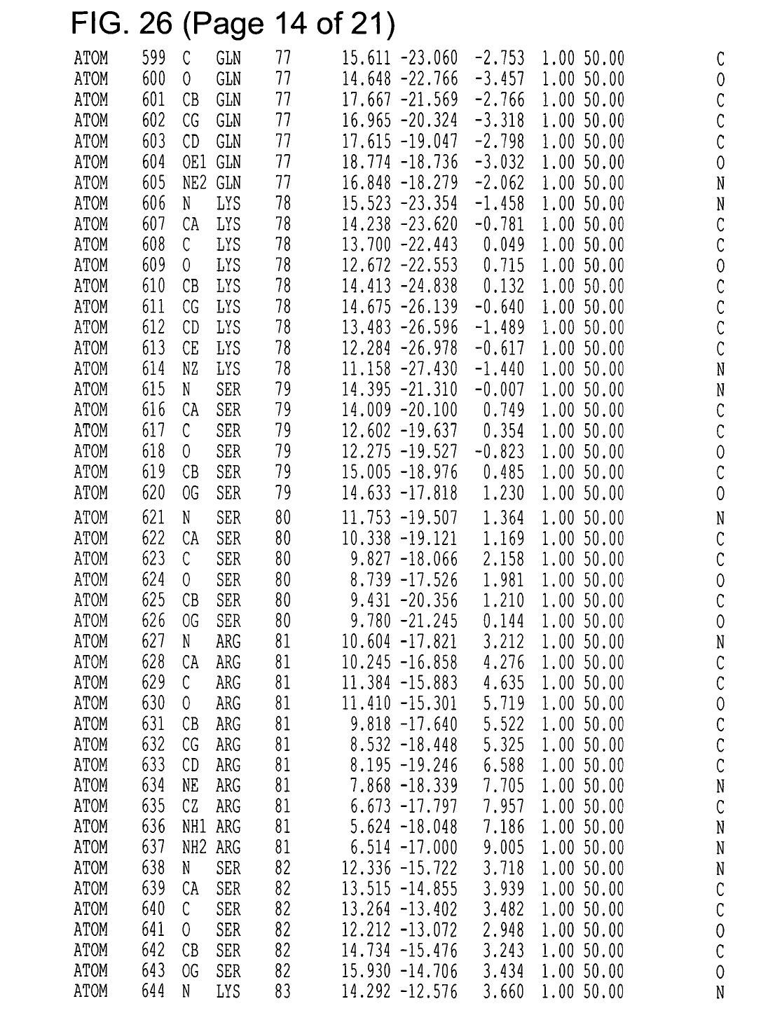

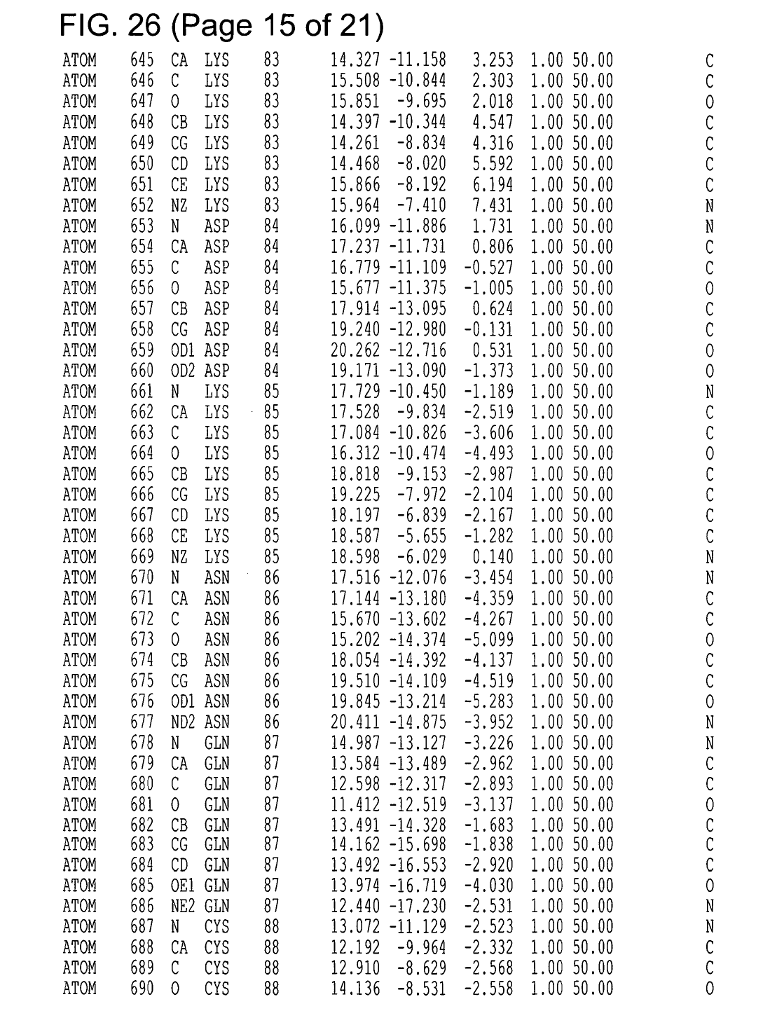

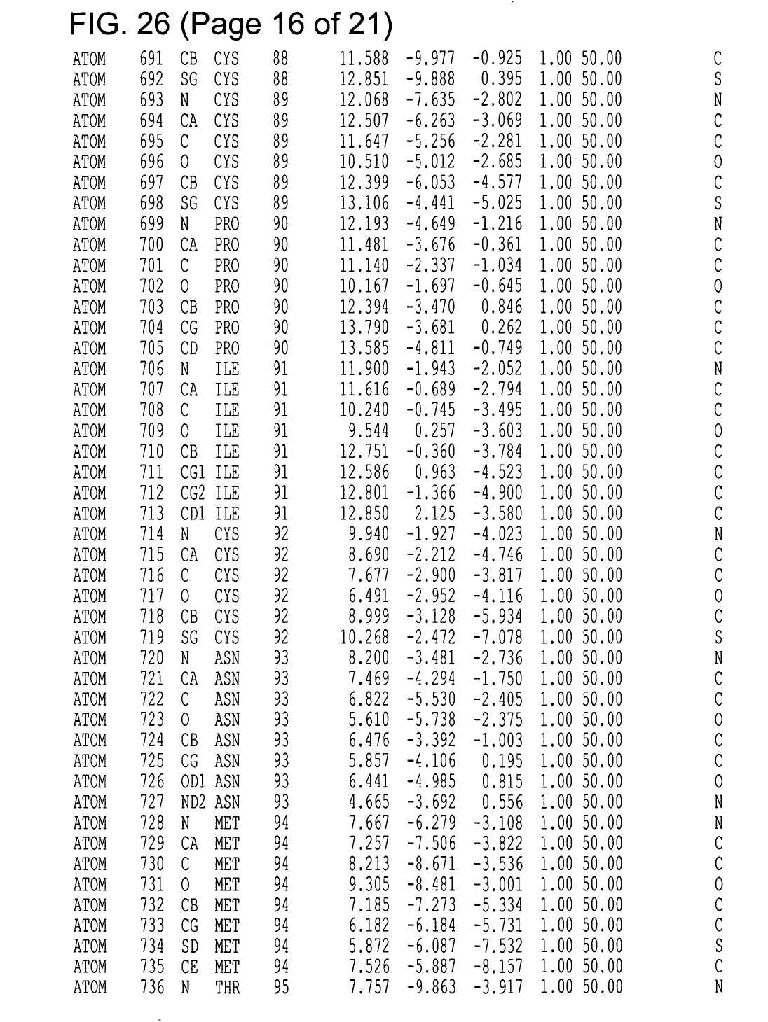

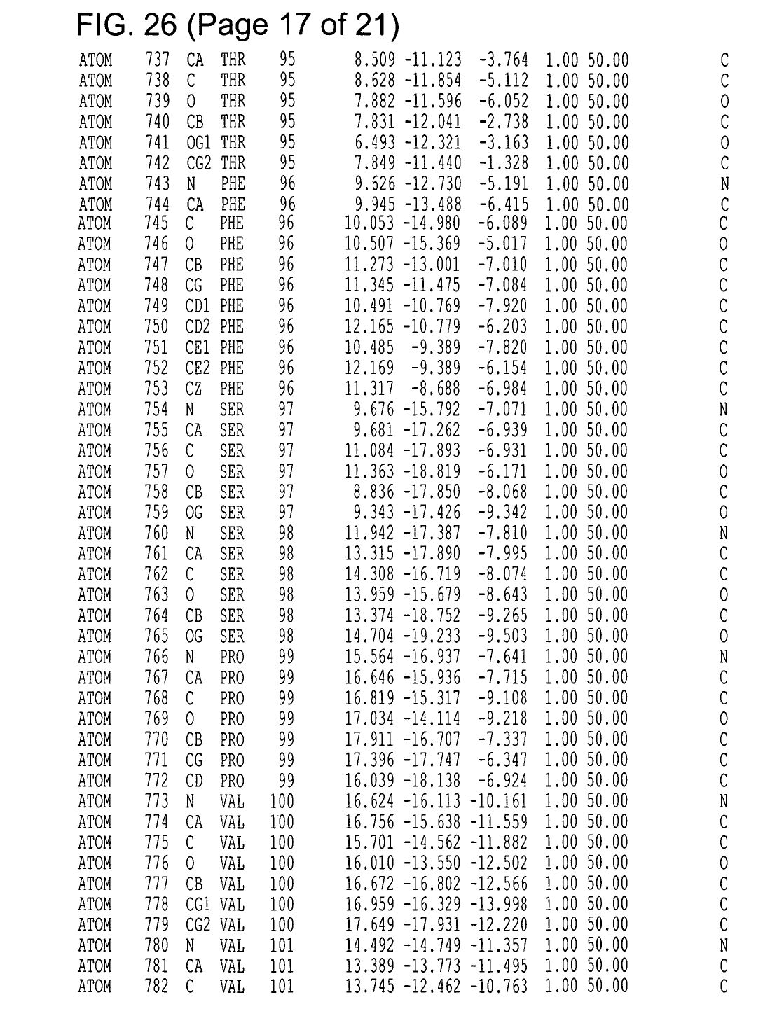

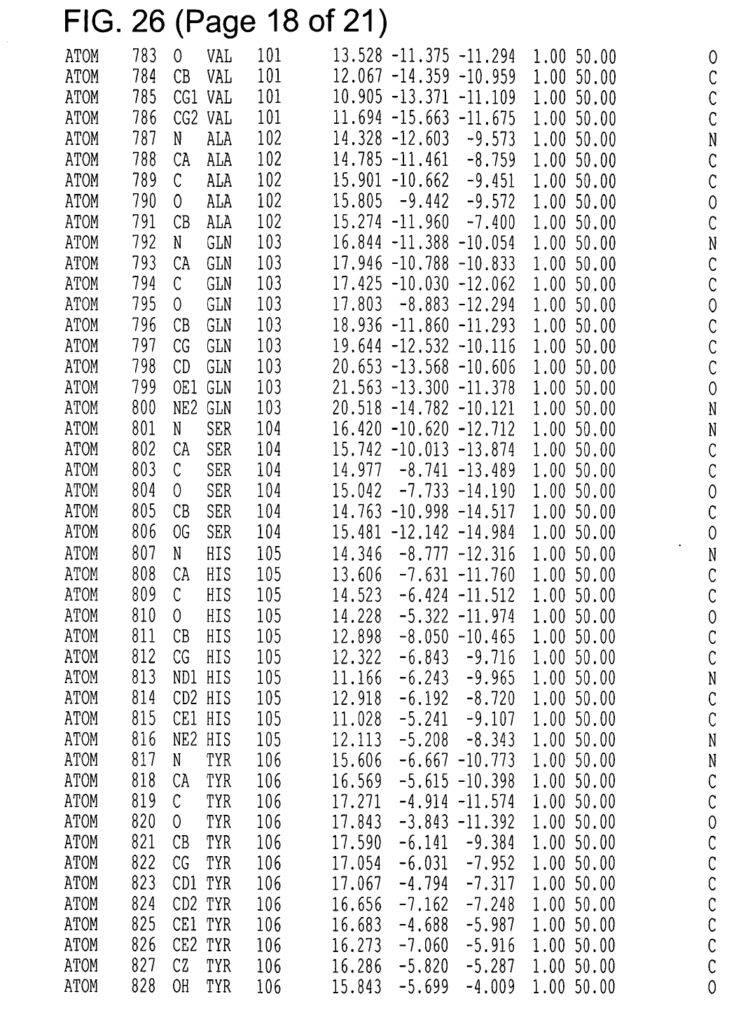

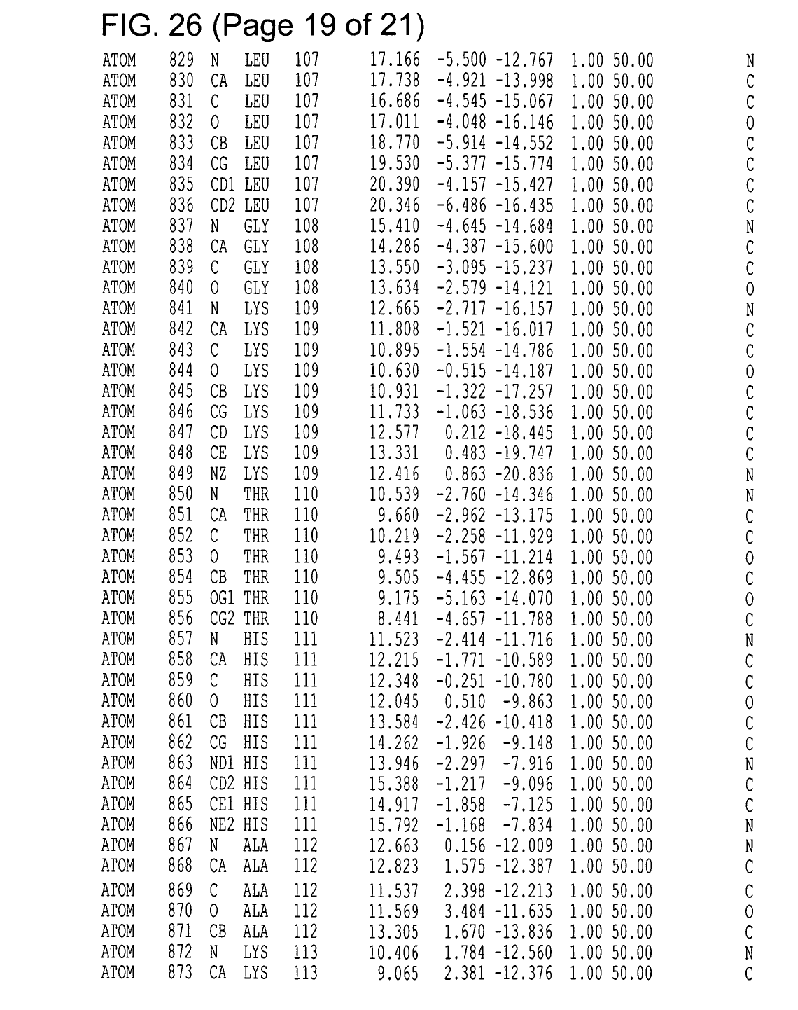

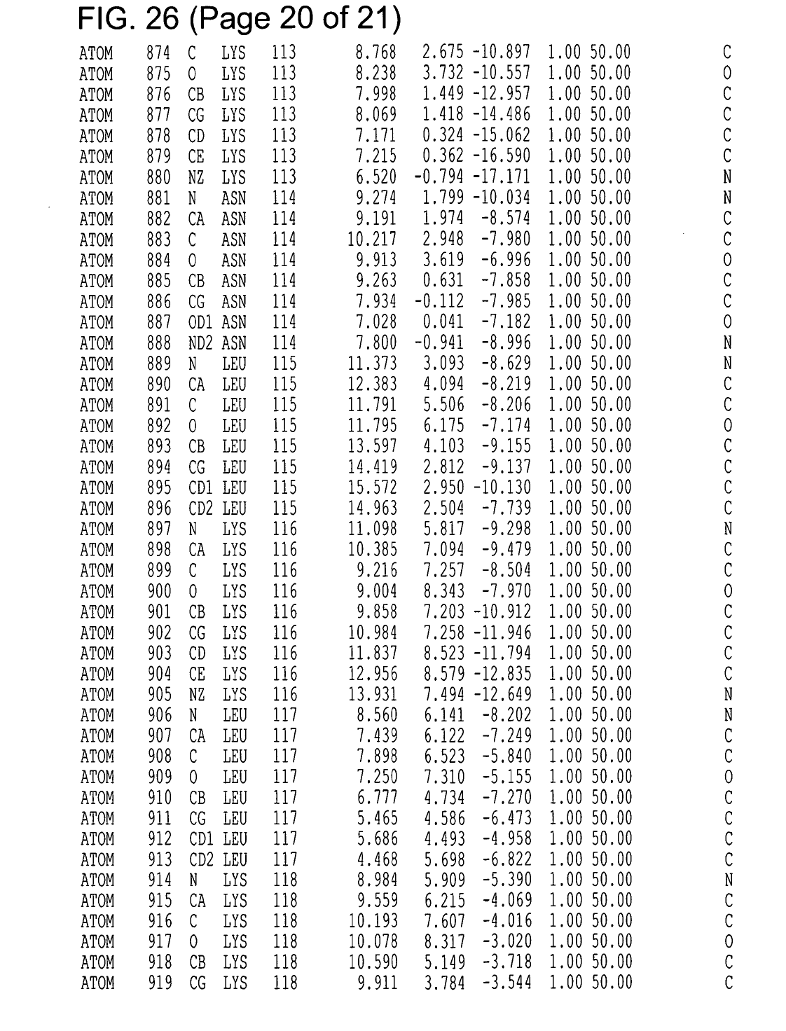

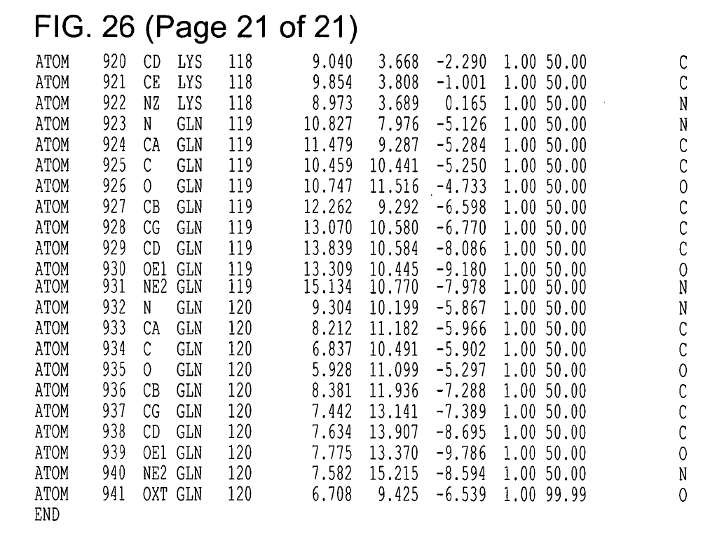

The National Cancer Institute/Developmental Therapeutics Program (NCI/DTP) maintains a repository of approximately 139 644 samples (the plated compound set) that are nonproprietary and offered to the extramural research community for the discovery and development of new agents for the treatment of cancer, AIDS, or opportunistic infections afflicting patients with cancer or AIDS. The three-dimensional coordinates for the NCI/DTP plated compound set was obtained in the MDL SD format and converted to the mol2 format by the DOCK utility program SDF2MOL2. These were used to identify agents that bind within the three-dimensional graphical representation of the JAZ binding site (FIG. 26).

The invention also provides methods for designing, evaluating and identifying compounds that bind to the aforementioned binding site. Such compounds are expected to be cytotoxic, to inhibit JAZ biological activity and/or to disrupt JAZ subcellular localization. The invention provides a computer for producing a) a three-dimensional representation of a molecule or molecular complex, wherein said molecule or molecular complex comprises a binding site; or b) a three-dimensional representation of a homologue of said molecule or molecular complex, wherein said homologue comprises a binding site that has a root mean square deviation from the backbone atoms of said amino acids of not more than about 2.0 (more preferably not more than 1.5) angstroms, wherein said computer comprises:

(i) a machine-readable data storage medium comprising a data storage material encoded with machine-readable data, wherein said data comprises the structure coordinates of amino acid residues in the JAZ structural binding pocket or other JAZ binding site;

(ii) a working memory for storing instructions for processing said machine-readable data;

(iii) a central-processing unit coupled to said working memory and to said machine-readable data storage medium for processing said machine readable data into said three-dimensional representation; and

(iv) a display coupled to said central-processing unit for displaying said three-dimensional representation.

Thus, the computer produces a three-dimensional graphical structure of a molecule or a molecular complex which comprises a binding site.

In another embodiment, the invention provides a computer for producing a three-dimensional representation of a molecule or molecular complex defined by structure coordinates of all of the JAZ amino acids, or a three-dimensional representation of a homologue of said molecule or molecular complex, wherein said homologue comprises a binding site that has a root mean square deviation from the backbone atoms of said amino acids of not more than 2.0 (more preferably not more than 1.5) angstroms

In exemplary embodiments, the computer or computer system can include components that are conventional in the art, e.g., as disclosed in U.S. Pat. Nos. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of this invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of this invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of this invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening (not shown) for receiving the spindle of a disk drive or other data storage device.

The magnetic domains of the medium are polarized or oriented so as to encode in a manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a binding site of a JAZ protein are expected to be toxic to neoplastic cells (e.g., glioma, lung cancer, colon cancer cells), to inhibit JAZ biological activity, and/or to disrupt JAZ subcellular localization. Such compounds are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a) a molecule or molecular complex comprising a binding site defined by structure coordinates of JAZ, as described herein, or b) a homologue of said molecule or molecular complex, wherein said homologue comprises a binding pocket that has a root mean square deviation from the backbone atoms of said amino acids of not more than 2.0 (more preferably 1.5) angstroms.

This method comprises the steps of:

i) employing computational means to perform a fitting operation between the chemical entity and a binding site of the JAZ polypeptide or fragment thereof or molecular complex; and

ii) analyzing the results of the fitting operation to quantify the association between the chemical entity and the binding pocket. This embodiment relates to evaluating the potential of a chemical entity to associate with or bind to a binding site of a JAZ polypeptide or fragment thereof.

The term “chemical entity”, as used herein, refers to chemical compounds, complexes of at least two chemical compounds, and fragments of such compounds or complexes.

In certain embodiments, the method evaluates the potential of a chemical entity to associate with a molecule or molecular complex defined by structure coordinates of all of the amino acids of JAZ protein, as described herein, or a homologue of said molecule or molecular complex having a root mean square deviation from the backbone atoms of said amino acids of not more than 2.0 (more preferably not more than 1.5) angstroms.

In a further embodiment, the structural coordinates one of the binding sites described herein can be utilized in a method for identifying an antagonist of a molecule comprising a JAZ binding site (e.g., a JAZ structural binding pocket). This method comprises the steps of:

a) using the atomic coordinates of JAZ; and

b) employing the three-dimensional structure to design or select the potential agonist or antagonist. One may obtain the compound by any means available. By “obtaining” is meant, for example, synthesizing, buying, or otherwise procuring the agonist or antagonist. If desired, the method further involves contacting the agonist or antagonist with a JAZ polypeptide or a fragment thereof to determine the ability of the potential agonist or antagonist to interact with the molecule. If desired, the method also further involves the step of contacting a neoplastic cell (e.g., glioma cell) with a JAZ binding compound and evaluating cytotoxicity, evaluating neoplastic cell proliferation, cell death, JAZ biological activity, JAZ expression, or JAZ subcellular localization.

In another embodiment, the invention provides a method for identifying a potential agonist or antagonist of JAZ polypeptide, the method comprising the steps of:

a) using the atomic coordinates of the JAZ polypeptide (e.g., JAZ structural binding pocket); and

b) employing the three-dimensional structure to design or select the potential agonist or antagonist.

The present inventors' elucidation of heretofore unidentified binding sites of JAZ polypeptides provides the necessary information for designing new chemical entities and compounds that may interact with JAZ proteins, in whole or in part, and may therefore modulate (e.g., inhibit) the activity of JAZ proteins.

The design of compounds that bind to a JAZ structural binding pocket sequence, that are cytotoxic to a neoplastic cell, that reduce JAZ expression or biological activity, or that disrupt JAZ subcellular localization, according to this invention generally involves consideration of several factors. In one embodiment, the compound physically and/or structurally associates with at least a fragment of a JAZ polypeptide, such as a binding site within a JAZ structural binding pocket sequence. Non-covalent molecular interactions important in this association include hydrogen bonding, van der Waals interactions, hydrophobic interactions and electrostatic interactions. Desirably, the compound assumes a conformation that allows it to associate with the JAZ binding site(s) directly. Although certain portions of the compound may not directly participate in these associations, those portions of the entity may still influence the overall conformation of the molecule. This, in turn, may have a significant impact on the compound's potency. Such conformational requirements include the overall three-dimensional structure and orientation of the chemical compound in relation to all or a portion of the binding site, or the spacing between functional groups comprising several chemical compound that directly interact with the binding site or a homologue thereof.

The potential inhibitory or binding effect of a chemical compound on a JAZ binding site may be analyzed prior to its actual synthesis and testing by the use of computer modeling techniques. If the theoretical structure of the given compound suggests insufficient interaction and association between it and the target binding site, testing of the compound is obviated. However, if computer modeling indicates a strong interaction, the molecule is synthesized and tested for its ability to bind a JAZ structural binding pocket sequence or to test its biological activity by assaying for example, cytotoxicity in a neoplastic cell, by assaying a reduction in JAZ expression or biological activity, or by assaying JAZ subcellular localization. Candidate compounds may be computationally evaluated by means of a series of steps in which chemical entities or fragments are screened and selected for their ability to associate with the JAZ structural binding pocket.

One skilled in the art may use one of several methods to screen chemical compounds, or fragments for their ability to associate with a JAZ binding site. This process may begin by visual inspection of, for example, a JAZ binding site on the computer screen based on the a JAZ structure coordinates described herein, or other coordinates which define a similar shape generated from the machine-readable storage medium. Selected fragments or chemical compounds are then positioned in a variety of orientations, or docked, within that binding site as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding site.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)). Once a compound has been designed or selected, the efficiency with which that entity may bind to a binding site may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art.

Another technique involves the in silico screening of virtual libraries of compounds, e.g., as described herein (see, e.g., Examples). Many thousands of compounds can be rapidly screened and the best virtual compounds can be selected for further screening (e.g., by synthesis and in vitro or in vivo testing). Small molecule databases can be screened for chemical entities or compounds that can bind, in whole or in part, to a JAZ binding site. In this screening, the quality of fit of such entities to the binding site may be judged either by shape complementarity or by estimated interaction energy.

A computer for producing a three-dimensional representation of

a) a molecule or molecular complex, wherein said molecule or molecular complex comprises a structural binding pocket of a JAZ polypeptide defined by structure coordinates of amino acid residues in the structural binding pocket sequence of a JAZ polypeptide; or

b) a three-dimensional representation of a homologue of said molecule or molecular complex, wherein said homologue comprises a binding site that has a root mean square deviation from the backbone atoms of said amino acids of not more than about 2.0 (more preferably not more than 1.5) angstroms, wherein said computer comprises:

(i) a machine-readable data storage medium comprising a data storage material encoded with machine-readable data, wherein said data comprises the structure coordinates of structure coordinates of amino acid residues in the structural binding pocket sequence of a JAZ polypeptide;

(ii) a working memory for storing instructions for processing said machine-readable data;

(iii) a central-processing unit coupled to said working memory and to said machine-readable data storage medium for processing said machine readable data into said three-dimensional representation; and

(iv) a display coupled to said central-processing unit for displaying said three-dimensional representation. As described in the Examples, compounds identified using in silico methods may optionally be tested in vitro or in vivo, for example, using the “Additional Screening Methods” described below, or any other method known in the art.

### Additional Screening Methods

As described above, the invention provides specific examples of chemical compounds, including J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea, as well as other substituted diphenyl urea compounds that are cytotoxic to neoplastic cells. However, the invention is not so limited. The invention further provides a simple means for identifying agents (including nucleic acids, peptides, small molecule inhibitors, and mimetics) that are capable of binding to a JAZ polypeptide, that are cytotoxic to a neoplastic cell, that reduce JAZ expression or biological activity, or that disrupt JAZ subcellular localization. Such compounds are also expected to be useful for the treatment or prevention of a neoplasia (e.g., glioma).

In particular, based in part on the discovery that increased expression of JAZ contributes to glioma development and aggressiveness, agents that reduce the expression of a JAZ nucleic acid molecule or polypeptide are likely useful as therapeutics for the treatment or prevention of a neoplasia. Accordingly, the invention provides agents, such as inhibitory nucleic acid molecules (e.g., antisense, siRNA, shRNA) that reduce the expression of a JAZ polypeptide. In one approach, the ability of inhibitory nucleic acid molecules to silence JAZ gene expression is assayed in a glioma cell line. In particular, embodiments, the effect of a compound or other agent of the invention is analyzed by assaying p53 regulation, cell cycle dynamics, cell invasion, and cell death. In another approach, agents and compounds of the invention are assayed for their effect on glioma growth in an intracranial U87 transplant mouse glioma model or any other available glioma animal models. Agents and compounds of the invention that reduce the growth, proliferation, or invasiveness of a neoplasia are identified as useful for the treatment or prevention of a neoplasia.

Virtually any agent that specifically binds to a JAZ polypeptide or that modulates JAZ expression or biological activity may be employed in the methods of the invention. Methods of the invention are useful for the high-throughput low-cost screening of candidate agents that reduce, slow, or stabilize the growth or proliferation of a neoplasia. A candidate agent that specifically binds to JAZ is then isolated and tested for activity in an in vitro assay or in vivo assay for its ability to reduce neoplastic cell proliferation, reduce glioma cell invasion, and/or increase neoplastic cell death. One skilled in the art appreciates that the effects of a candidate agent on a cell is typically compared to a corresponding control cell not contacted with the candidate agent. Thus, the screening methods include comparing the proliferation of a neoplastic cell contacted by a candidate agent to the proliferation of an untreated control cell.

In other embodiments, the expression or activity of JAZ in a cell treated with a candidate agent is compared to untreated control samples to identify a candidate compound that decreases the expression or biological activity of a JAZ polypeptide in the contacted cell. Polypeptide expression or activity can be compared by procedures well known in the art, such as Western blotting, flow cytometry, immunocytochemistry, binding to magnetic and/or JAZ-specific antibody-coated beads, in situ hybridization, fluorescence in situ hybridization (FISH), ELISA, microarray analysis, RT-PCR, Northern blotting, or colorimetric assays, such as the Bradford Assay and Lowry Assay.

In one working example, one or more candidate agents are added at varying concentrations to the culture medium containing a neoplastic cell. An agent that reduces the expression of a JAZ polypeptide expressed in the cell is considered useful in the invention; such an agent may be used, for example, as a therapeutic to prevent, delay, ameliorate, stabilize, or treat a neoplasia. Once identified, agents of the invention (e.g., agents that specifically bind to and/or antagonize JAZ) may be used to treat a neoplasia. An agent identified according to a method of the invention is locally or systemically delivered to treat a neoplasia in situ.

In one embodiment, the effect of a candidate agent may, in the alternative, be measured at the level of JAZ polypeptide production using the same general approach and standard immunological techniques, such as Western blotting or immunoprecipitation with an antibody specific for JAZ. For example, immunoassays may be used to detect or monitor the expression of JAZ in a neoplastic cell. In one embodiment, the invention identifies a polyclonal or monoclonal antibody (produced as described herein) that is capable of binding to and blocking the biological activity or disrupting the subcellular localization of a JAZ polypeptide. A compound that disrupts the subcellular localization, or reduces the expression or activity of a JAZ polypeptide is considered particularly useful. Again, such an agent may be used, for example, as a therapeutic to prevent or treat a neoplasia.

Alternatively, or in addition, candidate compounds may be identified by first assaying those that specifically bind to and antagonize a JAZ polypeptide of the invention and subsequently testing their effect on neoplastic cells as described in the Examples. In one embodiment, the efficacy of a candidate agent is dependent upon its ability to interact with the JAZ polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate neoplastic cell proliferation may be assayed by any standard assays (e.g., those described herein). In one embodiment, division of neoplastic cells is determined by assaying BrdU incorporation using flow cytometry analysis. In another embodiment, JAZ expression is monitored immunohistochemically.

Potential JAZ antagonists include organic molecules, peptides, peptide mimetics, polypeptides, nucleic acid ligands, aptamers, and antibodies that bind to a JAZ polypeptide and reduce its activity. In one particular example, a candidate compound that binds to a JAZ polypeptide may be identified using a chromatography-based technique. For example, a recombinant JAZ polypeptide of the invention may be purified by standard techniques from cells engineered to express the polypeptide, or may be chemically synthesized, once purified the peptide is immobilized on a column. A solution of candidate agents is then passed through the column, and an agent that specifically binds the JAZ polypeptide or a fragment thereof is identified on the basis of its ability to bind to JAZ polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to reduce neoplastic cell proliferation or viability. Agents isolated by this approach may also be used, for example, as therapeutics to treat or prevent a neoplasia. Compounds that are identified as binding to a JAZ polypeptide with an affinity constant less than or equal to 1 nM, 5 nM, 10 nM, 100 nM, 1 μM or 10 μM are considered particularly useful in the invention.

### Test Compounds and Extracts

In general, JAZ antagonists (e.g., agents that specifically bind and reduce the activity of a JAZ polypeptide) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of a neoplasia. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have JAZ binding activity further fractionation of the positive lead extract is necessary to isolate molecular constituents responsible for the observed effect. Thus, the goal of the extraction, fractionation, and purification process is the careful characterization and identification of a chemical entity within the crude extract that reduces neoplastic cell proliferation or viability. Methods of fractionation and purification of such heterogenous extracts are known in the art. If desired, compounds shown to be useful as therapeutics are chemically modified according to methods known in the art.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neoplastic disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which neoplasia may be implicated.

In one embodiment, the invention provides a method of monitoring treatment progress. The method includes the step of determining a level of diagnostic marker (Marker) (e.g., JAZ, p53, or any target delineated herein modulated by a compound herein, a protein or indicator thereof, etc.) or diagnostic measurement (e.g., screen, assay) in a subject suffering from or susceptible to a disorder or symptoms thereof associated with neoplasia, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Polynucleotide Therapy

Polynucleotide therapy featuring a polynucleotide encoding a CHOP or ATF-4 protein, variant, or fragment thereof is another therapeutic approach for treating a neoplastic disease. Expression of such proteins in a neoplastic cell is expected to reduce survival of the cell, for example, by increasing cell death. Such nucleic acid molecules can be delivered to cells of a subject having a neoplasia. The nucleic acid molecules must be delivered to the cells of a subject in a form in which they can be taken up so that therapeutically effective levels of a CHOP or ATF-4 protein or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a CHOP or ATF-4 protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer a CHOP or ATF-4 polynucleotide systemically.

Non-viral approaches can also be employed for the introduction of therapeutic to a cell of a patient requiring inhibition of a neoplasia or induction of cell death in a neoplasia. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant a CHOP or ATF-4 protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Pharmaceutical Therapeutics

In other embodiments, agents discovered to have medicinal value using the methods described herein are useful as a drug or as information for structural modification of existing compounds, e.g., by rational drug design. Such methods are useful for screening agents having an effect on a neoplasia.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. A compound is administered at a dosage that is cytotoxic to a neoplastic cell, that reduces JAZ expression or biological activity, or that reduces the proliferation, survival, or invasiveness of a neoplastic cell as determined by a method known to one skilled in the art, or using any that assay that measures the expression or the biological activity of a JAZ polypeptide.

### Formulation of Pharmaceutical Compositions

The administration of a compound for the treatment of a neoplasia may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other embodiments, it is envisaged that doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutaminine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active anti-neoplasia therapeutic substance). The coating may be applied on the solid dosage form in a similar manner as that described in Encyclopedia of Pharmaceutical Technology, supra.

At least two anti-neoplasia therapeutics may be mixed together in the tablet, or may be partitioned. In one example, the first active anti-neoplasia therapeutic is contained on the inside of the tablet, and the second active anti-neoplasia therapeutic is on the outside, such that a substantial portion of the second anti-neoplasia therapeutic is released prior to the release of the first anti-neoplasia therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-neoplasia therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, an anti-neoplasia therapeutic may be administered in combination with any other standard anti-neoplasia therapy; such methods are known to the skilled artisan and described in Remington's Pharmaceutical Sciences by E. W. Martin. If desired, agents of the invention (e.g., J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea) are administered in combination with any conventional anti-neoplastic therapy, including but not limited to, surgery, radiation therapy, or chemotherapy. In one preferred embodiment, an agent of the invention is administered in combination with temozolomide.

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### JAZ Expression was Increased in Glioma

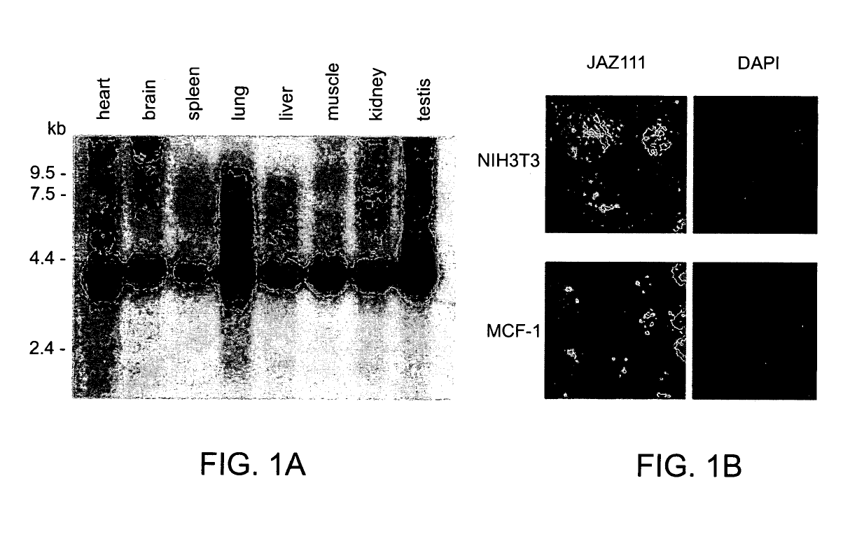

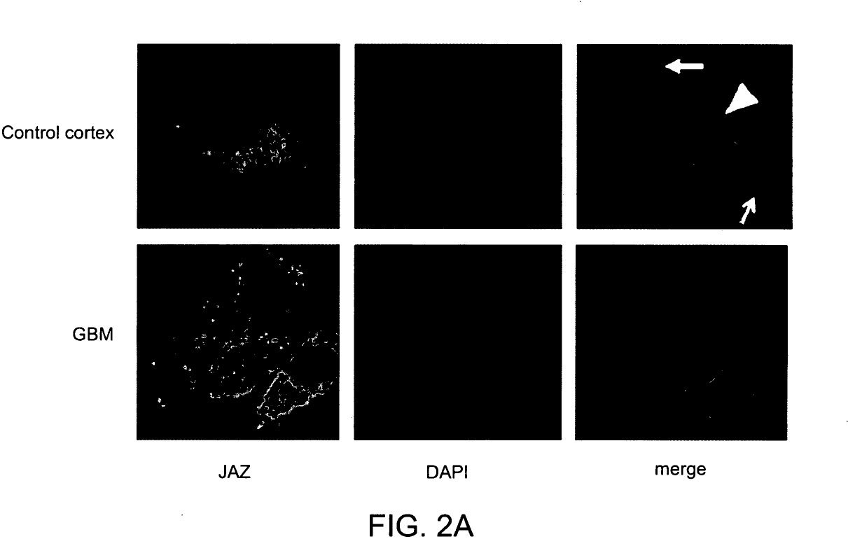

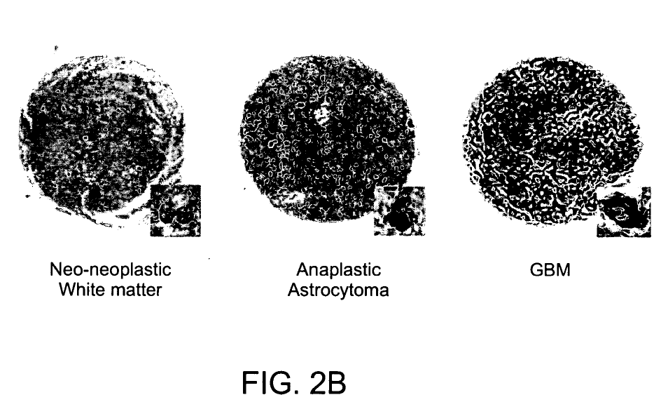

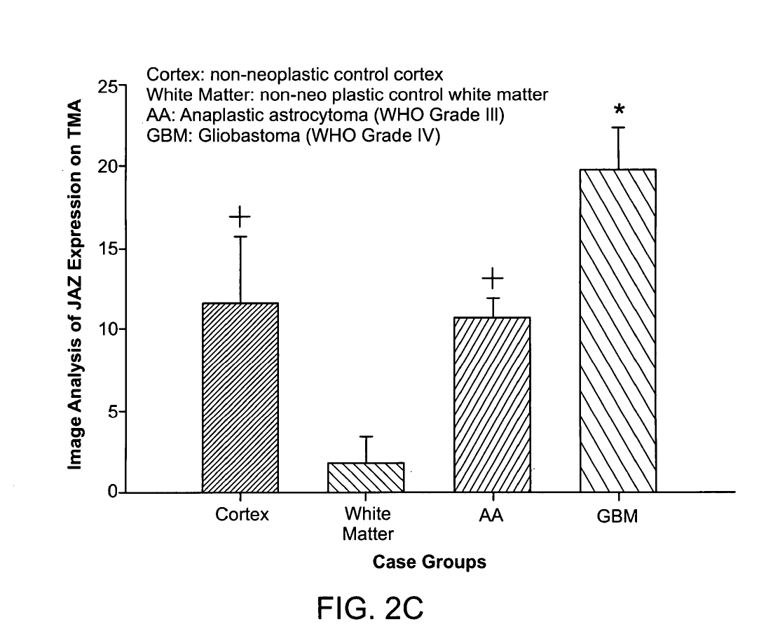

JAZ is a unique dsRNA binding nuclear/nucleolar zinc finger protein that is expressed in a variety of tissues (FIG. 1A). The JAZ polypeptide is a nuclear/nucleolar protein in cells in vitro (FIG. 1B). Immunofluorescence (IF) and immunohistochemistry (IHC) was used to detect the distribution of JAZ in human brain and glioma tissues. Immunofluorescence analysis detected JAZ in normal cortical neurons and glioblastoma tumor cells with a predominantly cytoplasmic distribution (FIG. 2 A). Normal astrocytes and oligodendrocytes had absent or weak anti-JAZ antibody reactivity (FIG. 2 A and Table 1).

TABLE 1
Immunohistochemistry results of JAZ, p53, and Ki67 in human glioma tissue microarray

| | Number | | | | |
|---|---|---|---|---|---|
| Tissue type | of cases | JAZ Expression by IHC ( ) | | P53 Expression by IHC (#) | Ki-67 (Mean) |
| Non-neoplastic (negative control) | 7 | Neurons | 7/7 strong | | 0% |
| | | Astrocytes | 3/7 weak | 7/7 absent | |
| | | | 4/7 absent | | |
| | | Oligos | 1/7 moderate | | |
| | | | 3/7 weak | | |
| | | | 3/7 absent | | |

| | | | JAZ Expression by IHC in | P53 Expression by IHC in | |
|---|---|---|---|---|---|
| | | | Neoplastic Cells ( ) | Neoplastic Cells (#) | |
| WHO Grade IV | Glioblastoma Multiforme | 13 | 11 strong | 10 strong | 10% |
| Tumor (1) | | | | 1 weak | |
| | | | 2 moderate | 2 strong | 4.5% |
| WHO Grade III | Anaplastic Astrocytoma | 5 | 5 strong | 4 strong | 9.7% |
| Tumor (1) | | | | 1 weak | |
| | Anaplastic Oligoastrocytoma | 1 | 1 strong | 1 strong | 2.5% |
| | Anaplastic Oligodendroglioma | 11 | 9 strong | 3 strong | 11.7% |
| | | | | 5 moderate | |
| | | | | 1 weak | |
| | | | 2 moderate | 1 strong | 14% |
| | | | | 1 weak | |

| | | | | |
|---|---|---|---|---|
| All tumors | 30 | 26 (87%) strong | 21 (70%) strong | 10.5% |
| | | 4 (13%) moderate | 5 (16.7%) moderate | |
| | | | 4 (13.3%) weak | |

.: JAZ IHC Criteria: Strong: over 70% of cells have JAZ staining; moderate: between 30% to 70% of tumor cells have JAZ staining; weak: less than 30% of cells have JAZ staining; absent: 0% of cells have JAZ staining.
#: P53 IHC Criteria: Strong: over 40% of cells have p53 staining; moderate: between 10% to 40% of tumor cells have p53 staining; weak: less than 10% of cells have p53 staining; absent: 0% of cells have p53 staining.

A semi-quantitative analysis of JAZ immunohistochemistry of glioma tissue-microarray (TMA) slides indicated that JAZ reactivity was increased in gliomas. Interestingly, glioblastomas (GBM) had the highest levels of JAZ protein (FIGS. 2B and 2C). Increased JAZ expression in glioma correlated with increased p53 expression, as well as increased ki-67 nuclear labeling (Table 1).

### Example 2

### Nuclear JAZ Expression was Increased in Glioblastoma Pseudopalisading Tumor Cells as Well as Glioma Cell Lines

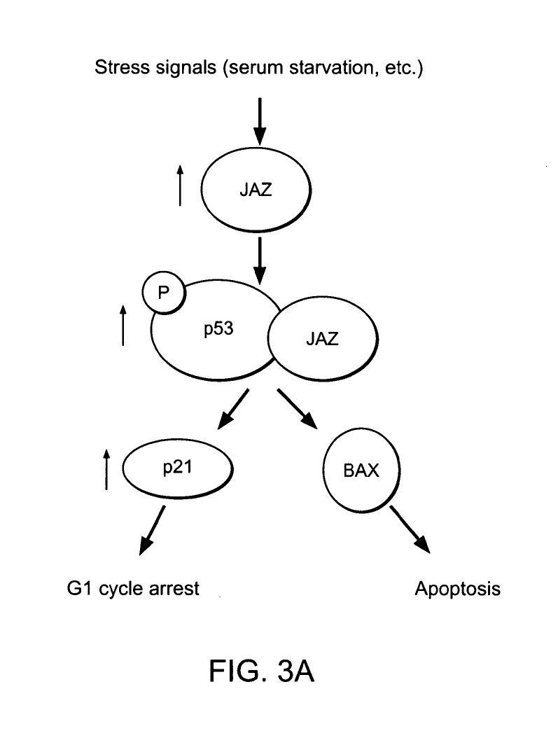

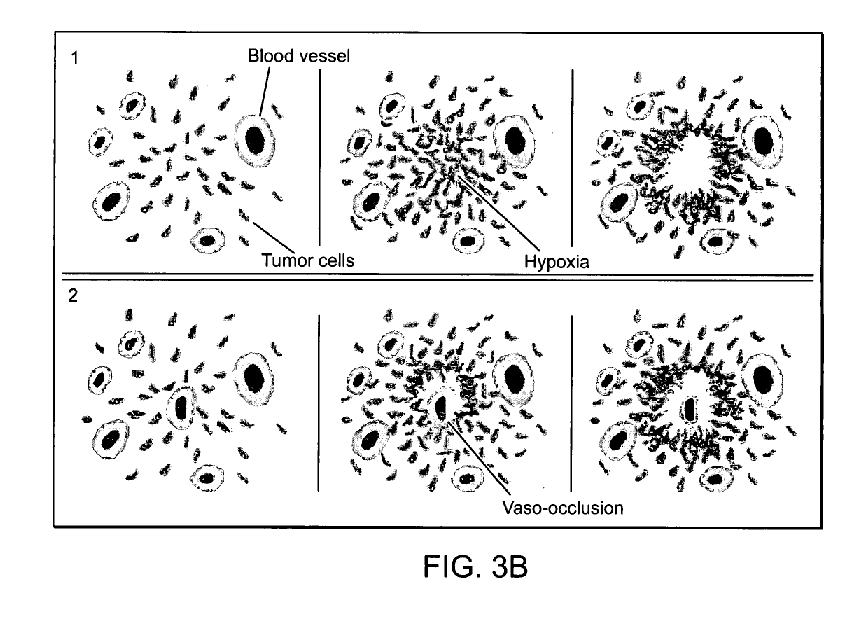

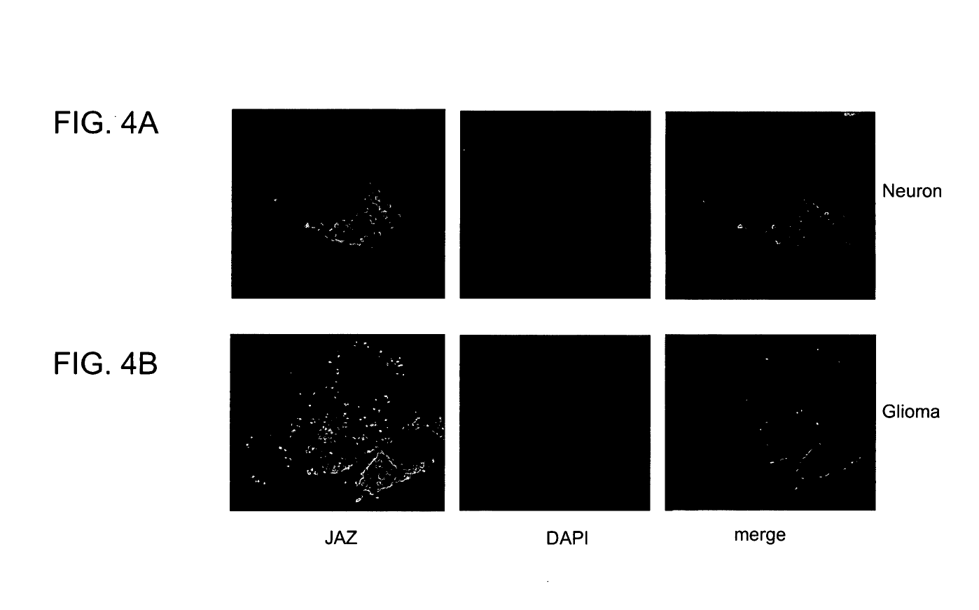

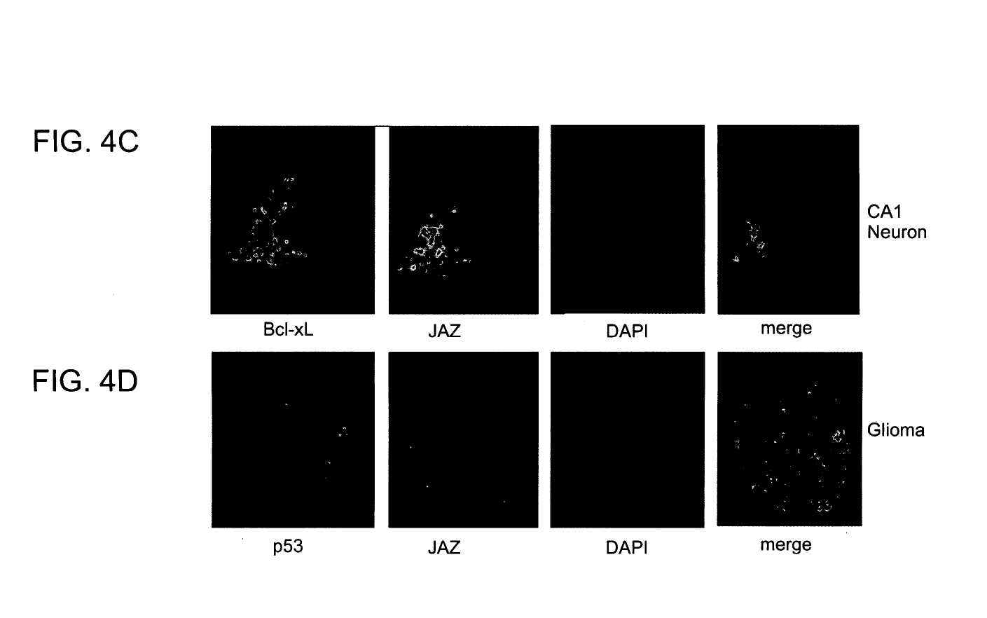

Stress signals, such as serum starvation, leads to increased JAZ expression and subsequently mediate G1 cell cycle arrest and apoptosis likely by regulating p53 transcriptional activity (Yang et al., Blood 108:4136-4145, 2006; FIG. 3A). Hypoxia/ischemia has been suggested to contribute to the formation of pseudopalisading necrosis seen in glioblastoma multiforme and tumor invasion/aggressiveness (FIG. 3B; Brat et al., Lab Invest 2004:84: 397-405, Brat et al., Cancer Res 2004:64: 920-927; Brat et al., Ann Intern Med 2003:138: 659-668). Immunofluorescence analysis indicated that JAZ has unique cytoplasmic expression in the human CNS (FIGS. 4 A, 4B, and 4C), and it was co-expressed with p53 in glioma cells, but in separate sub-cellular compartments (FIG. 4D).

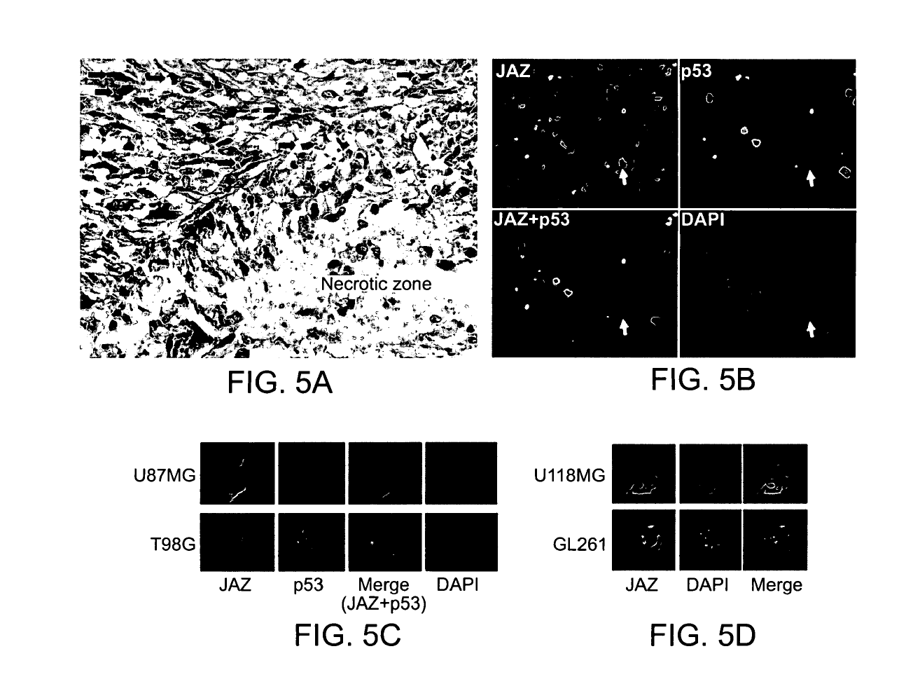

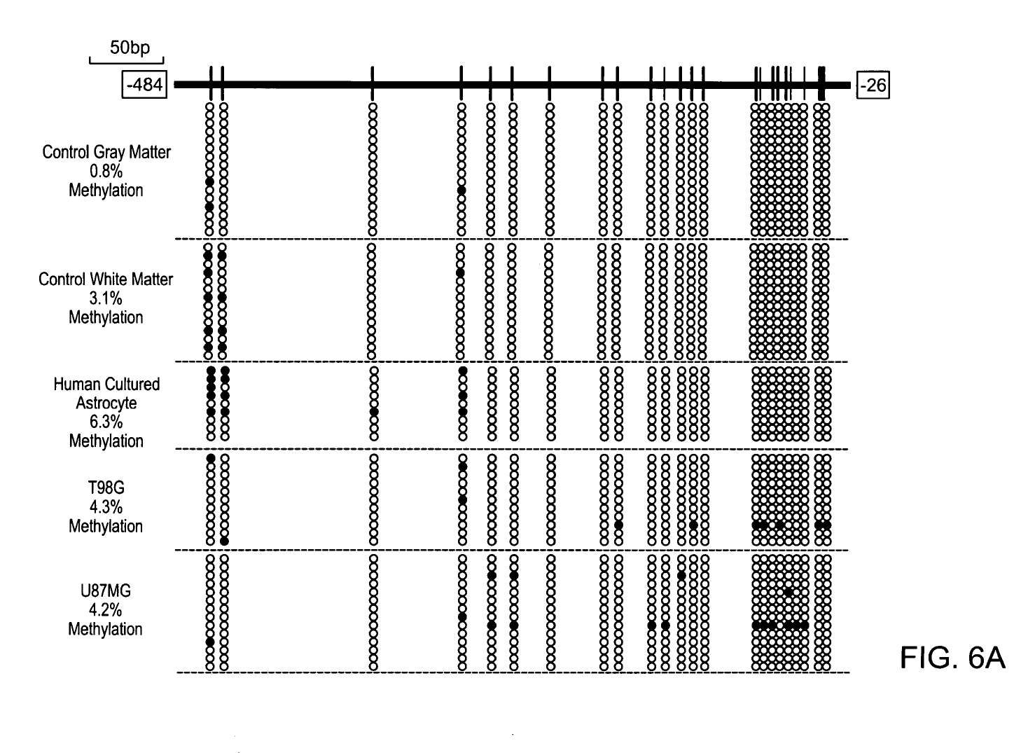

Although the predominant JAZ expression pattern in human CNS is cytoplasmic (FIGS. 1A and 1B), immunohistochemical analysis revealed JAZ nuclear reactivity present in some tumor cells, especially pseudopalisading tumor cells adjacent to areas of necrosis (FIG. 5A), which are presumably under hypoxic stress. Double immunofluorescent analysis for JAZ and p53 showed that increased nuclear JAZ expression in such areas did not co-localize with nuclear p53 expression (FIG. 5B). Immunofluorescence showed that JAZ was present in both the cell nucleus and the cytoplasm in U87MG and T98G glioma cells (FIG. 5C). A lack of nuclear p53 expression in U87MG cells was evident (FIG. 5C) despite strong nuclear JAZ expression. High nuclear p53 expression was detected in T98G cells (FIG. 5C). Immunofluorescence also showed that JAZ was present in both the cell nucleus and the cytoplasm in U118MG and GL261 glioma cells (FIG. 5D).

### Example 3

### No JAZ Gene Mutations were Identified in Glioma Tissues and Glioma Cell Lines

The p53 mutation occurs in less than 30% of primary glioblastomas (Ohgaki et al., Cancer Res 2004; 64:6892-9). Since increased JAZ expression in glioma correlated with increased p53 expression (Table 1), increases in p53 expression could be secondary to increases in JAZ expression. Previous mutational analysis of JAZ showed that the mutations in the zinc finger domains could lead to the loss of JAZ's nuclear/nucleolar localization (Yang et al., J Biol Chem 1999; 274:27399-406). Since JAZ has a predominantly cytoplasmic expression in high grade glioma (FIGS. 2 and 3), it is possible that JAZ is mutated in glioma cells and, therefore, unable to activate p53 cell signaling to promote cell death. All seven JAZ exons from 10 human glioma samples (6 grade IV GBM and 4 grade III anaplastic astrocytoma) and 9 glioma cell lines (U87MG, T98G, U118MG, A172, LN18, DBTRG-05MG, LN229, M059J, WT4) were amplified by PCR and subjected to DNA sequence analysis. No mutations were identified in any of the samples examined (Table 2).

TABLE 2

| | Mutations detected |
|---|---|
| Glioma cell lines (A172, T98, U87MG, | none |
| U118MG, LN18, DBTRG-05MG, LN229, | |
| M059J, WT4) | |
| Glioma samples (10 patients) | none |

Table 2 shows the results of sequencing JAZ gene coding regions in glioma cell lines and human glioma samples (six glioblastoma (WHO grade IV); two anaplastic astrocytoma (WHO grade III); two diffuse astrocytoma (WHO grade II)). No mutations were found in any of the samples examined. These findings indicate that mutations in the JAZ protein coding sequence did not contribute to the increased and predominantly cytoplasmic JAZ expression in high grade glioma.

### Example 4

### DNA Hyper-Methylation or Gene Mutations were Not Associated with Increased JAZ Expression

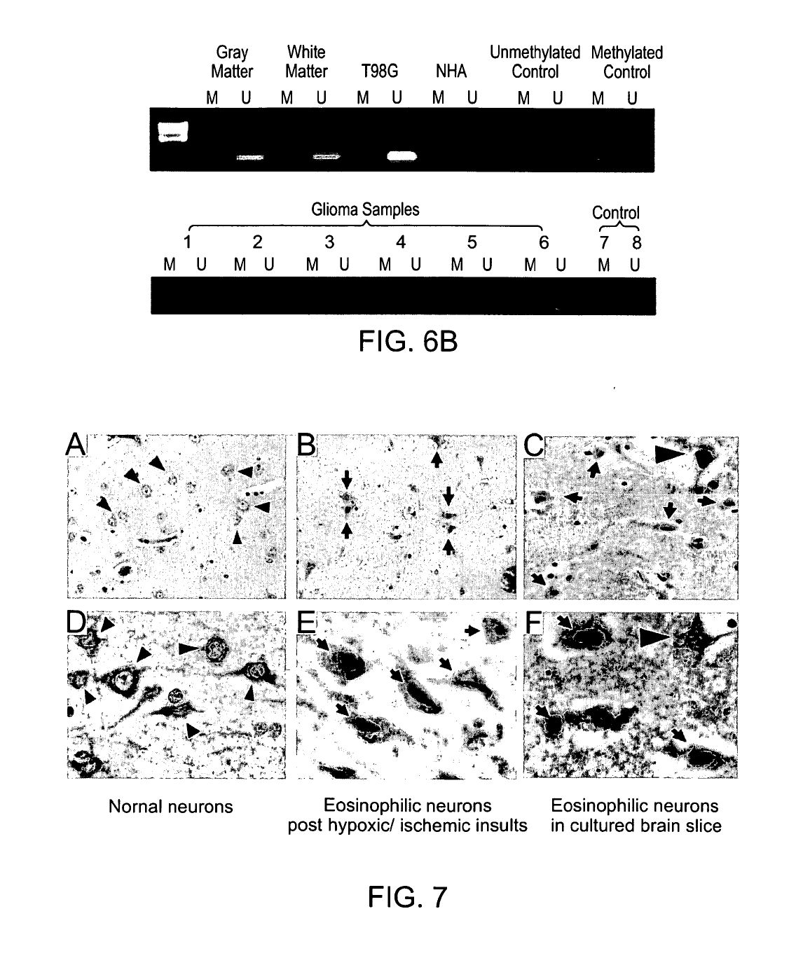

Gliomas, like other tumor types, arise from a complex and poorly understood sequence of genetic and epigenetic alterations. There is evidence that epigenetic alterations leading to gene silencing or expression, in the form of aberrant CpG island promoter hyper- or hypo-methylation, respectively, contribute to glioma development. The JAZ promoter region contains a CpG island upstream of the transcription start site. This prompted the investigation of whether an aberrant DNA methylation pattern might contribute to the significantly increased JAZ expression observed in high grade glioma. Bisulfite genomic sequencing (BGS) showed that there was no significant differences in the methylation status of 23 CpG sites (−26 to −476 relative to the transcription start site) among non-neoplastic control brain tissues (gray matter and white matter), normal cultured human astrocytes, T98G, and U87MG glioma cell lines (FIG. 6A). Most of these CpG sites were un-methylated in the specimens examined. Methylation specific PCR (MSP) analysis showed that the MSP products in control brain tissues, normal cultured human astrocytes, T98G glioma cell lines, and 6 human GBM samples were all unmethylated (FIG. 6B). Thus, DNA methylation is not involved in the increased JAZ expression seen in high grade gliomas.

### Example 5

### Increased Cytoplasmic to Nuclear JAZ Re-Distribution was Observed in Hypoxic/Ischemic Damaged Neurons

Human CNS neurons undergo cell death in response to hypoxia/ischemia. Immunohistochemical studies showed that JAZ was redistributed from the cytoplasm into the nucleus in hypoxic-ischemic cerebral cortical neurons (FIGS. 7A and 7B, 7D and 7E). Nuclear JAZ was also identified in hypoxic/ischemic hippocampal CA1 neurons and cerebellar Purkinje neurons, neurons that are especially sensitive to hypoxia. The brain slice culture model is a well known experimental system for creating hypoxic/ischemic injury to neurons (Longo et al., Reprod Fertil Dev 1995; 7:385-9; Kohling et al., Brain Res 1996; 741:174-9; Toner et al., Brain Res 2002; 958:390-8). Immunohistochemical analysis of JAZ in brain slice tissue showed that while normal neurons maintained their cytoplasmic JAZ expression, neurons showing ischemic cell changes (eosinophilic neurons) exhibited strong nuclear JAZ reactivity (FIGS. 7C and 7F).

### Example 6

### Reduced JAZ Expression Lead to Reductions in Glioma Cell Viability, which was Independent of p53 Expression Status

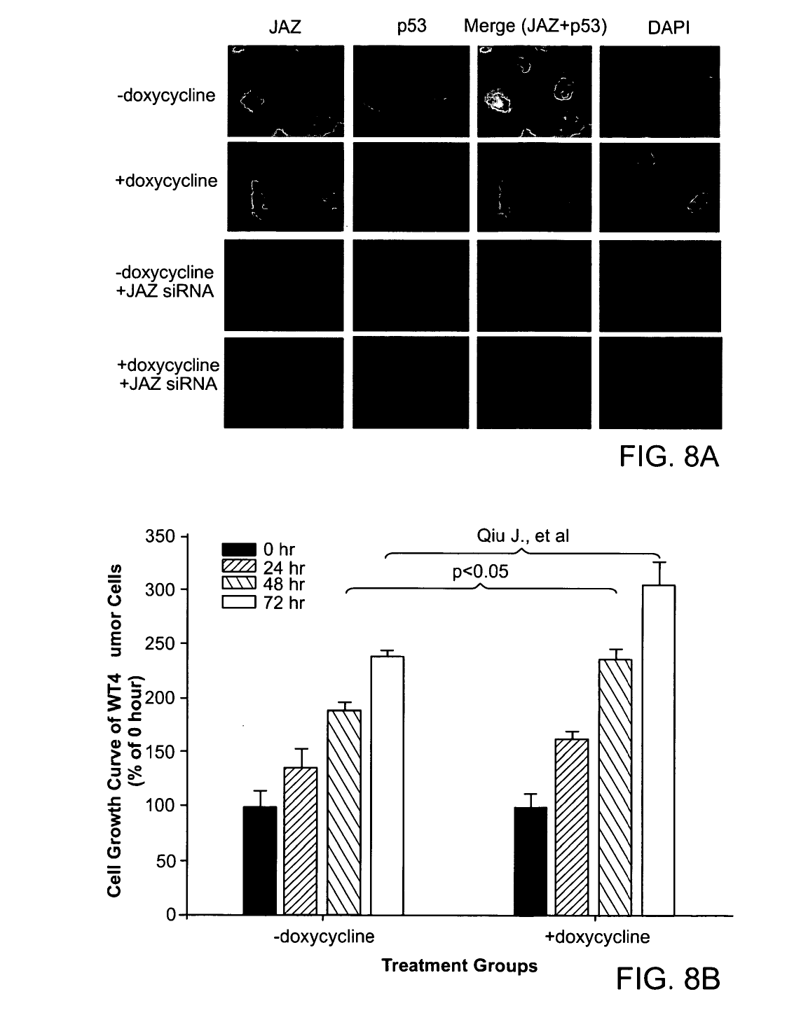

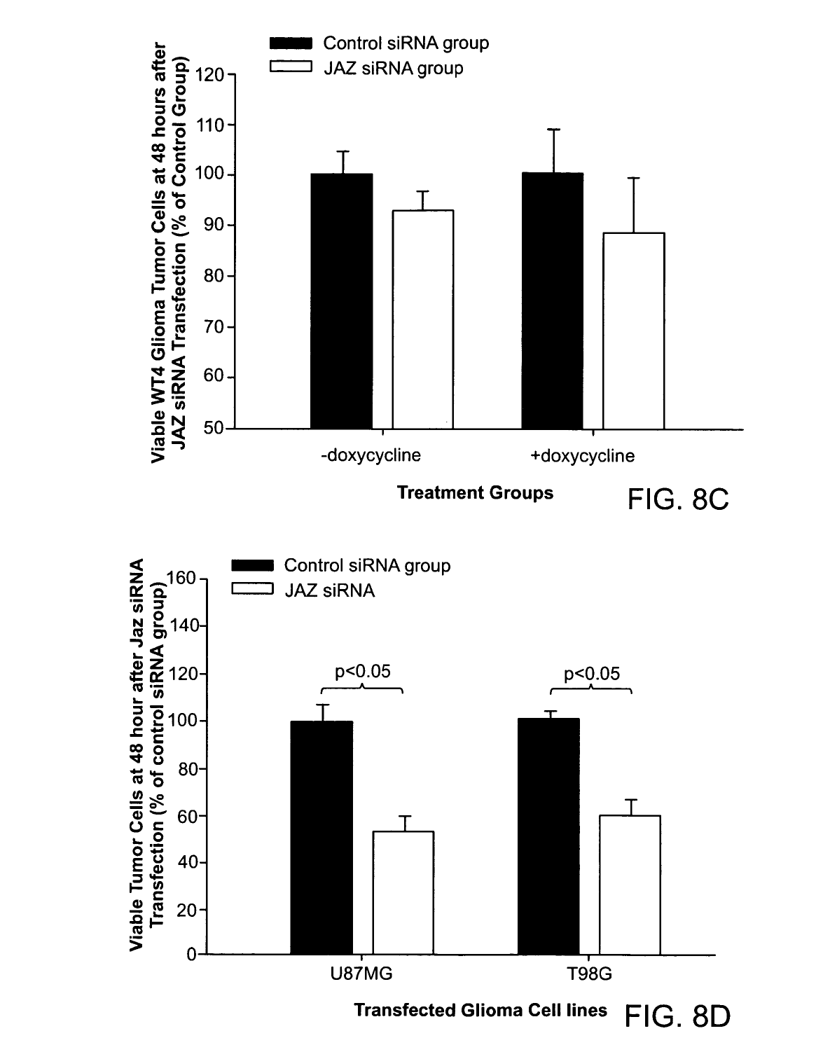

Since JAZ is highly expressed in high grade glioma, the effect of JAZ knockdown on glioma cells was analysed using transfection of JAZ siRNA. WT4 glioma cells contain wild type p53. The cell lines contain a p53 encoding sequence that is under the control of a tetracycline-sensitive promoter. p53 is produced in the absence of the tetracycline/doxycycline, but not in the presence of tetracycline/doxycycline. Cells were co-stained for p53 and JAZ. Double immunofluorescence showed that JAZ labeling was present in both the cell nucleus and the cytoplasm of WT4 glioma cells (FIG. 8A). There was strong nuclear p53 expression in WT4 cells without the presence of doxycycline and p53 expression was completely inhibited with the presence of doxycycline (2 μg/ml) (FIG. 8A). Cell viability assays showed that the non-p53-expressing WT4 cells exhibited more rapid growth when compared with p53-expressing WT4 cells (FIG. 8B). There was a mild decrease in the number of viable tumor cells for both p53-expressing and p53-non-expressing WT4 cells after transfection with JAZ siRNA, although the decreases were not statistically significant (FIG. 8C). Cell viability assays showed that cell growth was significantly decreased in both U87MG and T98G glioma cell lines after JAZ siRNA transfection (FIG. 8D). Forty-eight hours after transfection, there was a 46.4% decrease in viability of U87MG glioma cells and a 39.5% decrease in viability of T98G glioma cells when compared with control siRNA transfected groups (p<0.05). These results indicate that JAZ is likely to be involved in promoting glioma tumor cell survival that is independent of p53 expression status. In contrast to previous reports indicating that JAZ is a nuclear/nucleolar protein, the results reported herein indicate that JAZ is predominantly cytoplasmic in malignant gliomas and in CNS neurons. A significant increase in JAZ expression was observed in malignant gliomas when compared with that present in normal glia. Very interestingly, increased nuclear JAZ expression was observed in pseudopalisading tumor cells of human glioblastoma. JAZ has both cytoplasmic and strong nuclear expression in T98G, U87MG, U118MG, WT4 and GL261 glioma cell lines. P53 mutations occur in less than one third of primary glioblastoma (Ohgaki et al. Cancer Res 2004; 64:6892-9), and prior mutational analyses showed that zinc finger domain mutations led to the loss of JAZ's nuclear/nucleolar localization (Yang et al., J Biol Chem 1999; 274:27399-406). Because all 13 glioblastoma cases in the glioma tissue microarray were primary glioblastoma cases, it is possible that mutations in JAZ lead to the cytoplasmic JAZ localization and/or the inability of nuclear JAZ to activate wide-type p53 cell death signaling pathway in glioma cells. Surprisingly, JAZ coding region mutations were not observed in the ten human glioma samples (six glioblastoma (WHO grade IV); two anaplastic astrocytoma (WHO grade III); two diffuse astrocytoma (WHO grade II) and nine glioma cell lines (include A172, T98, U87MG, U118MG, LN18, DBTRG-05MG, LN229, M059J, WT4) that were examined. Therefore, DNA mutations did not seem to have contributed to the increased and predominantly cytoplasmic JAZ expression in glioma cells and the inability of activation of JAZ-p53 cell death signaling pathway. The increased p53 expression observed in these glioblastomas may be unrelated to the increased JAZ expression.

Although increased nuclear JAZ expression was observed in pseudopalisading tumor cells in addition to strong cytoplasmic JAZ expression, the nuclear and cytoplasmic JAZ expression did not correlate with increased nuclear p53 expression in these tumor cells. To address the role of increased JAZ in glioma tumor cells and JAZ's interaction with p53 in glioma cells, JAZ' function was characterized using tet-off controlled p53 expression WT4 glioma cell line. The WT4 tumor cells contained a p53 encoding sequence under the control of a tetracycline/doxycycline sensitive promoter (Van Meir et al., Nat Genet 1994; 8:171-6); transcription of wild-type p53 in these cells was suppressed in the presence of tetracycline/doxycycline. JAZ was highly expressed in WT4 glioma cells with both nuclear and cytoplasmic patterns regardless of p53 expression status. P53 expression could be completely inhibited in the presence of doxycycline. The non-p53-expressing WT4 cells grew slightly faster than the p53-expressing WT4 cells, which is consistent with the role of wild-type p53 as a tumor suppressor gene. The interaction of JAZ with p53 in p53-expressing WT4 cells likely contributed to the delayed cell growth (Yang et al., Blood 108: 4136-4145, 2006). Therefore, knocking down JAZ expression in p53-expressing WT4 cells was expected to result in an increase in viable tumor cells. Surprisingly, forty-eight hours after knocking down of JAZ expression by JAZ siRNA transfection, a decrease in both p53-expressing and p53 non-expressing tumor cells was observed when compared with that of the control siRNA treated group. Wild-type p53 expressing U87MG and mutant p53 expressing T98G glioma cells were used in further JAZ siRNA transfection experiments to confirm that the observations were not associated with the transfection agent. In these studies, a significant reduction in viable tumor cells was observed in both U87MG and T98G glioma cells at forty-eight hours after JAZ siRNA transfection. These results indicate that JAZ is very likely involved in promoting glioma tumor cell survival that is independent of p53 expression status. The JAZ siRNA knockdown results reported herein were unexpected because they contradicted previous reports that JAZ siRNA knock down delayed cell death in NFS/N1.H7 murine myeloid and M1 murine myeloid leukemic cells (Yang et al., Blood 108: 4136-4145, 2006). This difference could be explained by JAZ possessing cell-type and species-specific functions: JAZ has strong cytoplasmic expression in addition to nuclear JAZ expression in T98G, U87MG, and WT4 glioma cell lines and may have p53-independent functions, whereas in both the mouse NFS/N1.H7 and M1 cell lines, JAZ was predominantly expressed in the nucleus and functions through its regulation of p53 transcription activity.

Hypoxia/ischemia is known to play an important role in glioma development, particularly in glioblastoma. Serum deprivation has been shown to trigger increased JAZ expression in vitro (Yang et al., Blood 108: 4136-4145, 2006). An up-regulation of JAZ in high grade gliomas could be the result of tumor cells responding to hypoxic/ischemic stress. Pseudopalisading glioma cells that are concentrated around necrotic zones in glioblastoma are believed to be under hypoxic stress, have increased expression of hypoxia-inducible factor 1-alpha (HIF-1alpha), and are mostly composed of actively migrating tumor cells. Since the glioma cell lines (U87MG, T98G, U118, and GL261) examined also showed similar strong nuclear and cytoplasmic JAZ expression, it is likely that these commercially available glioma cell lines may either have been derived from nuclear JAZ expressing pseudopalisading tumor cells, which represent an aggressive population of glioma cells, or were transformed from other predominantly cytoplasmic JAZ expressing tumor cells in the original tumors. Since JAZ expression is increased in response to serum deprivation, it is possible that the JAZ promoter may contain HIF-1alpha binding sites that are responsible for increased JAZ expression under hypoxic/ischemic stress. However, examination of the transcription factor binding sites within the JAZ promoter did not identify a HIF-1alpha binding site, nor were there binding sites for other well known hypoxia response transcription factors, e.g. c-myc, c-fos, AP-1.

It is well documented that epigenetic alterations can lead to either aberrant gene silencing or elevated expression, which in turn may cause early transformation, cancer progression, or increased resistance to therapy. DNA methylation, in the form of aberrant CpG island promoter hyper- or hypo-methylation, is one of the most common epigenetic changes and has been shown to contribute to gliomagenesis. It has also been reported that DNA hypomethylation can occur under hypoxic conditions in breast cancer, colorectal cancer, and melanoma. Hypoxia can cause DNA hypomethylation in p161NK4a and IGF2 promoters in breast and colorectal cancers. While studying the JAZ promoter for hypoxia related transcription factor binding sites, a CpG island spanning the transcription start site was identified. To determine whether JAZ expression was turned on in glioma cells by hypomethylation of its promoter, the CpG site DNA methylation status was examined using both BGS and MSP techniques. No significant differences in DNA methylation were found among control human brain tissues, high grade glioma tissues, normal cultured human astrocytes, and the tested glioma cell lines. These results suggest that DNA hypomethylation of JAZ promoter CpG sites is unlikely to contribute to increased JAZ expression in glioma. The role of other hypoxia induced JAZ gene expression regulators including epigenetic changes like histone acetylation warrants further investigation.

Another very interesting observation was the differential JAZ expression patterns between normal neurons and hypoxic/ischemic injured neurons. Normal human cortical neurons showed almost exclusive cytoplasmic JAZ. But after hypoxic/ischemic injury, neurons undergoing ischemic cell changes (“eosinophilic” neurons) showed a strong increase in nuclear JAZ immunoreactivty with a significant decrease of cytoplasmic JAZ. These findings were confirmed in a brain slice culture model that is widely used to study neuronal hypoxic/ischemic injury (Longo et al., Reprod Fertil Dev 1995; 7:385-9; Kohling et al., Brain Res 1996; 741:174-9; Toner et al., Brain Res 2002; 958:390-8). It has been previously reported that JAZ can act as a cargo protein in the exportin-5 nuclear transport system (Chen et al., Mol Cell Biol 2004; 24:6608-19). Exportin-5 is one of the karyopherin proteins that can mediate the nuclear export of tRNA, pre-miRNAs, and shRNAs and plays an important role in miRNA biogenesis and function. It has been well documented that miRNA plays important roles in cancer development, including gliomagenesis. It would be very interesting to know whether JAZ is involved in glioma development through its potential role in facilitating miRNA transport. This latter role could be one of JAZ's p53-independent functions in the human CNS. Alternatively, our double IF analysis showed that JAZ and Bcl-xL co-localized in the cytoplasm of human hippocampal neurons with a granular pattern (data not shown). Such co-localization was also observed in cerebral cortical neurons. Since Bcl-xL is mainly a mitochondrial protein involved in cell survival, the co-localization of JAZ with Bcl-xL suggests JAZ may have some functions in the mitochondria, another possible p53-independent function in human CNS.

In summary, JAZ was found to have significantly increased expression in high grade gliomas with a novel, predominantly cytoplasmic expression pattern. Up-regulation of JAZ, including increased nuclear JAZ, could be due to hypoxic/ischemic stress and may contribute to glioma cell growth. JAZ is likely to function in p53-independent pathways in the gliomas as well as in the human CNS. Reduction of JAZ expression can lead to the reduction of the glioma cell growth, suggesting that JAZ might be a potential novel target for glioma treatment.

### Example 7

### J1 Reduced Glioma Cell Viability

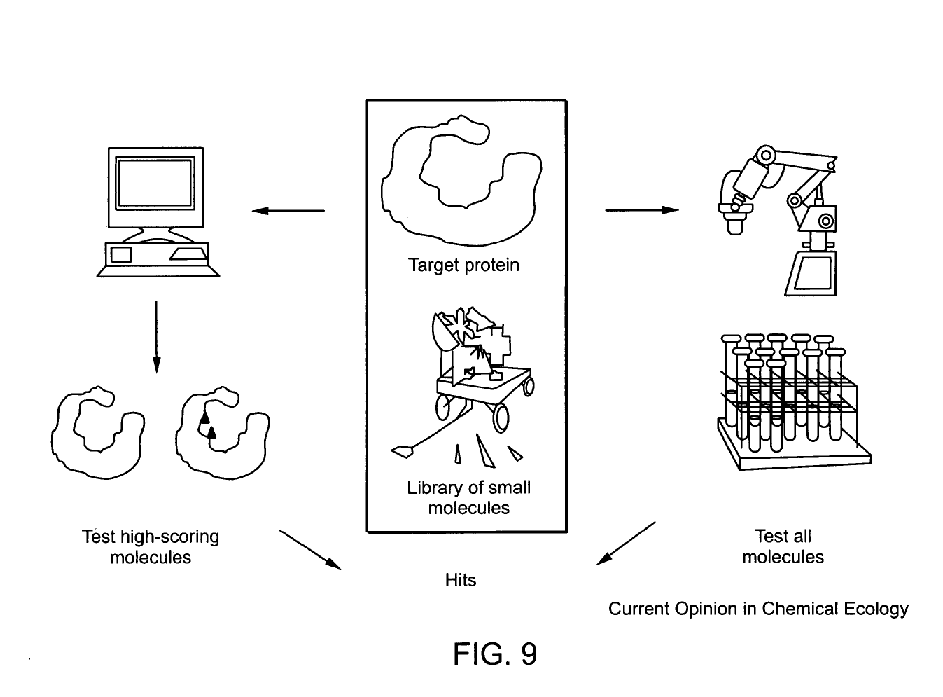

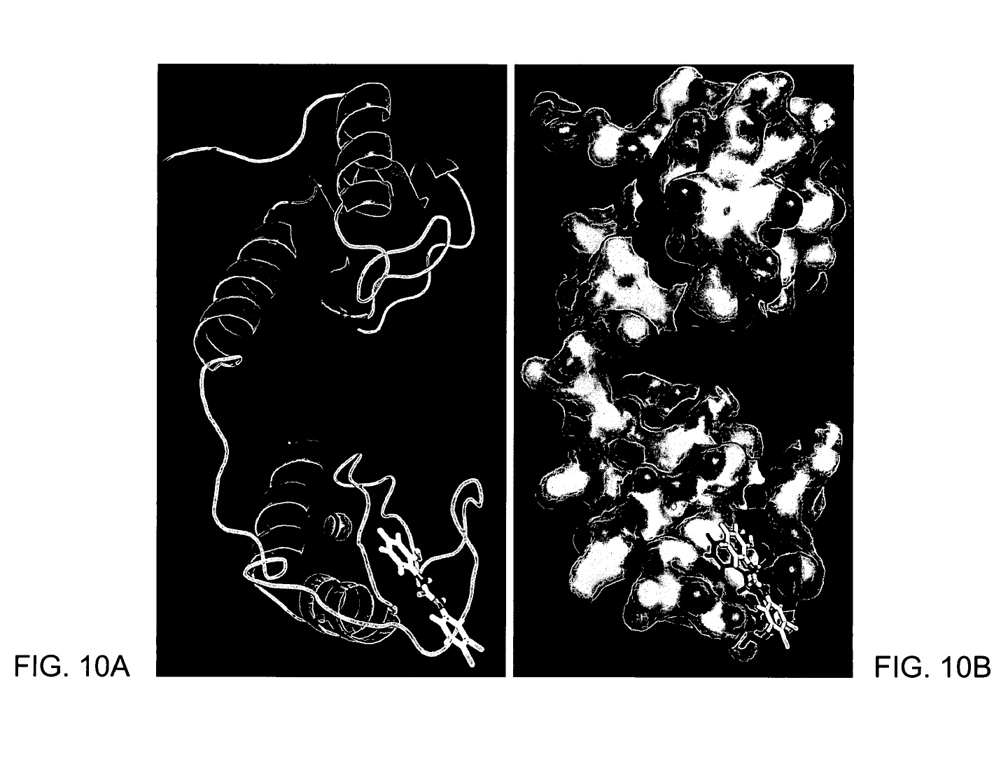

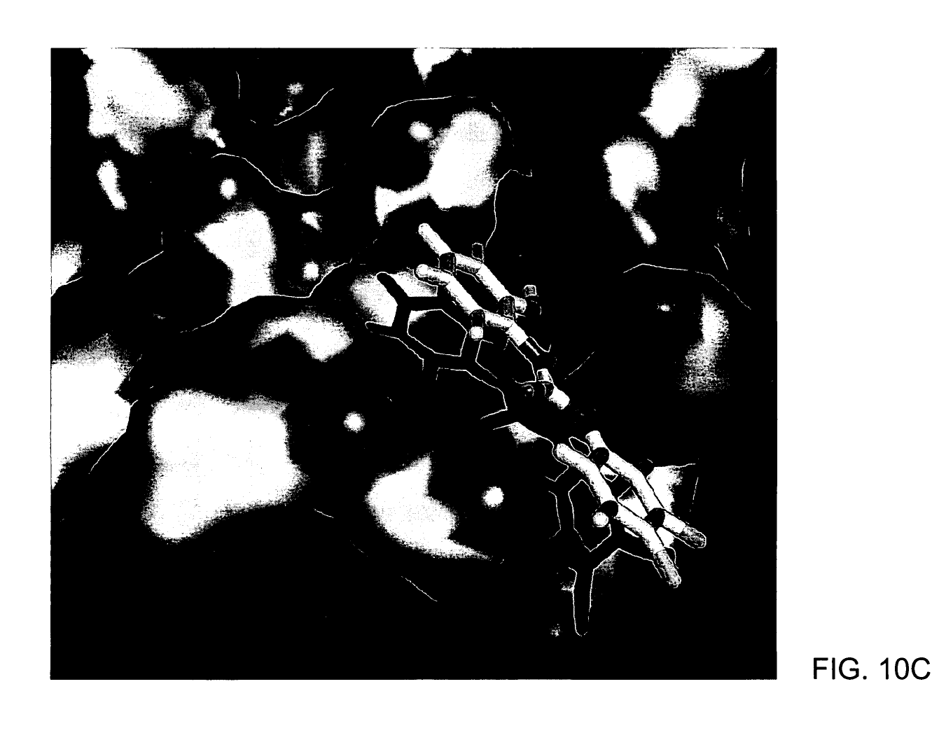

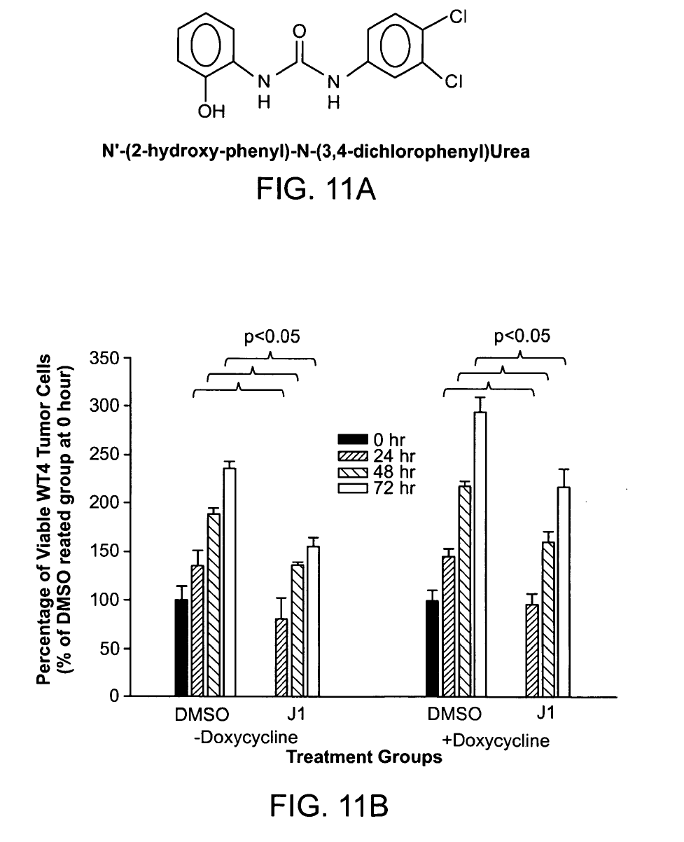

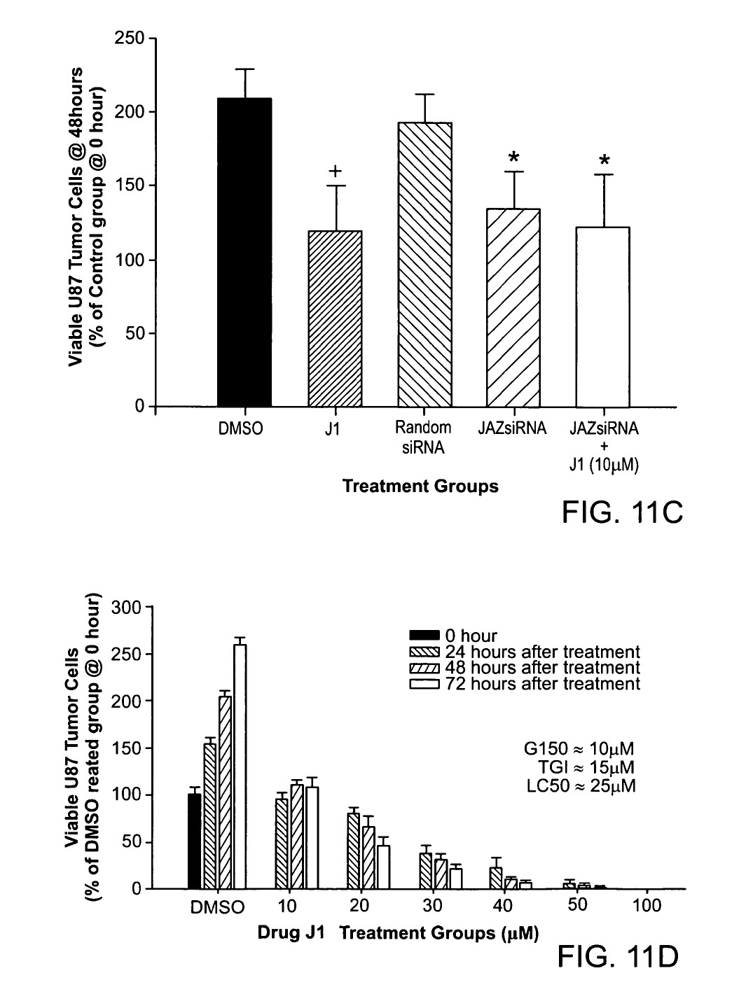

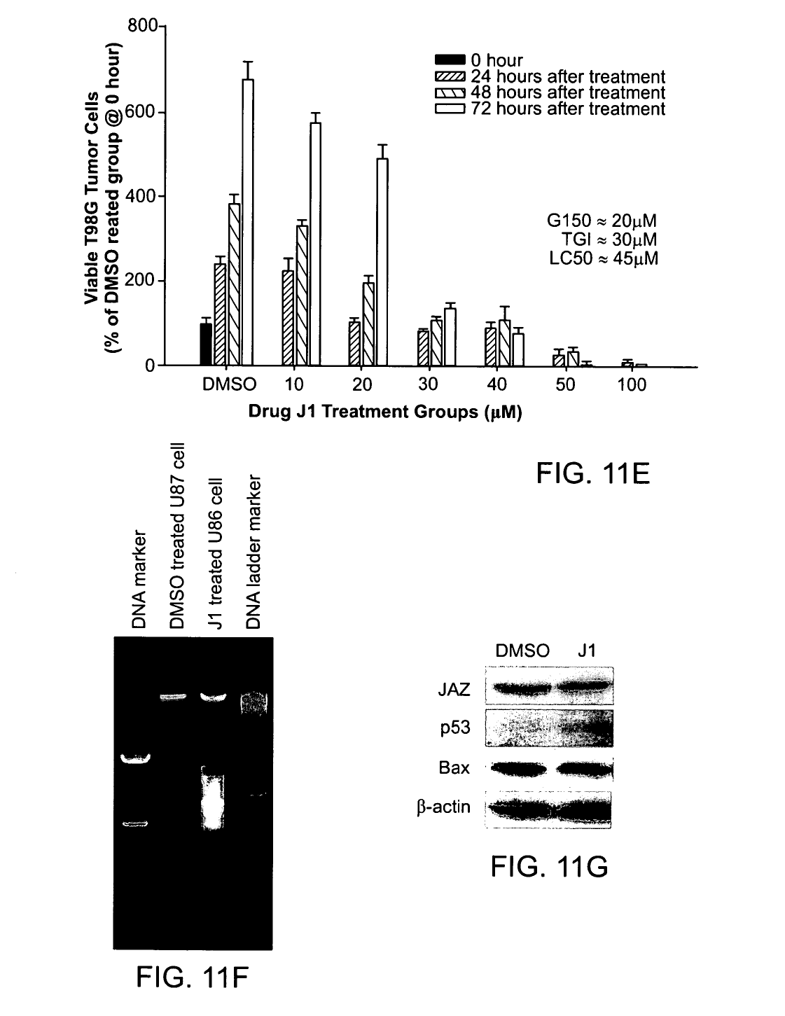

Relative to other zinc finger proteins, JAZ has longer linker sequence between its zinc finger domains (Yang et al., J Biol Chem 1999:274: 27399-27406Chen et al., Mol Cell Biol 2004:24: 6608-6619). Interestingly, a JAZ structural binding pocket was identified by generating an atomic homology model. Molecular docking, which is a structure-based drug design approach, provided for the identification of compounds that block JAZ's function and reduce glioma growth. This strategy was used to screen approximately 300,000 small drug-like molecules for their ability to interact with a structural pocket unique to JAZ. A molecular docking strategy used to identify such compounds that is shown in (FIG. 9). Molecular docking models showing the position of the JAZ inhibitor J1 in the JAZ structural binding pocket is shown at FIGS. 10A, 10B, and 10C. Twelve JAZ-targeting molecules were identified using such methods and obtained from the NCI developmental therapeutics program repository. Exemplary compound J1 (FIG. 11A) showed significant cytotoxic effects on glioma cell lines (FIGS. 11B-11E). Increased DNA laddering was observed in U87MG glioma cells following J1 treatment (25 μM) (FIG. 11F). J1 treatment (25 μM) did not alter JAZ, p53, and Bax protein levels (FIG. 11G).

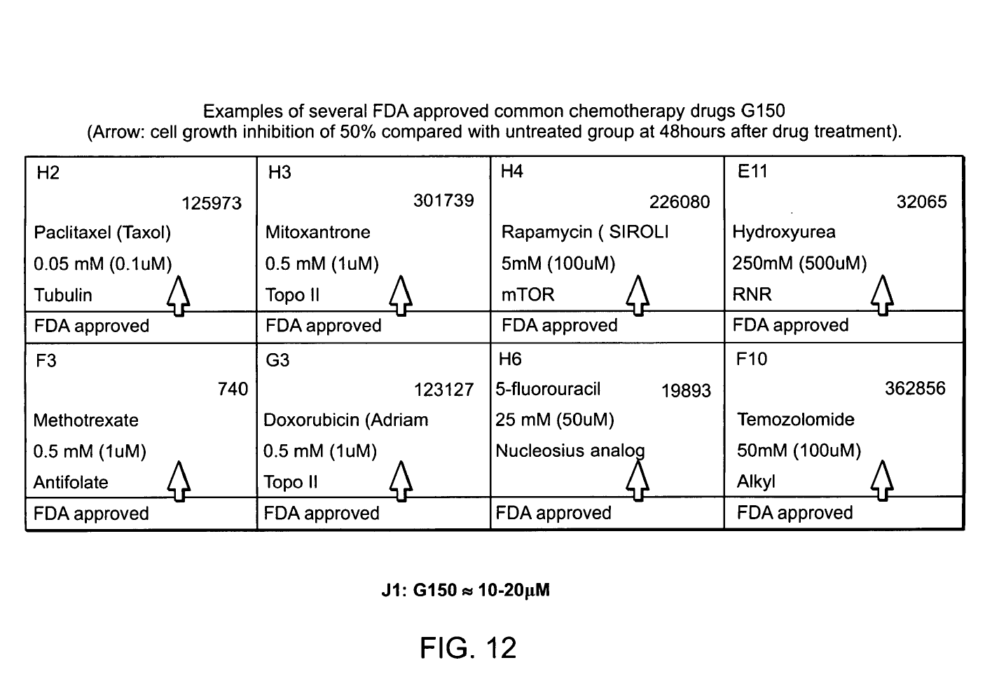

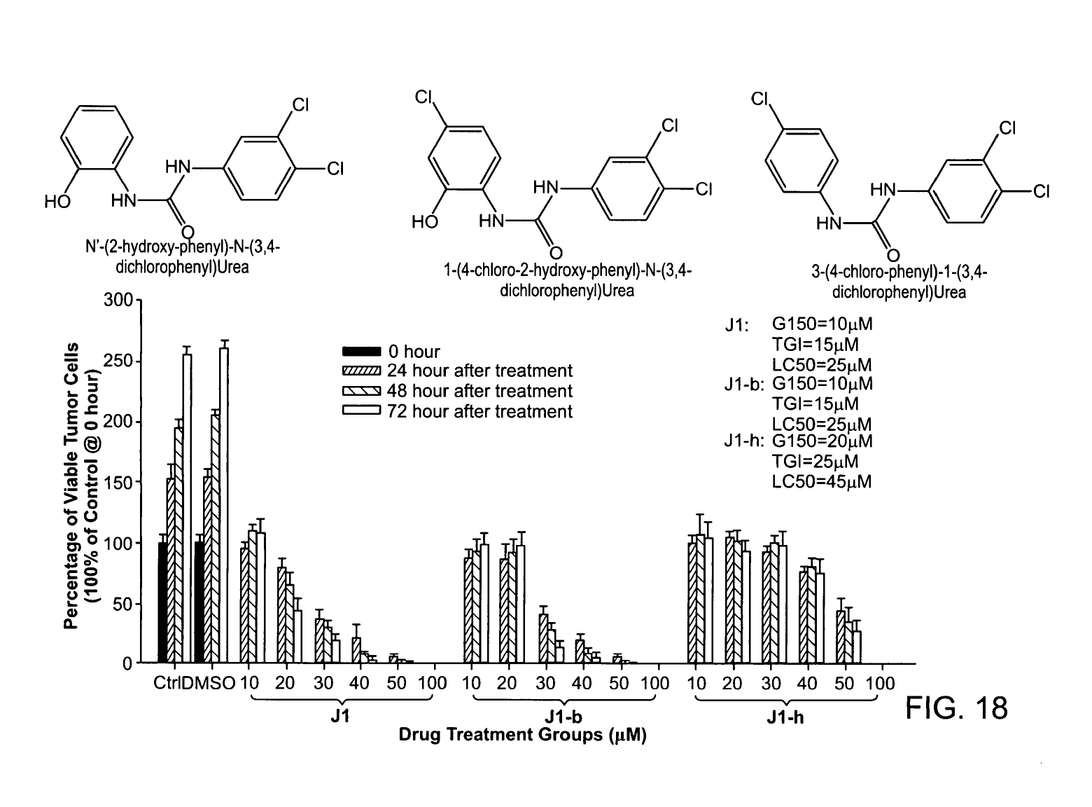

J1 significantly reduced glioma cell viability. This effect was much more potent than that of TMZ (FIG. 12). J1 includes two phenyl rings, two chloro groups (—Cl) and one hydroxyl group (—OH) present in J1 chemical structure (FIG. 18). The positions of the two choloro groups and one hydroxyl group on the two phenyl rings likely function in J1's anti-cancer effect (FIG. 16). Synthetic derivatives of J1 have been obtained. These derivatives have alterations in the locations of chloro groups and hydroxyl group on the phenyl rings. Preferred J1 derivatives are selected by assaying for glioma cytotoxic effects as described above. Preferred J1 derivatives show increased glioma cytotoxic effect and have an enhanced synergistic effect when administered in combination with TMZ. Selected compounds also have reduced side effects and intolerance.

Other compounds identified by molecular docking studies are shown in Table 3.

TABLE 3
Compounds for Neoplasia Inhibition

| Compound | |
|---|---|
| Name/Chemical | |
| Formula/CAS Number | Structure |
| N-(3-chlorophenyl)- N′-(2-hydroxy- phenyl)Urea | 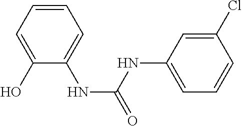 |
| N-(3,4-dichloro- phenyl)-N′-(3- hydroxyphenyl) Urea | 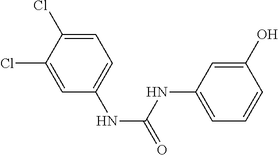 |
| N-(3,4-dichloro- phenyl)-N′-(4- hydroxyphenyl) Urea | 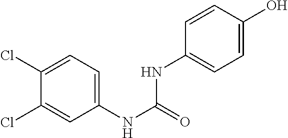 |
| 1-(5-chloro-2- hydroxyphenyl)-3- (3,4-dichlorophenyl) urea | 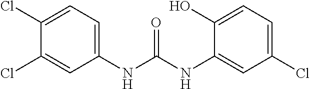 |
| 3,4-Dichloro- phenylurea | 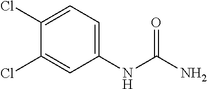 |
| l-(3′,4′-dichloro- phenyl)-3-(4′- chlorophenyl)Urea (Triclocarban) | 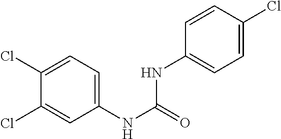 |
| 1-(4-chloro-2- hydroxy- phenyl)-3-(3,4- dichlorophenyl)urea | 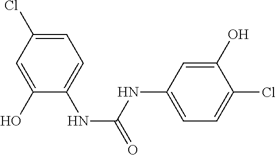 |
| 3-(4-chloro-2- hydroxy- phenyl)-1-(4,5- dichloro- 2-hydroxy- phenyl)urea | 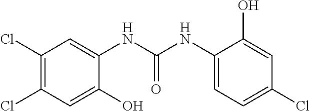 |
| 3-(4-chlorophenyl)- 1-(4,5-dichloro-2- hydroxy-phenyl)urea | 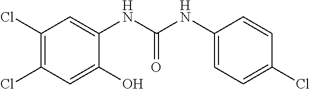 |
| 1-(5-chloro-2- hydroxy- phenyl)-3-(4- chlorophenyl)urea | 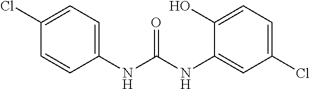 |
| 3-(4-chlorophenyl)- 1-(2-hydroxyphenyl) urea | 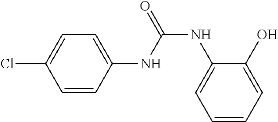 |
| 1-(5-chloro-2- hydroxy-phenyl)-3- phenyl-urea | 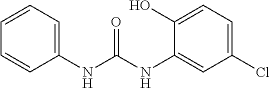 |
| 1-(5-chloro-2- hydroxy- phenyl)-3-(3,5- dichlorophenyl) urea | 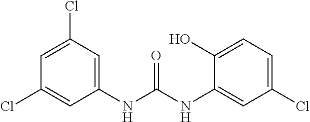 |
| 1-(3,4-dichloro- 2-hydroxy-phenyl)- 3-(2,3-dichloro- phenyl) urea | 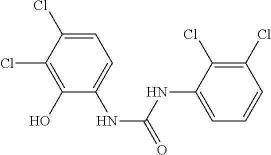 |

### Example 8

### J1 and Analogs thereof are Cytotoxic to Neoplastic Cells

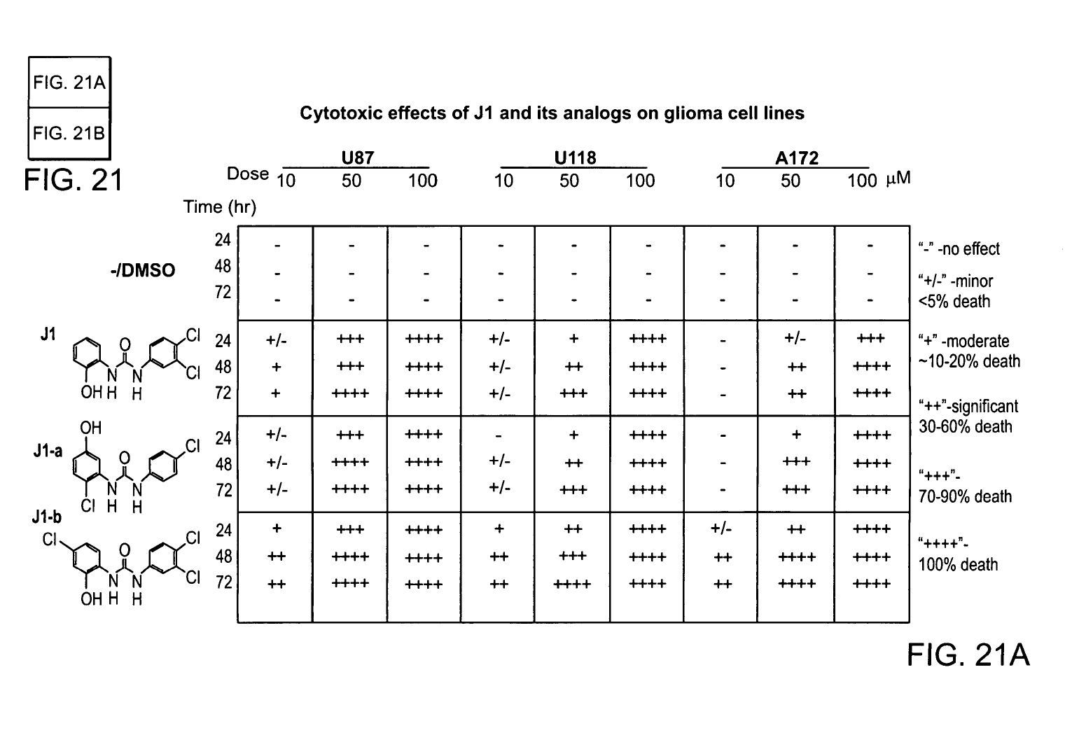

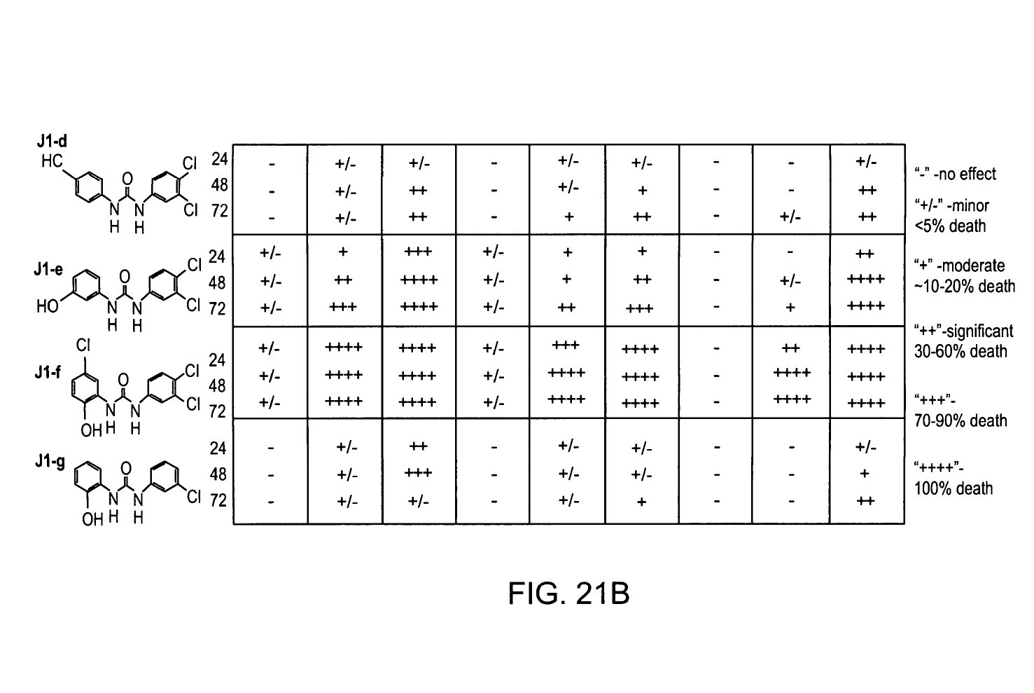

As shown in FIGS. 11A-11E, J1 is cytotoxic to U87 glioma cells. The cytotoxicity of J1 is independent of p53, as shown in FIG. 11B. Six substituted diphenyl urea compounds related to J1 were also shown to be cytotoxic to neoplastic cells (FIG. 21). The presence of a chloral group on the right phenyl ring enhances J1's cytotoxic effect (FIG. 21). The presence of an additional chloral group (arrow) on the left phenyl ring of J1 and the presence and location of a hydroxyl group on the left phenyl ring also affect cytotoxicity(FIG. 21).

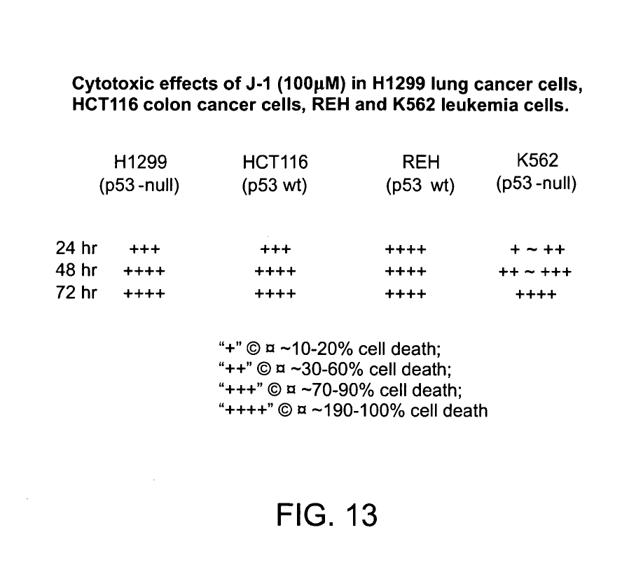

J1 is not only effective for the treatment of glioma. As shown in FIG. 13, J1 is also cytotoxic to lung cancer, colon cancer and leukemia cells.

### Example 9

### J1 Treatment of Glioma Cells Significantly Modulates Cellular Protein Levels

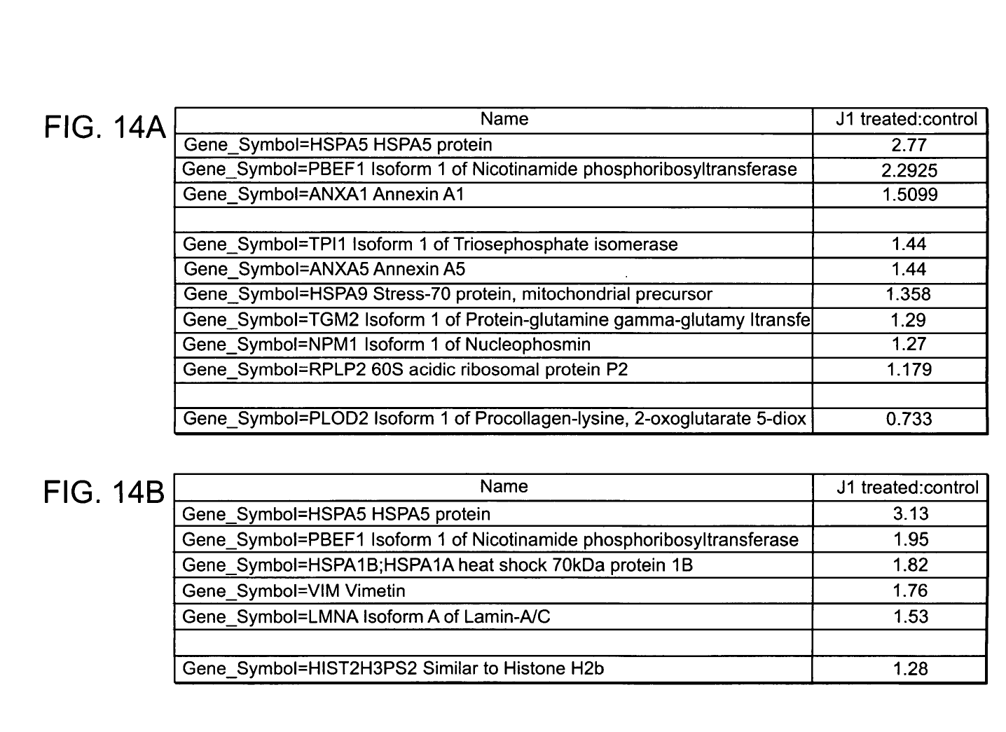

To assess the global effects on protein levels of J1 treatment of glioma cells, quantitative analysis of proteome changes was performed upon glioma cells treated with J1 (FIGS. 14A and 14B). Results of such experiments in U87 glioma cells are shown in FIG. 14A. Results in U118 glioma cells are shown in FIG. 14B

Proteins that were up-regulated in U87 glioma cells by J1 treatment included PBEF1, HSPA5, ANXA1, TPI1, ANXA5, HSPA9, TGM2, NPM1, and RPLP2. Proteins that were up-regulated in U118 glioma cells by J1 treatment included HSPA5, PBEF1, HSPA1B, VIM, LMNA and HIST2H3PS2.

Two proteins were significantly up-regulated by J1 treatment in both U87 and U118 glioma cells. These proteins included HSPA5 and PBEF1.

The HSPA5 protein (Heat Shock Protein A5, GRP78, BIP) has been characterized to act in protein folding and assembly in the endoplasmic reticulum (ER), and is known to play a role in facilitating the assembly of multimeric protein complexes inside the ER. The HSPA5 protein has also been associated with chronic hypoxia in human gastric tumor cells, and has been described as a glucose regulated protein. Expression of HSPA5 protein has been correlated with histologic differentiation and favorable prognosis in neuroblastic tumors.

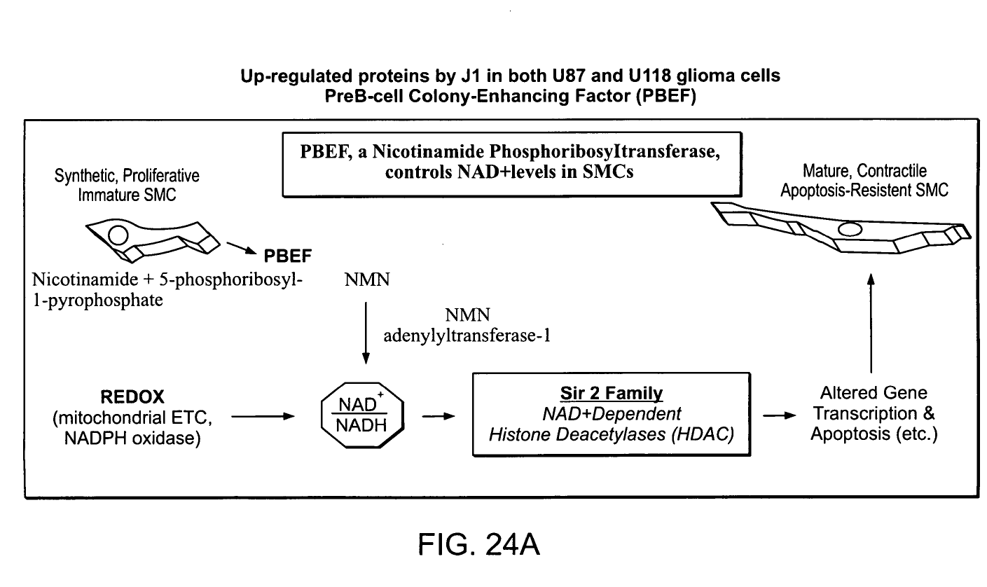

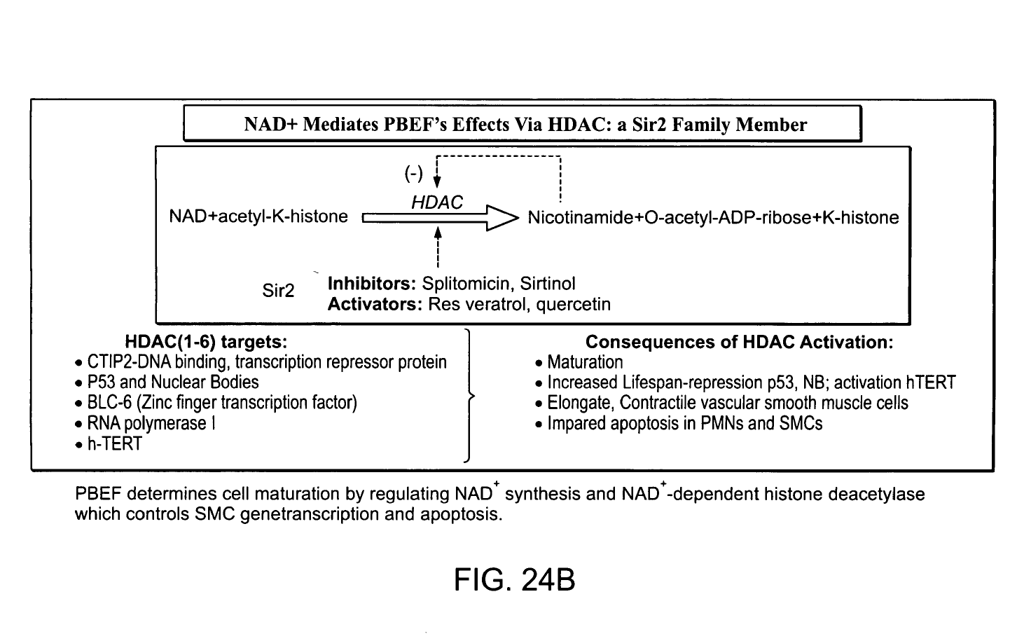

The PBEF protein has been characterized to determine cell maturation by regulating NAD⁺ synthesis and NAD⁺-dependent histone deacetylase which controls SMC gene transcription and apoptosis (FIG. 24).

One protein (PLOD2) was significantly down-regulated in U87 glioma cells by J1 treatment. PLOD2 (Procollagen-lysine, 2-oxoglutarate 5-dioxygenase 2) is a protein previously characterized as a membrane-bound homodimeric enzyme that is localized to the cisternae of the rough endoplasmic reticulum. The PLOD2 enzyme (cofactors iron and ascorbate) has been shown to catalyze the hydroxylation of lysyl residues in collagen-like peptides. The resultant hydroxylysyl groups provide attachment sites for carbohydrates in collagen and, thus, have been described as critical for the stability of intermolecular crosslinks. The PLOD2 protein has also been characterized as induced by hypoxia, and has been described as associated with cancer progression (e.g., breast cancer progression; Chang et al., PLoS Biology, 2004). Expression of the PLOD2 gene has been observed to be increased in the GBM pseudopalisading area (Louis D N, et al., JNEN, 2005). Thus, agents that down-regulate PLOD2 levels can become attractive therapeutic targets.

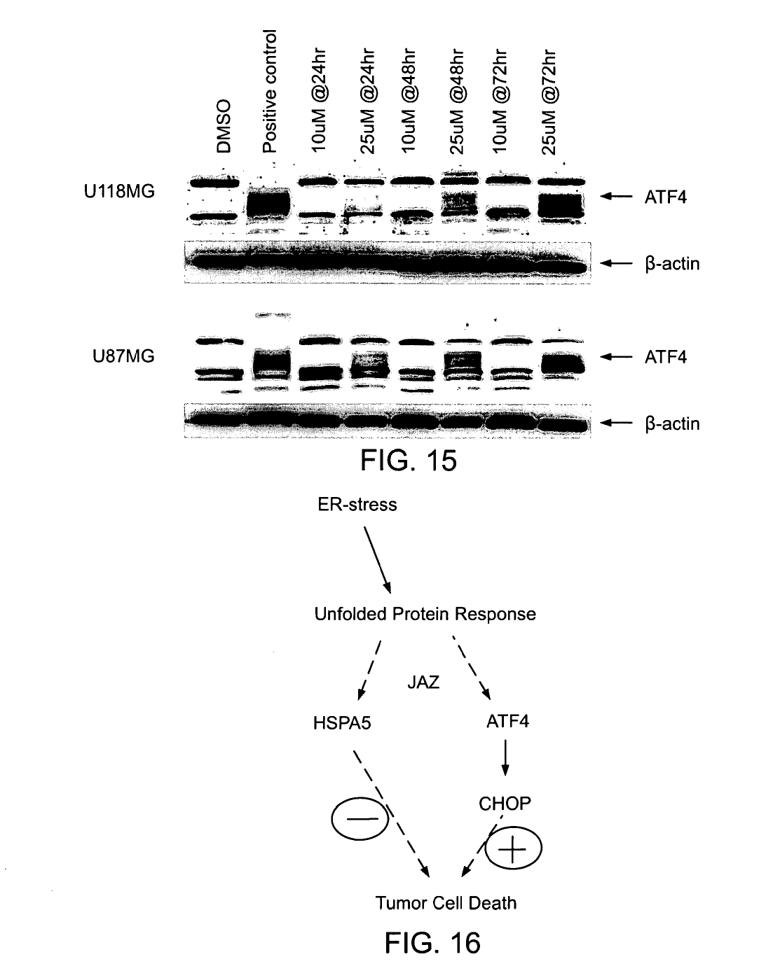

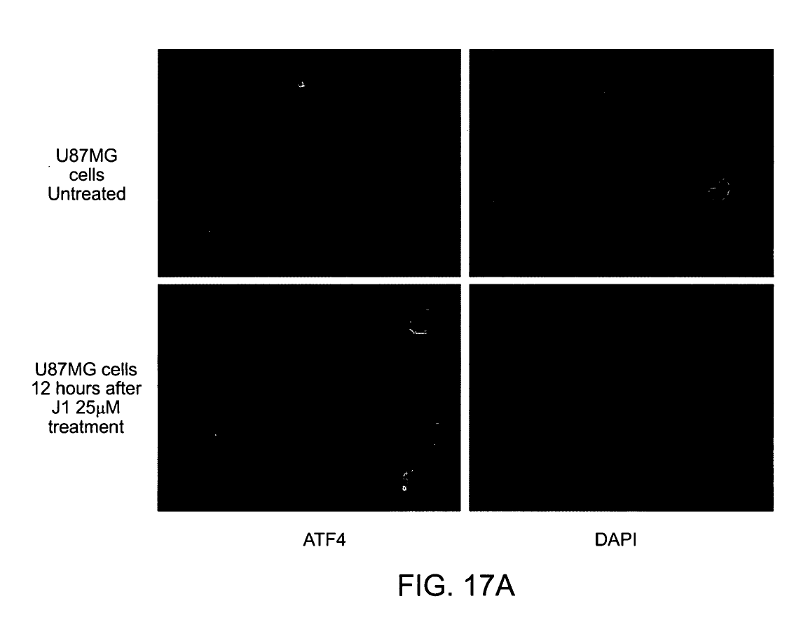

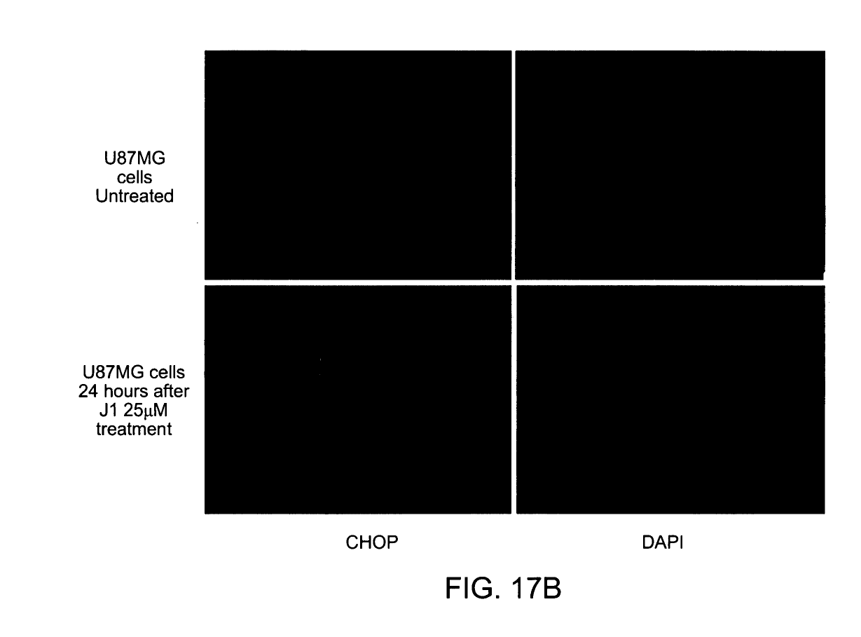

In addition, a time- and dose dependent increase in ATF-4 protein levels was observed at 24, 48, and 72 hours after J1 treatment of U118MG and U87MG cell lines (10 and 25 μM) (FIG. 15) by western blot. ATF-4 is one of the key transcription factors involved in the unfolded protein response pathway, and it is also regarded as a marker for the unfolded protein response pathway (FIG. 16). Changes in CHOP were also observed in U87MG cells after J1 treatment. FIG. 17A shows that nuclear ATF-4 staining was significantly increased in U87 cells at 12 hours after J1 (2504) treatment. FIG. 17B shows that CHOP staining was significantly increased in U87 cells at 24 hours after J1 (2504) treatment.

### Example 10

### J1 Analogues were also Effective in Reducing Tumor Cell Viability

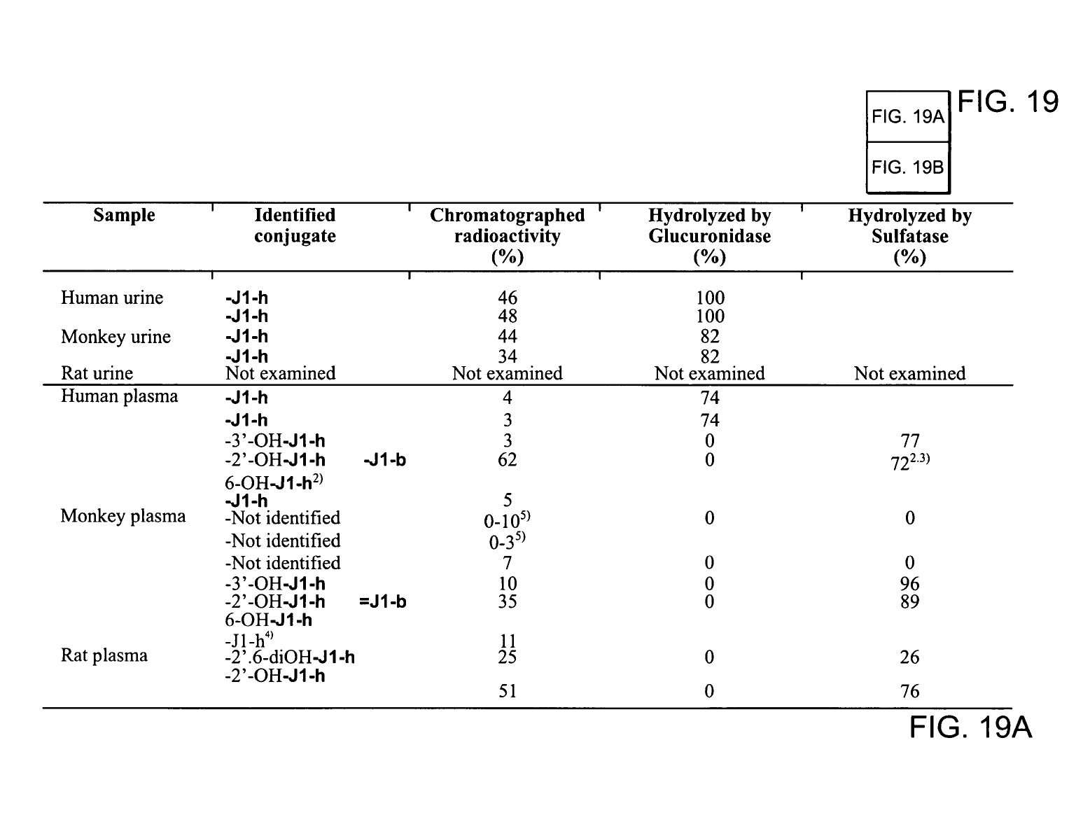

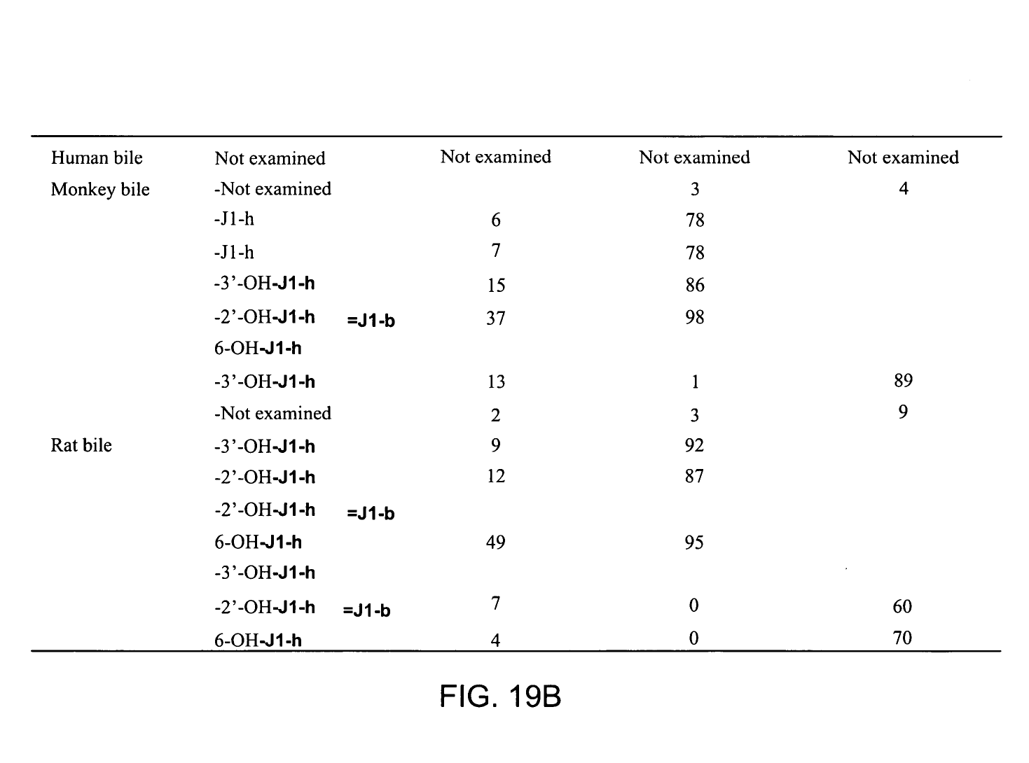

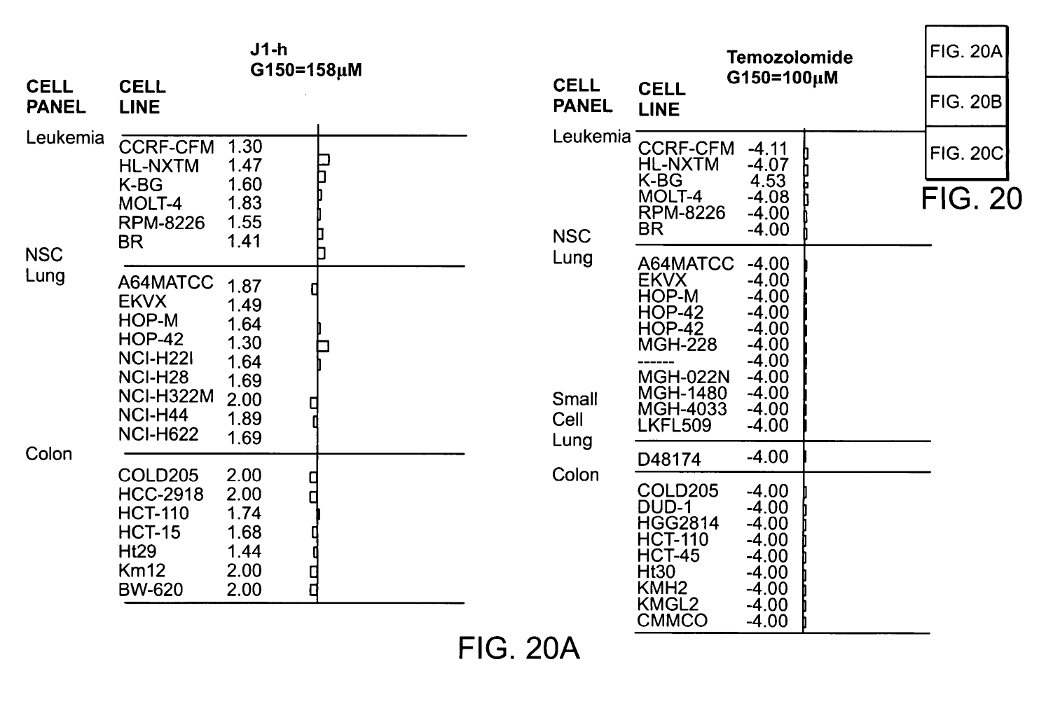

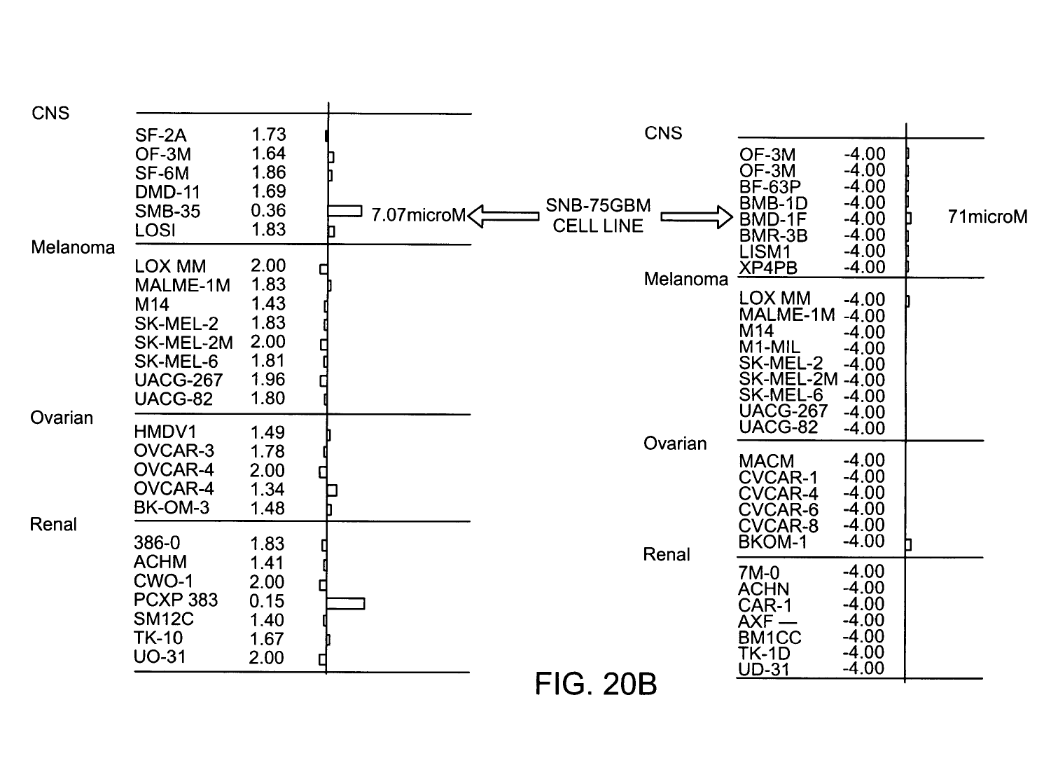

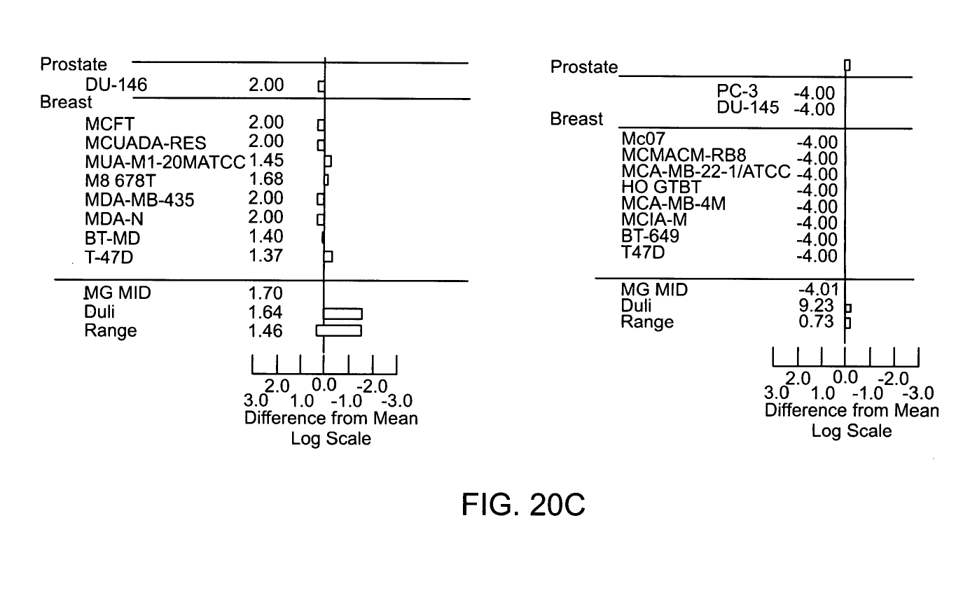

The chemical formulas of J1-b and J1-h, two analogues of J1 are shown in FIG. 18, which also shows glioma cytotoxic effects of these analogues. Interestingly, J1-b is one of the major metabolic products of J1-h in human body. FIG. 19 shows the presence of J1 metabolites in human, monkey, and rat urine, plasma, and bile. The cytotoxic effects of J1 analogues on U87, U118, and A172 glioma cells are shown in FIG. 21.

### Example 11

### J1 and J1-h Reduced the Viability of Prostate Carcinoma Cells and Human Hepatocellular Liver Carcinoma Cells

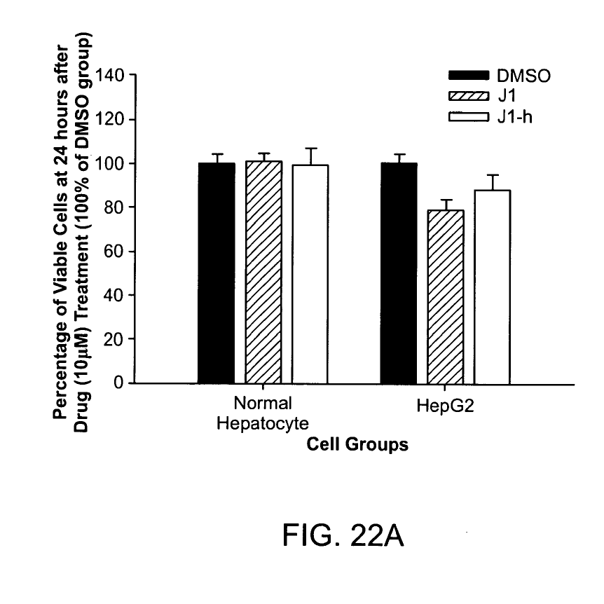

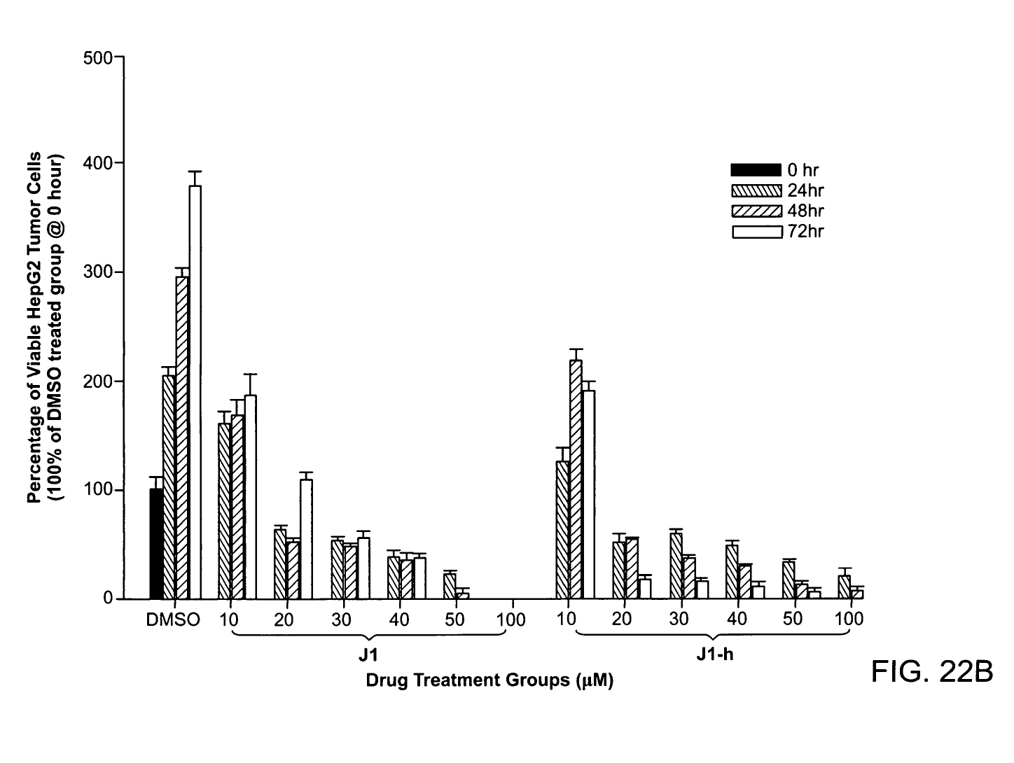

J1 and J1-h had little cytotoxic effect on primary cultured hepatocytes at 24 hours after drug treatment. Interestingly, both drugs had significant cytotoxic effects on the Human hepatocellular liver carcinoma cell line, HepG2, that were dose and time-dependent (FIGS. 22A and 22B).

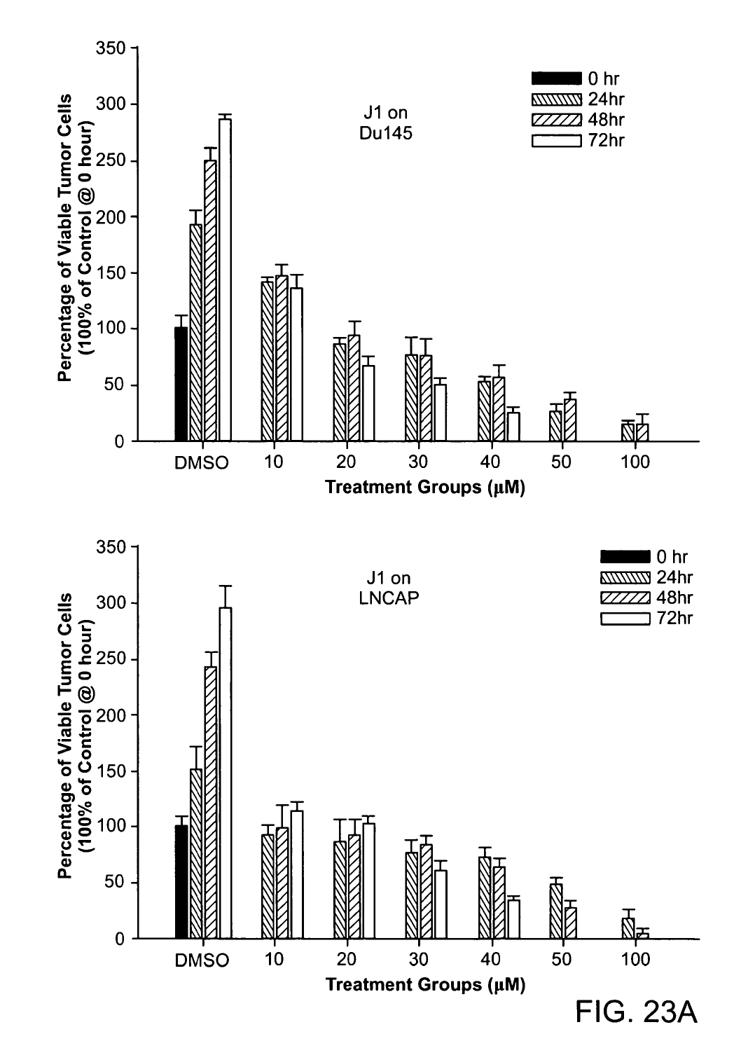

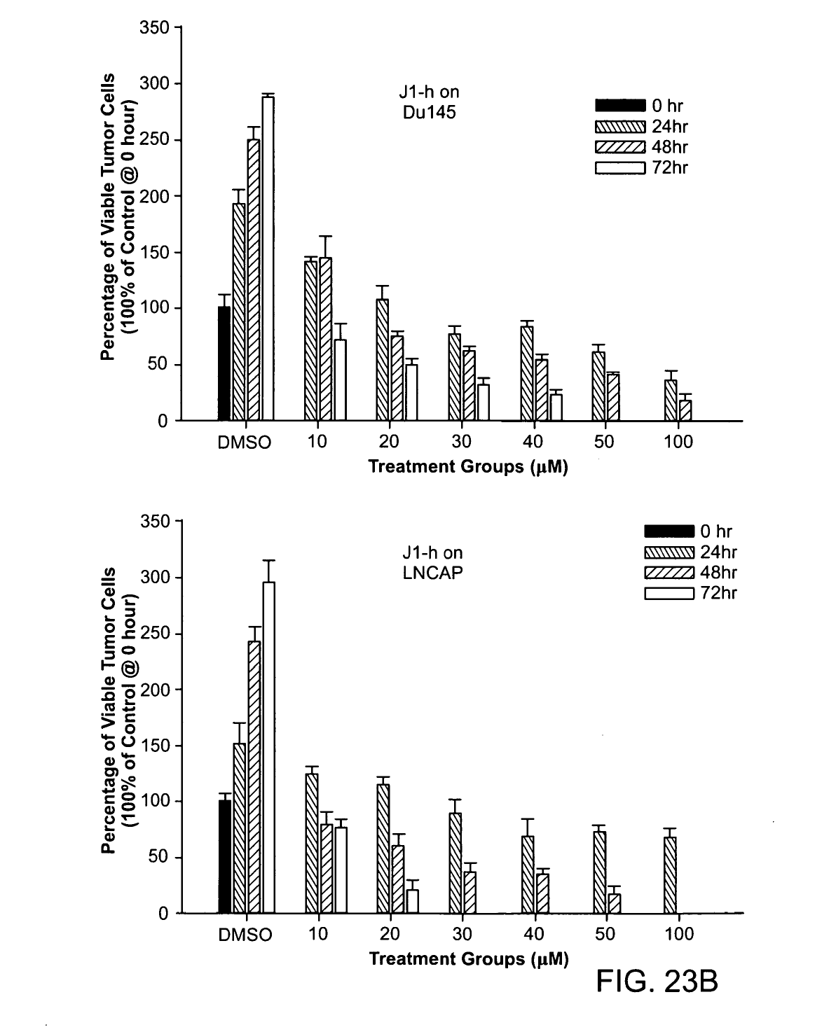

J1 and J1-h also exhibited strong dose and time-dependent cytotoxic effects on prostate carcinoma cells DU145 and LNCAP cells (FIG. 23).

### Example 12

### Dose- and Time-Dependent Cytotoxic Effects of J1, J1-b and Triclocarban Administration Were Observed to Vary Across Administered Concentrations

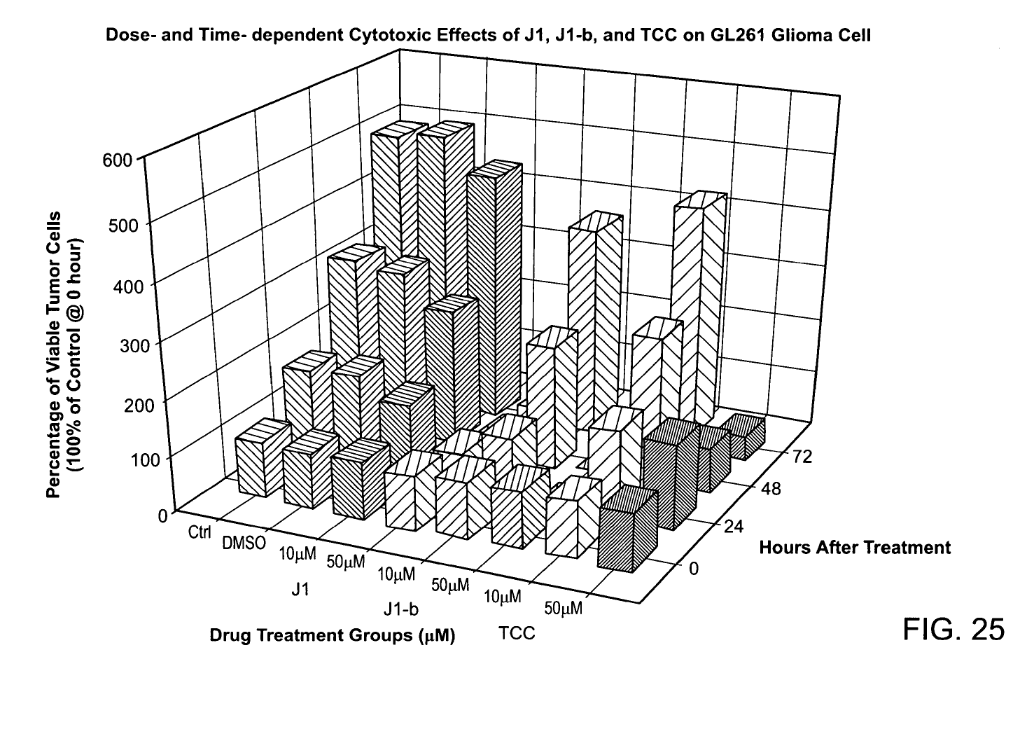

Cytotoxic effects of J1, J1-b and TCC agents were assessed in mouse GL261 glioma cells (FIG. 25). Increasing concentrations of all compounds showed progressively greater cytotoxic effects.

The results described above were obtained using the following methods and materials.

### Cell Culture and Transfection

T98G, U87MG, U118MG, A172, LN18, DBTRG-05MG, LN229, M059J, WT4 human glioma cell lines were purchased from American Type Culture Collection (Rockville, Md.). Cell culture was conducted according to the ATCC's recommendations, available at www.atcc.org. Normal human astrocytes (NHA) were purchased from Lonza Bioscience (Switzerland). Cell cultures were grown according to the vendor's recommendations for each cell line. GL261 mouse glioma cell line was obtained from NCI (Frederick, Md.) and was grown in RPMI (Gibco BRL, Grand Island, N.Y.) containing 10% FBS, 1% Penicillin-Streptomycin, and 4 mM L-glutamine at 37° C. with 5% CO₂.

### Tissue Specimens

Glioma tissue and non-neoplastic control tissue were obtained from Human Brain Tissue Repositary located at McKnight Brain Institute with IRB approval (252-2006).

### Tissue Selection, Donor Block Preparation, and Tissue Microarray (TMA) Construction

The tissue microarray was constructed as described in publications (Kononen et al., Nat Med 1998; 4:844-7; Camp et al., Lab Invest 2000; 80:1943-9; Rimm et al., Cancer J 2001; 7:24-31; Qiu et al., Lab Invest 88, 908-909, 2008) and according to the manufacturer's instructions (Beecher Instruments, Sun Prairie, Wis.). The tissue microarray included samples from 40 primary brain tumors as well as non-neoplastic control brain tissues (both cortex and white matter) with a total of 90 tissue cores. The array contained 13 WHO grade IV (glioblastomas) (All 13 GBM cases in the glioma tissue microarray were primary GBM cases based on clinical information), 6 Grade III (anaplastic) astrocytomas, 12 anaplastic oligodendrogliomas (WHO Grade III), and 7 control brain tissues.

Briefly, two neuropathologists chose the brain tumor bank tissues based on their pathological diagnosis and obtained the corresponding hematoxylin and eosin (H&E) glass slides and paraffin-embedded blocks. All diagnoses were reviewed and tumors were classified according to the most recent 2007 WHO guidelines. Diagnoses were also correlated with the results of two color fluorescence in situ hybridization (FISH) studies to detect losses of chromosomes 1p and 19q (Qiu et al., Lab Invest 88, 908-909, 2008). The representative areas to be sampled were identified microscopically and circled on the H&E slide. The circled H&E glass slide was matched with the corresponding region on the block. Areas of interest (1.0 mm in diameter) were punched out from the donor block by using the Beecher Tissue Microarrayer and placed into the receipient paraffin block following the pre-designed TMA grid map. Upon completion of the array, the TMA block was placed into a 37° C. oven for 15 minutes face down on a clean glass slide followed by applying gentle and even pressure against the glass slide. The TMA was immediately placed onto a block of ice for cooling. Paraffin sections were cut at 4-5 μm and air dried overnight in a vertical position. Array-section slides were stored labeled, stacked, wrapped tightly with Parafilm, and stored at −80° C. for long term use.

### Drugs

J1 and its derivatives were provided by NCI developmental therapeutic program repository or purchased from Sigma-Aldrich Inc. Temozolomide (TMZ) was obtained from Schering-Plough (Kenilworth, N.J.). Both J1 and TMZ were dissolved in DMSO and stock solution of 100 mM were made.

### RNA, DNA Extraction, PCR

RNA, DNA extraction, and PCR were performed according to QIAGEN's instructions.

### Western Blot Analysis

Whole cell protein extracts were prepared using cold lysis buffer consisting of: M-Per mammalian protein extraction reagent (Pierce Biochemical, Rockford, Ill.), 30 mM sodium fluoride, 1 mM sodium vanadate, and protease inhibitor cocktail tablets (Boehringer Mannheim). Samples were incubated on ice for 10 minutes and supernatants were recovered by centrifuging at 14,000 rpm at 4° C. for 20 minutes. Protein concentrations were determined by the BCA method (Pierce Biochemical). Proteins were separated on SDS-PAGE and transferred to nitrocellulose membrane (Osmonics, Minnetonka, Minn.). Blocking reagent is 5% nonfat dry milk in PBS (pH 7.4). Washing buffer is PBS (pH 7.4) with 0.1% Tween 20. Primary antibodies (p53, ATM, CHK1, CHK2) are commercially available. JAZ primary antibodies are described in Yang et al., Blood 108:4136-4145, 2006. The respective HRP-conjugated secondary antibodies were purchased from Boehringer Mannheim. Signals are visualized by using ECL chemiluminescence (Amersham, Indianapolis, Ind.) or Supersignal chemiluminescence (Pierce Biochemical) and Kodak X-OMAT films. Data acquired as integrated densitometric values are transformed to percentages of the values obtained from control samples visualized on the same blot.

### Immunohistochemistry (IHC)

Immunohistochemistry analysis was performed as previously described (Yachnis et al., Journal of Neuropathology and Experimental Neurology 1997; 56:186-98). The rabbit anti-human JAZ primary antibody was generated as described previously (Yang et al., Blood 108: 4136-4145, 2006). The mouse anti-human p53 monoclonal antibody (DO-1) was purchased from Santa Cruz Biotechnology. The mouse anti-human Ki-67 monoclonal antibody (MIB-1) was purchased from DAKO. TMA slides were incubated with the antibodies at 4° C. overnight followed by incubation with biotinylated secondary antibodies (DakoCytomation, Denmark). The bound antibodies were visualized with avidin—biotinylated peroxidase complex and diaminobenzidine tetrachloride (DAB) on anti-JAZ, anti-p53, or anti-Ki67 antibody for single staining, or with DAB on anti-JAZ and Vector red on anti-p53 antibody for double immunostaining. Immunohistochemistry scoring was first conducted independently by two neuropathologists then pooled together. Quantification of IHC on TMA slides was performed using Image J software from NIH (Bethesda, Md.).

### Immunocytochemistry

Immunocytochemical analysis was performed on cytospin cell culture slides. The JAZ primary antibodies were provided by Dr. W. Stratford May's lab and are described in Yang et al., Blood 108:4136-4145, 2006. The cytospin slides were incubated with the antibodies overnight followed by incubation with biotinylated secondary antibodies (DakoCytomation, Copenhagen, Denmark). The bound antibodies were visualized with avidin—biotinylated peroxidase complex and diaminobenzidine tetrachloride.

### Immunofluorescence (IF)

Paraffin embedded tissue sections were first de-paraffinized and re-hydrated followed by trilogy antigen retrieval (Cell Marque, Austin, Tex.). For cell culture, glioma cells were plated on glass cover slips at 10⁴/well in 6 well plates. Tumor cell cultures were fixed in 4% paraformaldehyde in PBS for 10 minutes followed by PBS washing once for 10 minutes. Both tissue sections and fixed culture cells were blocked by incubating with horse serum for 1 hour at RT. After removing the serum, primary antibodies (rabbit anti-human JAZ antibody (1:10) (Yang et al., Blood 108: 4136-4145, 2006) and/or the mouse anti-human p53 monoclonal antibody (DO-1, Santa Cruz Biotechnology) were added for incubation at 4° C. overnight. After washing with PBS for three times, cells/sections were incubated with Donkey anti-rabbit (AlexaFluor 488; Molecular Probes; 1:500) and Goat anti-mouse (AlexaFluor 594; Molecular Probes; 1:500) secondary antibodies in a dark environment for 2 hours at room temperature. Tissue sections were washed with PBS for 3 minutes×3 times and mounted on to slides with DAPI Vector Shield mounting medium (Vector Labs). For tumor cells, glass cover slips were removed from the 6 well plates and mounted on to slides with DAPI Vector Shield mounting medium (Vector Labs). For JAZ siRNA transfection experiments, tumor cells were fixed at 48 hours after the transfection.

### Organotypic Brain Slice Cultures

Organotypic brain slice cultures were performed as previously described with modification (Longo et al., Reprod Fertil Dev 1995; 7:385-9; Kohling et al., Brain Res 1996; 741:174-9; Toner et al., Brain Res 2002; 958:390-8). The non-neoplastic control brain tissues were obtained from Human Brain Tumor Tissue Repository located at University of Florida Mcknight Brain Institute under IRB's guideline. The brain tissues were sectioned coronally at 400 μm thickness with a vibratome (Leica S1000, Germany). The slices were separated and transferred to sterile, porous membrane units (0.4 μm; Millicell-CM, Millipore). The units were placed into 6-well trays containing 1 mL of culture medium in each dish (50% MEM with Earle's salts and 1-glutamine, 25% HBSS, 25% horse serum with 6.5 mg/L d-glucose, 20 mM HEPES and 50 U/mL streptomycin—penicillin, pH 7.2). Cultures were kept at 36-37° C. in 5% CO₂ for 24, 48, and 72 hours followed by sectioning at 10 μm thickness for H&E or immunohistochemistry.

siRNA Transfection

JAZ siRNA and control siRNA were purchased from Invitrogen. Cell transfection was performed by using METAFECTENE PRO (Biontex Laboratories GmbH, Germany) following the manufacturer's instructions. Briefly, 40 pmoles of JAZ (or control) siRNA was added into 30 μl serum-free medium then mixed with METAFECTENE PRO solution and incubated for 15-20 minutes at room temperature. The final siRNA-lipid complex solution was added into one 24-well-plate well. The complex-containing medium was replaced with serum-containing medium at 3 hours after the transfection to reduce the toxicity.

### Cell Viability Assay

Cell viability was measured by first seeding tumor cells in 24-well dishes at a density of 10,000 cells per well. Two days later, cells were transduced with JAZ (or control) siRNA. Forty-eight hours after transduction, cells were collected by trypsinization followed by the trypan blue exclusion method (Yang et al., J Biol Chem 1999; 274:27399-406) and counted manually using a hemacytometer. This experiment was repeated six times for each cell line and each time in triplicate.

### PCR and DNA Sequencing Analysis

Genomic DNA extraction, PCR, and DNA sequencing analysis were performed as described previously (31) with modifications. JAZ gene exons 1 to 7 were individually amplified from genomic DNA using PCR primers “Exon1 Forward (F): GCAAAGCAAAATAAGCGCGG, Reverse (R): ATGGGGAGCACAAGTCTGGG; Exon 2 (F): AGGCAGGGTTCAAAGGCAGT, (R): GGTTTGGCACCAGGTCTGAA; Exon 3 (F): CCTCACCTCCTCCTCAAAAC, (R): AGTCTGACTTGTTCACCACACC; Exon 4 (F): CAGTGGGGAACCATTGAAGA, (R): TGTGGCCTCAGTGAGGATCT; Exon 5 (F): GTTTGAACTCTCTCACTGCAGC, (R): CCCTACTTACAAGACCTAAGGAGG; Exon 6 (F): CGTCCTTAGAAGGGCATTTG, (R): GTTCTGATGCAGAAGTCCTGAG; Exon 7 (F): AAGGGCAGTGCTATCATTGC, (R): CCGTGTACTTTCCTGGTGAGA”. The amplified products were sequenced by the UF Interdisciplinary Center for Biotechnology Research (ICBR), using a custom sequencing primer that was complementary to an internal region of the PCR amplicon. Sequencing results from tumor samples and glioma cell lines were compared with that from normal brain tissue.

### Bisulfate Genomic Sequencing (BGS) and Methylation Specific PCR (MSP)

BGS and MSP were performed as described previously (Kim et al., Cancer Res 2006; 66:7490-501). PCR primer sequences used for BGS were “forward: GAGGTAATTGGATGTATATAGG; reverse: ATGAATTGGGTTGGTTGTTTAG”. TaqGold (ABI) thermostable DNA polymerase was used for all reactions. The band was purified from the agarose gel using the Qiaex II gel extraction kit (Qiagen) and cloned using the TA Cloning Kit as directed by the manufacturer (Invitrogen). Products from at least two independent PCR reactions were cloned and sequenced in a 96-well plate format using the M13 reverse and/or forward primers. All sequencing was performed at the UF ICBR. For MSP, reaction optimization was done to ensure that the PCR was in the linear amplification range. PCR primer sequences used for MSP were methylated forward TATTTATTTAAATTTCGCGATATTTAGGC; methylated reverse: GAACTACTATAAAACCCGACCG; unmethylated forward: TATTTATTAAATTTTGTGATATTTAGGT; unmethylated reverse: CAAACTACTATAAAACCCAACCA”. Specificity of MSP primers was routinely validated using human sperm genomic DNA unmethylated or in vitro methylated with CpG Methylase (New England Biolabs, Ipswich, Mass.). PCR products were resolved on 2% agarose gels.

### Statistical Analysis:

Student's T-test or One-way ANOVA with Dunnett's multiple comparison tests were performed on the data using SPSS statistical package (Chicago, Ill.). All values are given as mean±SD. Differences are considered significant if p<0.05.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A pharmaceutical composition for the treatment of a neoplasia comprising an effective amount of a substituted diphenyl urea compound and a pharmaceutically acceptable excipient.

2. The pharmaceutical composition of claim 1, wherein the diphenyl urea compound is represented by the formula: 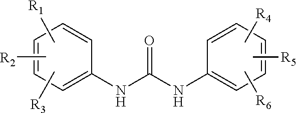
in which
each R₁, R₂, R₃, R₄, R₅, and R₆ is independently selected from the group consisting of H, Cl, and OH;
or a pharmaceutically acceptable salt or solvate thereof.

3. The pharmaceutical composition of claim 2, wherein at least one of R₁, R₂, or R₃ is Cl or OH.

4. The pharmaceutical composition of claim 3, wherein at least one of R₄, R₅, or R₆ is Cl or OH.

5. The pharmaceutical composition of claim 2, wherein the diphenyl urea compound is represented by the formula: 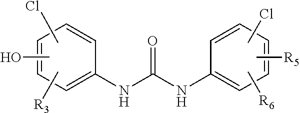
in which R₃, R₄, R₅, and R₆ are each independently selected from the group consisting of H, Cl, and OH.

6. The pharmaceutical composition of claim 5, wherein the diphenyl urea compound is represented by the formula:

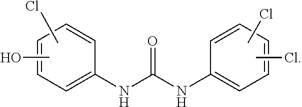

7. The pharmaceutical composition of claim 2, wherein the diphenyl urea compound is represented by the formula: 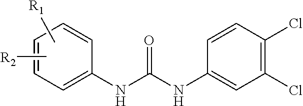
in which
R₁ and R₂ are each independently selected from the group consisting of H, Cl, and OH;
or a pharmaceutically acceptable salt or solvate thereof.

8. The pharmaceutical composition of claim 7, wherein R₁ is H or Cl and R₂ is OH.

9. The pharmaceutical composition of claim 7, wherein the diphenyl urea compound is represented by the formula: 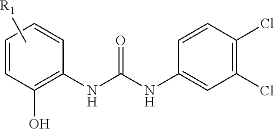
in which R₁ is H or Cl.

10. The pharmaceutical composition of any one of claims 1-9, wherein the compound is selected from the group consisting of J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea.

11. A method of ameliorating a neoplasia in a subject, the method comprising administering to the subject an effective amount of a compound of any one of claims 1-10.

12. A method of ameliorating a neoplasia in a subject, the method comprising administering to the subject an effective amount of an agent that reduces the expression or biological activity of a JAZ polypeptide in a cell, tissue, or organ relative to the level in an untreated control cell tissue or organ.

13. The method of claim 12, wherein the agent specifically binds a JAZ polypeptide.

14. The method of claim 11 or 12, the method further comprising administering temozolomide to the subject.

15. The method of claim 12, wherein the agent is a JAZ inhibitory nucleic acid molecule selected from the group consisting of an antisense, siRNA or shRNA molecule.

16. The method of claim 13, wherein the agent is a JAZ-specific antibody.

17. A method of ameliorating a neoplasia in a subject, the method comprising administering to the subject an effective amount of an agent that binds to a JAZ structural binding pocket sequence of a JAZ polypeptide.

18. The method of claim 17, wherein the agent is a compound according to any one of claims 1-10.

19. The method of claim 17, wherein the agent is a JAZ-specific antibody.

20. A method of reducing the survival of a neoplastic cell, the method comprising contacting the neoplastic cell with a compound of any one of claims 1-10.

21. The method of any one of claims 12-20, further comprising administering to the subject an effective amount of temozolomide.

22. The method of any one of claims 12-20, wherein the subject is a human.

23. The method of any one of claims 12-20, wherein the compound reduces neoplastic cell proliferation or invasiveness relative to the level in a corresponding neoplastic cell.

24. The method of any one of claims 12-20, wherein the neoplasia is selected from the group consisting of glioma, glioblastoma, hepatocellular carcinoma, lung cancer, leukemia, colon cancer, and prostate carcinoma.

25. A method for identifying an agent that binds to a JAZ polypeptide or fragment thereof, the method comprising
i) performing a computational fitting operation between the agent and a three-dimensional representation of a binding site of the JAZ polypeptide; and
ii) quantifying an association between the chemical entity and the three-dimensional representation of the binding pocket, thereby identifying an agent that reduces the biological activity of the JAZ polypeptide.

26. The method of claim 25, wherein a JAZ structural binding pocket sequence comprises an amino acid selected from the group consisting of GLN 17, HIS14, GLU10, GLU13, ARG9, GLU38, LYS41, ARG25, and ILE16.

27. The method of claim 25, wherein the JAZ structural binding pocket sequence comprises or consists essentially of:
vsg aqpvgreeve hmiqknqclf tntqckvcca llisesqk.

28. A method for identifying an agent that reduces JAZ biological activity, the method comprising
a) generating a three-dimensional representation of a JAZ structural binding pocket using the atomic coordinates of JAZ amino acid residues in the sequence; and
b) employing the three-dimensional structure to design or select a JAZ antagonist, thereby identifying an agent that reduces JAZ biological activity.

29. The method of claim 28, wherein the agent binds a JAZ structural binding pocket sequence.

30. The method of claim 25 or 28, wherein the agent is a substituted diphenyl urea.

31. The method of claim 30, wherein the agent is a compound selected from the group consisting of J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea.

32. The method of claim 25 or 28, the method further comprising contacting the agent with a JAZ expressing cell and detecting a reduction in JAZ expression or biological activity relative to a control cell.

33. The method of claim 32, wherein the cell is a cell in vitro or in vivo.

34. A method for identifying an agent for the treatment of a neoplasia, the method comprising
(a) contacting a JAZ expressing cell with a candidate agent; and
(b) detecting a reduction in the level of biological activity of a JAZ polypeptide in the contacted cell relative to a control cell, thereby identifying the agent as treating a neoplasia.

35. The method of claim 34, wherein the JAZ biological activity is detected by assaying proliferation, survival or invasiveness of a neoplastic cell.

36. The method of claim 34, wherein the agent specifically binds the JAZ polypeptide.

37. The method of claim 34, wherein the agent specifically binds the JAZ structural binding pocket sequence.

38. The method of claim 34, wherein the agent is an antibody or a substituted diphenyl urea compound.

39. A method for identifying a compound for the treatment of a neoplasia, the method comprising
(a) contacting a JAZ expressing cell with a test compound; and
(b) detecting a reduction in JAZ expression in the cell relative to an untreated control cell, thereby identifying the agent as treating a neoplasia.

40. The method of claim 39, wherein the reduction is in JAZ transcription or translation.

41. The method of claim 39, wherein the compound is a JAZ inhibitory nucleic acid molecule.

42. The method of claim 39, wherein the compound is an antisense, siRNA, or shRNA molecule.

43. A kit for the treatment of a neoplasia, the kit comprising an effective amount of a compound according to claims 1-10 and directions for using the kit for the treatment of a neoplasia.

44. The kit of claim 43, further comprising temozolomide.

45. A method of ameliorating a neoplasia in a subject, the method comprising administering to the subject an effective amount of the pharmaceutical composition of any one of claims 1-9 and an effective amount of an agent that reduces the expression or biological activity of a polypeptide selected from the group consisting of PBEF1, HSPA5, ANXA1, TPI1, ANXA5, HSPA9, TGM2, NPM1, RPLP2, HSPA1B, VIM, LMNA and HIST2H3PS2.

46. The method of claim 45, wherein the agent specifically binds said polypeptide.

47. The method of claim 45, wherein the method further comprising administering temozolomide to the subject.

48. The method of claim 45, wherein the agent is an inhibitory nucleic acid molecule selected from the group consisting of an antisense, siRNA or shRNA molecule.

49. The method of claim 45, wherein the agent is an antibody.

50. The method of claim 45, wherein said polypeptide is PLOD2. The method of claim 45, wherein said polypeptide is HSPA5.

51. The method of claim 45, wherein said polypeptide is PBEF1.

52. The method of claim 45, wherein said polypeptide is HIST2H3PS2.

53. A method of ameliorating a neoplasia in a subject, the method comprising administering to the subject an effective amount of an agent that increases the expression or biological activity of an ATF-4 or CHOP polypeptide.

54. The method of claim 53, wherein the subject is a human.

55. The method of claim 53, wherein the agent is a mammalian expression vector encoding human ATF-4.

56. The method of claim 53, wherein the agent is a mammalian expression vector encoding human CHOP.

57. The method of any one of claims 25-42, further comprising the step of contacting a cell with the agent and detecting a decrease in the expression PLOD2 relative to a control cell, thereby identifying an agent as useful for the treatment of neoplasia.

58. A method for identifying an agent that reduces JAZ biological activity, the method comprising
a) generating a three-dimensional representation of a JAZ structural binding pocket using the atomic coordinates of JAZ amino acid residues in the JAZ structural binding pocket sequence;
b) employing the three-dimensional structure to design or select a JAZ antagonist; and
iii) detecting a decrease in the expression PLOD2, thereby identifying an agent that reduces the biological activity of the JAZ polypeptide thereby identifying an agent that reduces JAZ biological activity.

59. A method for identifying an agent for the treatment of a neoplasia, the method comprising
(a) contacting a JAZ expressing cell with a candidate agent;
(b) detecting a reduction in the level of biological activity of a JAZ polypeptide in the contacted cell relative to a control cell; and
(c) detecting a decrease in the expression PLOD2, thereby identifying an agent that reduces the biological activity of the JAZ polypeptide thereby identifying an agent for the treatment of a neoplasia.

60. A method for identifying a compound for the treatment of a neoplasia, the method comprising
(a) contacting a JAZ expressing cell with a candidate agent;
(b) detecting a reduction in JAZ expression in the cell relative to an untreated control cell, and
(c) detecting a decrease in the expression PLOD2, thereby identifying the agent as treating a neoplasia.

61. A pharmaceutical composition comprising the pharmaceutical composition of any one of claims 1-9, and an effective amount of an inhibitory nucleic acid molecule selected from the group consisting of an antisense, siRNA or shRNA molecule, wherein said inhibitory nucleic acid molecule is sufficiently complementary to the transcript of a gene selected from the group consisting of PBEF1, HSPA5, ANXA1, TPI1, ANXA5, HSPA9, TGM2, NPM1, RPLP2, HSPA1B, VIM, LMNA and HIST2H3PS2 to reduce the expression of said gene in a mammalian cell, and a pharmaceutically-acceptable carrier.

62. The pharmaceutical composition of claim 60, wherein said gene is HSPA5.

63. The pharmaceutical composition of claim 60, wherein said gene is PBEF1.

64. The pharmaceutical composition of claim 58, wherein said gene is HIST2H3PS2.

65. A kit for the treatment of a neoplasia, the kit comprising an effective amount of a compound according to any one of claims 1-9 and an agent identified according to the methods of any one of claims 58-60 and directions for using the kit for the treatment of a neoplasia.

66. A computer for producing a three-dimensional representation of
a) a molecule or molecular complex, wherein said molecule or molecular complex comprises a binding site defined by structure coordinates of amino acid residues of the JAZ protein; or
b) a three-dimensional representation of a homologue of said molecule or molecular complex, wherein said homologue comprises a binding pocket that has a root mean square deviation from the backbone atoms of said amino acids of not more than about 2.0 angstroms, wherein said computer comprises:
(i) a machine-readable data storage medium comprising a data storage material encoded with machine-readable data, wherein said data comprises the structure coordinates of structure coordinates of amino acid residues of the JAZ protein;
(ii) a working memory for storing instructions for processing said machine-readable data;
(iii) a central-processing unit coupled to said working memory and to said machine-readable data storage medium for processing said machine readable data into said three-dimensional representation; and
(iv) a display coupled to said central-processing unit for displaying said three-dimensional representation.

67. The computer of claim 66, wherein the structure coordinates comprise those shown in FIG. 27.

68. A method for evaluating the potential of a chemical entity to associate with a) a molecule or molecular complex comprising a binding pocket defined by structure coordinates of amino acid residues of the JAZ protein, or b) a homologue of said molecule or molecular complex, wherein said homologue comprises a binding pocket that has a root mean square deviation from the backbone atoms of said amino acids of not more than 2.0 angstroms,
the method comprising the steps of:
i) employing computational means to perform a fitting operation between the chemical entity and a binding pocket of the molecule or molecular complex; and
ii) analyzing the results of the fitting operation to quantify the association between the chemical entity and the binding pocket.

69. A method for evaluating the potential of a chemical entity to bind with
a) a molecule or molecular complex comprising a binding pocket defined by structure coordinates of one or more of amino acid residues of the JAZ protein, or b) a homologue of said molecule or molecular complex, wherein said homologue comprises a binding pocket that has a root mean square deviation from the backbone atoms of said amino acids of not more than 2.0 angstroms,
the method comprising the steps of:
i) employing computational means to perform a fitting operation between the chemical entity and a binding pocket of the molecule or molecular complex; and
ii) analyzing the results of the fitting operation to quantify the association between the chemical entity and the binding pocket.

70. The method of any of claims 66-69, wherein the JAZ structural binding pocket comprises at least a fragment of the following amino acid sequence:
vsg aqpvgreeve hmiqknqclf tntqckvcca llisesqk.

71. The method of any of claims 66-69, wherein the JAZ structural binding pocket comprises one or more of GLN 17, HIS14, GLU10, GLU13, Arg9, GLU38, LYS41, ARG25, and ILe161.

72. A method for ameliorating a neoplasia in a subject, the method comprising administering to the subject an effective amount of a compound capable of disrupting binding of a JAZ protein with a nucleic acid molecule, such that a neoplasia is ameliorated in the subject.

73. The method of claim 70, wherein the compound is selected from the group consisting of J1, N′-(2-hydroxy-phenyl)-N-(3,4-dichlorophenyl)Urea, J1-b, 1-(4-chloro-2-hydroxy-phenyl)-N-(3,4-dichlorophenyl) Urea, J1-h, 3-(4-chloro-phenyl)-1-(3,4-dichlorophenyl) Urea, N-(3-chlorophenyl)-N′-(2-hydroxyphenyl) Urea, N-(3,4-dichlorophenyl)-N′-(3-hydroxyphenyl) Urea, N-(3,4-dichlorophenyl)-N′-(4-hydroxyphenyl) Urea, 1-(5-chloro-2-hydroxyphenyl)-3-(3,4-dichlorophenyl)urea, 3,4-Dichlorophenylurea, 1-(3′,4′-dichlorophenyl)-3-(4′-chlorophenyl) Urea, 3-(4-chloro-2-hydroxy-phenyl)-1-(4,5-dichloro-2-hydroxy-phenyl)urea, 3-(4-chlorophenyl)-1-(4,5-dichloro-2-hydroxy-phenyl)urea, 1-(5-chloro-2-hydroxy-phenyl)-3-(4-chlorophenyl)urea, 3-(4-chlorophenyl)-1-(2-hydroxyphenyl)urea, 1-(5-chloro-2-hydroxy-phenyl)-3-phenyl-urea, 1-(5-chloro-2-hydroxy-phenyl)-3-(3,5-dichlorophenyl)urea, and 1-(3,4-dichloro-2-hydroxy-phenyl)-3-(2,3-dichlorophenyl)urea, or a pharmaceutically acceptable salt thereof.
